(19) United States
(12) Patent Application Publication
(10) Pub. No.: US 2004/0191843 A1
(43) Pub. Date: Sep. 30, 2004

(54) Cell-killing molecules and methods of use thereof

(75) Inventors: Susan C. Wright, Saratoga, CA (US); James W. Larrick, Woodside, CA (US); David S. Wilson, Mountain View, CA (US); Steffen R. Nock, Redwood City, CA (US)

Correspondence Address: MEDLEN & CARROLL, LLP, Suite 350, 101 Howard Street, San Francisco, CA, 94105, US

(73) Assignee: Palo Alto Institute of Molecular Medicine

(21) Appl. No.: 10/770,668

(22) Filed: Feb. 2, 2004

Related U.S. Application Data
(60) Provisional application No. 60/444,191, filed on Feb. 3, 2003.
(60) Provisional application No. 60/460,855, filed on Apr. 8, 2003.

(51) Int. Cl. G01N 33/574; A61K 39/395

(52) U.S. Cl. 435/7.23; 424/178.1

(57) ABSTRACT

The invention provides compositions comprising amino acid sequences that have cell killing activity, nucleic acid sequences encoding them, antibodies that specifically bind with them, and methods of using these compositions for increasing and/or reducing cell death, detecting cell death, diagnosing diseases associated with altered cell death, and methods for identifying test agents that alter cell death.

This application claims priority to U.S. Provisional Application Serial No. 60/444,191, filed Feb. 3, 2003 and 60/460,855, filed Apr. 8, 2003, the contents of each of which are incorporated herein in their entirety.

This invention was made, in part, with government support under grant number CA68223 awarded by the National Institutes of Health. The U.S. government has certain rights in the invention.

### FIELD OF THE INVENTION

This invention relates to the fields of cell death, including apoptosis and necrosis. In particular, the invention relates to compositions comprising amino acid sequences that have cell killing activity, nucleic acid sequences encoding them, antibodies that specifically bind with them, and methods of using these compositions for increasing and/or reducing cell death, detecting cell death, diagnosing diseases associated with altered cell death, and methods for identifying test agents that alter cell death. The invention also relates to the fields of tumor biology, medical diagnostics, and proteomics.

### BACKGROUND OF THE INVENTION

Several diseases are associated with undesirable cell proliferation, such as cancer, tumor metastasis, angiogenesis, restenosis, atherosclerosis, fibrosis, hemangioma, lymphoma, leukemia, psoriasis, arthritis, autoimmune disease, diabetes, amyotrophic lateral sclerosis, graft rejection, retinopathy, macular degeneration, and retinal tearing. Some of these diseases, such as cancer, are associated with a high mortality rate.

Current treatments for diseases associated with undesirable cell proliferation rely mainly on treatments which are not selective for the disease but which have deleterious effects on other organs of the body. For example, chemotherapeutic reagents or radiation have serious side effects because they kill or impair all proliferating cells in the body, including healthy cells. Side effects are unpleasant and often create health problems that themselves increase patient mortality.

Another approach to treating these diseases, such as cancer, employs the use of immunotoxins. However, the development of immunotoxins that are selectively cytotoxic to cancer cells and that remain harmless to non-cancerous cells of the patient has been stymied by the development of immune responses in patients to foreign proteins which comprise the immunotoxins. Immune responses against murine monoclonal antibodies and anti-toxin antibodies have been detected in both animals and humans treated with immunotoxins. While advances in humanization techniques have alleviated some of the immunogenicity associated with the antibody portion of immunotoxins, humanization of the targeting portion of the toxin does not counter the immunogenicity of the toxic moiety.

In order to overcome the immunogenicity of the toxins, secreted human ribonucleases have been used as the toxic portions of immunotoxins. However the use of secreted nucleases to induce cell death has several major drawbacks. First, these often disulfide-bridge containing nucleases are deactivated in the highly reducing intracellular environment because these nucleases have not evolved to tolerate such an environment, but rather have evolved in the oxidizing environment of the extracellular space. Another drawback of nucleases lies in the inability to produce them recombinantly at high levels in transformed cells because intracellular expression of free nucleases or nuclease antibody conjugates may kill the cell, while secreted expression may be at low levels.

Thus, there remains a need for compositions and methods for diagnosing and reducing symptoms of diseases that are associated with undesirable cell proliferation. Preferably, the compositions may be modified to confer to them specificity with respect to target cells of interest. Preferably, also, the cytotoxicity of these compositions is not inactivated by the immune system, the compositions are readily manufactured, and may be modified to increase their specificity for target cells of interest.

### SUMMARY OF THE INVENTION

The invention provides a composition comprising an isolated amino acid sequence that comprises a portion of SEQ ID NO:4, wherein the portion comprises SEQ ID NO:6 and has activity chosen from DNA nuclease activity and cell killing activity, and more preferably, wherein the portion comprises SEQ ID NO:7.

Also provided herein is a composition comprising a conjugate that comprises a mitochondrial protein, wherein the mitochondrial protein has activity chosen from DNA nuclease activity and cell killing activity, and is operably linked to a first molecule that specifically binds to a cell molecule. In one embodiment, the mitochondrial protein is chosen from SEQ ID NO:6 and SEQ ID NO:7. In another embodiment, the mitochondrial protein comprises an amino acid sequence chosen from SEQ ID NO:8, SEQ ID NO:10, SEQ ID NO:12, SEQ ID NO:24, SEQ ID NO:72, SEQ ID NO:74, SEQ ID NO:76, and SEQ ID NO:78.

Also provided is a composition comprising a conjugate that comprises a protein chosen from SEQ ID NO:14, SEQ ID NO:18, SEQ ID NO:20, SEQ ID NO:22, SEQ ID NO:26, SEQ ID NO:40, and SEQ ID NO:42, wherein the protein has activity chosen from DNA nuclease activity and cell killing activity, and is operably linked to a first molecule that specifically binds to a cell molecule.

Optionally, the conjugates of the invention further contain one or more of a N-terminal signal peptide, cell internalization peptide, and a nuclear localization peptide. While not intending to limit the nature or source of the first molecule, in one embodiment, the molecule comprises an antibody, preferably an antibody that specifically binds to cancer cells, such as, without limitation, non-small cell lung carcinoma cells, breast cancer cells, gastrointestinal cancer cells, renal carcinoma cells, liver cancer cells, B cell lymphoma cells, myeloid leukemia cells, renal carcinoma cells, colon cancer cells, pancreatic cancer cells, colorectal cancer cells, ovarian cancer cells, and prostate cancer cells. In one embodiment, the cancer cells comprise liver cancer cells, such as hepatocellular cancer cells. In another embodiment, the antibody that binds to liver cancer cells comprises an antibody chosen from Hepama-1, anti-PLC 1, anti-PLC2, K-PLC1, K-PLC2, K-PLC3, 49-D6, 7-E10, 34-A4, 26-A10,34-B9,79-C8, 16-E10, 5D3, 5C3, 2C6, a-AFP, HP-1, hHP-1, mAb 95, YPC2/38.8, P215457, PM4E9917, HAb25, HAb27, KY-1, KY-2, KY-3, 9403 Mab, KM-2, S1, 9B2, IB1, A9-84, SF-25, AF-10, XF-8, AF-20, a-hIRS-1, FB-50, SF 31, SF 90,2A3D2, and 2D11E2. In a particularly preferred embodiment, the antibody that binds to liver cancer cells comprises Hepama-1 antibody, and is more preferably a humanized Hepama-1. In a further embodiment, the first molecule comprises a ligand of a cell receptor, preferably exemplified by a ligand that comprises a growth factor. In one embodiment, the growth factor is chosen from epidermal growth factor, insulin-like growth factor, fibroblast growth factor, and vascular endothelial growth factor.

Additionally provided herein is a composition comprising an isolated amino acid sequence that comprises a portion of SEQ ID NO:4, wherein the portion comprises SEQ ID NO:6 and has activity chosen from DNA nuclease activity and cell killing activity. In one embodiment, the portion comprises SEQ ID NO:7, and optionally further comprises one or more of N-terminal signal peptide, cell internalization peptide, nuclear localization peptide, and an antibody that specifically binds to biotin.

The invention also provides a composition comprising an expression vector that comprises a nucleic acid sequence encoding an amino acid sequence that comprises a portion of SEQ ID NO:4, wherein the portion comprises SEQ ID NO:6, and wherein the amino acid sequence has activity chosen from DNA nuclease activity and cell killing activity. Preferably, the portion comprises SEQ ID NO:7.

In addition, the invention provides a composition that comprises a cell comprising an expression vector that comprises a nucleic acid sequence encoding an amino acid sequence that comprises a portion of SEQ ID NO:4, wherein the portion comprises SEQ ID NO:6, and wherein the amino acid sequence has activity chosen from DNA nuclease activity and cell killing activity, preferably wherein the portion comprises SEQ ID NO:7.

The invention also provides a composition comprising an antibody that specifically binds to SEQ ID NO:7. In one embodiment, the binding affinity of the antibody to one or more of SEQ ID NO:6 and SEQ ID NO:7 is higher than the binding affinity of the antibody to SEQ ID NO:4. Preferably, binding of the antibody reduces SEQ ID NO:7 activity chosen from DNA nuclease activity and cell killing activity.

Also provided by the invention is a method for increasing cell death, comprising: a) providing: i) cells; and ii) a composition comprising an amino acid sequence chosen from SEQ ID NO:6, SEQ ID NO:7, SEQ ID NO:8, SEQ ID NO:10, SEQ ID NO:12, SEQ ID NO:24, SEQ ID NO:72, SEQ ID NO:74, SEQ ID NO:76, SEQ ID NO:78, SEQ ID NO:14, SEQ ID NO:18, SEQ ID NO:20, SEQ ID NO:22, SEQ ID NO:26, SEQ ID NO:40, and SEQ ID NO:42; and b) contacting the cells with the composition to produce contacted cells wherein the contacting increases cell death of the contacted cells. In one embodiment, the amino acid sequence comprises SEQ ID NO:7. In one embodiment, the amino acid sequence is operably linked to an antibody that specifically binds to the cells. Preferably, the method further comprises detecting increased cell death in the contacted cells. In one embodiment, the method further comprises, prior to step b), providing a nucleotide sequence encoding the amino acid sequence, and expressing the nucleotide sequence in the cells. The cells used in the invention's methods may be in vitro or in vivo in a mammalian animal, such as a human. Preferably, the human is chosen from a human that has cancer and a human that is suspected of being capble of developing cancer. Preferably, the amino acid sequence is operably linked to an antibody that specifically binds to cancer cells in the cancer. Alternatively, the cancer is chosen from liver cancer, gastric cancer, head cancer, neck cancer, lung cancer, breast cancer, prostate cancer, cervical cancer, pancreatic cancer, colon cancer, ovarian cancer, stomach cancer, esophagus cancer, mouth cancer, tongue cancer, gum cancer, skin cancer, muscle cancer, heart cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, gall bladder cancer, ocular cancer, joint cancer, glioblastoma, mesothelioma, lymphoma, leukemia, melanoma, squamous cell carcinoma, osteosarcoma, and Kaposi's sarcoma. Preferably the cancer is liver cancer, and the antibody that specifically binds to liver cancer cells comprises Hepama-1 antibody.

The invention also provides a method for detecting cell apoptosis, comprising detecting a sequence chosen from SEQ ID NO:6 and SEQ ID NO:7 in the cytoplasm of the cell, and preferably further comprises quantifying the level of the detected sequence.

Also provided by the invention is a method for detecting disease in a mammalian animal, comprising detecting SEQ ID NO:6 and/or SEQ ID NO:7 in the blood of the mammalian animal, preferably wherein the disease is associated with cell death (such as increased and decreased cell death).

### BRIEF DESCRIPTION OF THE DRAWINGS

FIG. 1 shows the amino acid sequence (SEQ ID NO:1) of mature porcine mitochondrial MDH. The sequence obtained from the 18 kDa peptide is underlined with a dashed line, whereas the sequence obtained from the 9 kDa peptide is underlined with a solid line.

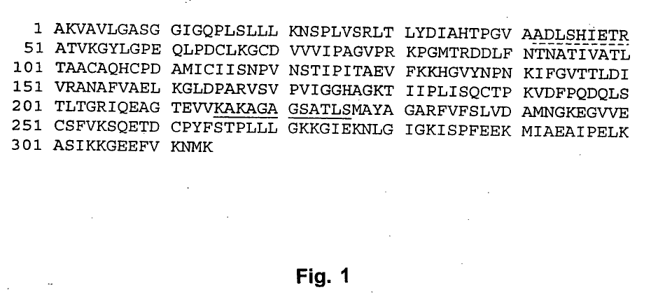

FIG. 2 shows that rADF induces DNA fragmentation in isolated normal nuclei. (A) Normal nuclei isolated from U937 or MCF-7 cells were incubated with the indicated concentration of rADF for 4 h, and DNA fragmentation measured by release of ³H-labeled DNA fragments. (B) U937 nuclei were incubated with rADF or control sample prepared from E. coli transfected with vector alone for 4 h and DNA analyzed by agarose gel electrophoresis.

FIG. 3 shows that rADF activates nucleases endogenous to normal nuclei. U937 nuclei were incubated with and without rADF for 20 h, then nuclear extracts prepared and tested at different dilutions for DNase activity on naked DNA substrate as described in detail previously (Wright, et al., 1994, 1996).

FIG. 4 shows UV light-induced translocation of ADF from Mitochondria (Mito) to Cytosol and Nucleus in HL-60 Cells is prevented by overexpression of Bcl-2. Cells were treated with and without UV light, incubated for 4 h, and subcellular fractions and rADF (positive control) were analyzed for ADF by western blotting.

FIG. 5 shows anti-rADF immuno-depletes 9 kD ADF and nuclear DNA fragmenting activity but not CAD from apoptotic cytosol. (A) Cytosols from frankly apoptotic UV light treated U937 cells or rADF 50 nM alone (as a positive control) were adsorbed with beads coated with control IgG, anti-rADF, or anti-caspase 3, and then tested for induction of DNA fragmentation in isolated nuclei in a 4 h assay measuring release of ³H-labeled fragments. Caspase 3 was adsorbed with the same reagents and tested for proteolytic activity on the synthetic substrate, DEVD-pNa. (B) Apoptotic cytosols adsorbed as in (A) were evaluated for the presence of ADF by western blot. (C) Control cytosol and nuclear extracts, extracts from nuclei of apoptotic cells, and apoptotic cytosols adsorbed as in A were evaluated for the presence of CAD by western blot.

FIG. 6 shows rADF-Ant fusion protein induces internucleosomal DNA fragmentation in intact U937 cells. U937 cells were treated with the indicated concentrations of rADF-Ant, rADF alone, free penetratin (Pen) alone, or rADF combined with free Pen for 4 h, and DNA fragmentation measured by release of ³H-labeled fragments (A) or by agarose gel electrophoresis (B).

FIG. 7 shows HL-60 cells overexpressing Bcl-2 are still sensitive to DNA fragmentation induced by rADF-Ant. HL-60 neo or HL-60 Bcl-2 were treated with the indicated concentration of inducing agents and DNA fragmentation was measured 4 h later by release of ³H-labeled DNA fragments.

FIG. 8 shows heat shock-treated U937 cells are still sensitive to rADF-Ant-induced DNA Fragmentation. U937 cells were heated at 42° C. for 30 min, then cultured to allow recovery for 3 h and tested along with control U937 cells for DNA fragmentation in response to the indicated inducing agent in a 4 h assay measuring release of ³H-labeled DNA fragments.

### DEFINITIONS

To facilitate understanding of the invention, a number of terms are defined below. Unless otherwise indicated, all technical and scientific terms used herein have the same meaning as in Sambrook et al. (2001) “Molecular Cloning: A Laboratory Manual” Cold Spring Harbor Press, 3rd Ed.; and Ausubel, F. M., et al. (1993) in Current Protocols in Molecular Biology.

The terms “peptide,” “peptide sequence,” “amino acid sequence,” “polypeptide,” “polypeptide sequence” and “protein” are used interchangeably herein to refer to a biopolymer composed of two or more amino acid or amino acid analog subunits, typically some or all of the 20 common L-amino acids found in biological proteins, linked by peptide linkages, or other linkages. The term peptide includes molecules which are commonly referred to as peptides, which generally contain from about two (2) to about twenty (20) amino acids. The term peptide also includes molecules which are commonly referred to as polypeptides, which generally contain from about twenty (20) to about fifty amino acids (50). The term peptide also includes molecules which are commonly referred to as proteins, which generally contain from about fifty (50) to about three thousand (3000) amino acids. A peptide, polypeptide or protein may be synthetic, recombinant or naturally occurring. A synthetic peptide is a peptide which is produced by artificial means in vitro (e.g., was not produced in vivo).

The terms “protease,” “proteolytic enzyme,” and “proteinase” refers to an enzyme that catalyzes hydrolysis of peptide bonds between amino acid residues.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “expression” refers to the process by which a polypeptide is produced based on the nucleic acid sequence of a gene. The process includes both transcription and translation.

The term “polymerase chain reaction” (“PCR”) refers to a reaction in which copies are made of a target polynucleotide using one or more primers, and a catalyst of polymerization, such as a reverse transcriptase or a DNA polymerase, and particularly a thermally stable polymerase enzyme. Methods for PCR are taught in U.S. Pat. No. 4,683,195 (Mullis) and U.S. Pat. No. 4,683,202 (Mullis et al.). All processes of producing replicate copies of the same polynucleotide, such as PCR or gene cloning, are collectively referred to herein as “replication.”

The term “intracellular protein” means a protein which resides inside the cytoplasm and/or nucleus of a cell. Generally, intracellular proteins are not secreted under normal physiological conditions, but may be found in secreted vesicles and/or in the extracellular space under certain conditions (such as cell lysis, cell death, etc.).

As used herein, any verb ending in ‘ing,’ such as “providing,” “contacting,” “mixing,” “spreading,” “positioning,” “observing,” “transmitting,” is intended to recite an act rather than a function and/or result.

As used herein, the singular forms “a,” “an” and “the” include both singular and plural references unless the content clearly dictates otherwise.

As used herein, the term “or” when used in the expression “A or B,” where A and B refer to a composition, disease, product, etc., means one, or the other, or both.

The terms “chosen from A, B and C” and “chosen from one or more of A, B and C” are equivalent terms that mean selecting any one of A, B, and C, or any combination of A, B, and C.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc.

Unless otherwise indicated, all numbers expressing quantities of ingredients, properties such as molecular weight, reaction conditions, and so forth as used herein, are to be understood as being modified in all instances by the term “about.” Accordingly, unless indicated to the contrary, the numerical parameters herein are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (e.g., amino acid sequence such as MDH, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, and HIV Tat protein, etc., nucleic acid sequence such as those encoding any of the polypeptides described herein), and/or phenomenon (e.g., cell death, cell apoptosis, cell viability, cell survival, binding to a molecule, expression of a nucleic acid sequence, transcription of a nucleic acid sequence, enzyme activity, etc.) means that only the particularly named molecule or phenomenon is excluded.

The term “altering” and grammatical equivalents as used herein in reference to the level of any molecule (e.g., amino acid sequence such as MDH, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, and HIV Tat protein, etc., and nucleic acid sequence such as those encoding any of the polypeptides described herein), and/or phenomenon (e.g., cell death, cell apoptosis, cell viability, cell survival, binding to a molecule, expression of a nucleic acid sequence, transcription of a nucleic acid sequence, enzyme activity, etc.) refers to an increase and/or decrease in the quantity of the molecule and/or phenomenon, regardless of whether the quantity is determined objectively and/or subjectively.

Unless defined otherwise in reference to the level of molecules and/or phenomena, the terms “increase,” “elevate,” “raise,” and grammatical equivalents (including “higher,” “greater,” etc.) when in reference to the level of any molecule (e.g., amino acid sequence such as MDH, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, and HIV Tat protein, etc., and nucleic acid sequence such as those encoding any of the polypeptides described herein), and/or phenomenon (e.g., cell death, cell apoptosis, cell viability, cell survival, binding to a molecule, binding affinity, expression of a nucleic acid sequence, transcription of a nucleic acid sequence, enzyme activity, etc.) in a first sample relative to a second sample, mean that the quantity of the molecule and/or phenomenon in the first sample is higher than in the second sample by any amount that is statistically significant using any art-accepted statistical method of analysis. In one embodiment, the increase may be determined subjectively, for example when a patient refers to their subjective perception of disease symptoms, such as pain, difficulty in breathing, clarity of vision, nausea, tiredness, etc. In another embodiment, the quantity of the molecule and/or phenomenon in the first sample is at least 10% greater than, at least 25% greater than, at least 50% greater than, at least 75% greater than, and/or at least 90% greater than the quantity of the same molecule and/or phenomenon in a second sample.

Unless defined otherwise in reference to the level of molecules and/or phenomena, the terms “reduce,” “inhibit,” “diminish,” “suppress,” “decrease,” and grammatical equivalents (including “lower,” “smaller,” etc.) when in reference to the level of any molecule (e.g., amino acid sequence such as MDH, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, and HIV Tat protein, etc., and nucleic acid sequence such as those encoding any of the polypeptides described herein), and/or phenomenon (e.g., cell death, cell apoptosis, cell viability, cell survival, binding to a molecule, affinity of binding, expression of a nucleic acid sequence, transcription of a nucleic acid sequence, enzyme activity, etc.) in a first sample relative to a second sample, mean that the quantity of molecule and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, difficulty in breathing, clarity of vision, nausea, tiredness, etc. In another embodiment, the quantity of molecule and/or phenomenon in the first sample is at least 10% lower than, at least 25% lower than, at least 50% lower than, at least 75% lower than, and/or at least 90% lower than the quantity of the same molecule and/or phenomenon in a second sample.

A “composition” comprising a particular polynucleotide sequence and/or comprising a particular protein sequence as used herein refers broadly to any composition containing the recited polynucleotide sequence (and/or its equivalent fragments, homologs, and sequences that hybridize under highly stringent and/or medium stringent conditions to the specifically named nucleotide sequence) and/or the recited protein sequence (and/or its equivalent fragments, variants, and proteins of the same molecular weight), respectively. The composition may comprise an aqueous solution containing, for example, salts (e.g., NaCl), detergents (e.g., SDS), and other components (e.g., Denhardt's solution, dry milk, salmon sperm DNA, etc.).

The terms nucleotide sequence “comprising a particular nucleic acid sequence” and protein “comprising a particular amino acid sequence” and equivalents of these terms, refer to any nucleotide sequence of interest and to any protein of interest, respectively, that contain the particularly named nucleic acid sequence (and/or its equivalent fragments, homologs, and sequences that hybridize under highly stringent and/or medium stringent conditions to the specifically named nucleotide sequence) and the particularly named amino acid sequence (and/or its equivalent fragments, variants, and sequences of the about the same molecular weight), respectively. The invention does not limit the source (e.g., cell type, tissue, animal, etc.), nature (e.g., synthetic, recombinant, purified from cell extract, etc.), and/or sequence of the nucleotide sequence of interest and/or protein of interest. In one embodiment, the nucleotide sequence of interest and protein of interest include coding sequences of structural genes (e.g., probe genes, reporter genes, selection marker genes, oncogenes, drug resistance genes, growth factors, etc.).

### BRIEF DESCRIPTION OF THE INVENTION

The invention provides compositions comprising amino acid sequences that have cell killing activity, nucleic acid sequences encoding them, antibodies that specifically bind with them, and methods of using these compositions for increasing and/or reducing cell death, detecting cell death, diagnosing diseases associated with altered cell death, and methods for identifying test agents that alter cell death.

More particularly, the invention provides an activator of DNA fragmentation (ADF) having the amino acid sequence SEQ ID NO:7 (KAKAGAGSAT LSMAYAGARF VFSLVDAMNG KEGVVECSFV KSQETECTYF STPLLLGKKG IEKNLGIGKV SSFEEKMISD AIPELKASIK KGEDFVKTLK), and fragments of ADF mediating its activity, such as the “minimum activator of DNA fragmentation (“MADF”) sequence SEQ ID NO:6 (KAKAGAGSAT LSMAYAGARF VFSLVDAMNG KEGVVECSFV KSQETECTYF STPLLLGKKG IEKNLGIGKV SS). The invention also provides homologs of ADF and MADF that mediate ADF's and MADF's activity, for example those obtained by amino acid substitution. The invention also provides an ADF assay suitable for identification of molecules or ADF homologs having an activity substantially equal to ADF or inhibitors of this activity. Also provided are nucleotide sequences encoding ADF and any sequence hybridizing to this sequence under intermediate stringency. Also provided are antisense sequences complementary to ADF useful to inhibit ADF; RNAi, 21 bp, RNA fragments complementary to ADF useful to inhibit ADF; antibodies that bind ADF for diagnostic purposes; the use of ADF as a protein to induce cell death (such as by apoptosis); the use of ADF as a transgene to induce cell death (such as by apoptosis); the use of ADF to screen for modulators of cell death (such as by apoptosis); the use of ADF in fusion proteins containing enzymes, growth factors, antibodies, and other ligands for therapeutic or diagnostic purposes; all proteins identified in a protein-protein interaction trap assay that bind/interact with ADF; and molecules that inhibit interaction of DF with any other protein.

Thus, in one embodiment, the invention provides a conjugate comprising a cell death-inducing molecule (such as peptide) and a cell molecule-recognizing compound (such as a cell marker-recognizing compound). The cell death-inducing molecule comprises a mitochondrial protein or protein fragment. In one embodiment, the cell death-inducing molecule is of mammalian origin, such as of human or primate origin. The cell death-inducing molecule may comprise the “activator of DNA fragmentation” (ADF), a fragment of mitochondrial malate dehydrogenase (MDH). In one embodiment, the cell death-inducing molecule comprises a fragment of ADF, wherein the fragment comprises at least 8, preferably at least 10, amino acids derived from SEQ ID NO:7.

Also provided is a conjugate of a cell death-inducing molecule and a cell molecule-recognizing compound (such as a cell marker-recognizing compound) wherein the cell death-inducing molecule comprises an amino acid sequence that is a derivative of SEQ ID NO:7, comprising an amino acid stretch that is at least 80% identical in sequence to SEQ ID NO:7 over an amino acid stretch of at least 10 consecutive residues of SEQ ID NO:7, with or without insertions or deletions in the corresponding sequence of the cell death-inducing molecule. Alternatively, the derivative comprises an amino acid stretch that is at least 60% identical in sequence to SEQ ID NO:7 over an amino acid stretch of at least 12 consecutive residues of SEQ ID NO:7, with or without insertions or deletions in the corresponding sequence of the cell death-inducing molecule. In another alternative, the derivative comprises an amino acid stretch that is at least 40% identical in sequence to SEQ ID NO:7 over an amino acid stretch of at least 15 consecutive residues of SEQ ID NO:7, with or without insertions or deletions in the corresponding sequence of the cell death-inducing molecule. In a further alternative, the DNA encoding the cell death-inducing molecule can be hybridized to any DNA encoding SEQ ID NO:7, at 60 C at 1.5 M, 1.0 M, 0.75 M, or 0.5 M salt.

The invention also provides a DNA sequence encoding any of the conjugates of the invention, preferably wherein the conjugate further comprises an N-terminal signal peptide, as well as cells containing vectors that comprise these DNA sequences.

The invention additionally provides a conjugate comprising a cell death-inducing molecule and a cell molecule-recognizing compound (such as a cell marker-recognizing compound), wherein the cell death-inducing comprises a peptide chosen from Htra2/Omi, AIF (apoptosis-inducing factor), Smac/DIABLO, activated gelsolin, AP24 serine protease, any pro-apoptotic Bcl-2 family members, any intracellular nuclease, EndoG, Cytochrome C, Nix, Nip3, CIDE-B, and equivalent fragments thereof, variants thereof, and sequences of about the same molecular weight. Further provided is a conjugate of a cell death-inducing molecule and a cell molecule-recognizing compound (such as a cell marker-recognizing compound) wherein the cell death-inducing molecule comprises an amino acid sequence that comprises a derivative of any peptide in the first group of peptides of described above. In one embodiment the derivative comprises an amino acid stretch that is at least 80% identical in sequence to any cell death-inducing molecule described herein over an amino acid stretch of at least 10 consecutive residues, with or without insertions or deletions in the corresponding sequence of the cell death-inducing molecule. Alternatively, the derivative comprises an amino acid stretch that is at least 60% identical in sequence to any cell death-inducing molecule described herein over an amino acid stretch of at least 12 consecutive residues, with or without insertions or deletions in the corresponding sequence of the cell death-inducing molecule. In another alternative, the derivative comprises an amino acid stretch that is at least 40% identical in sequence to any cell death-inducing molecule described herein over an amino acid stretch of at least 15 consecutive residues, with or without insertions or deletions in the corresponding sequence of the cell death-inducing molecule. In yet another alternative, the DNA encoding the cell death-inducing molecule can be hybridized to any DNA encoding any cell death-inducing molecule of the first group of peptides described herein at 60 C at 1.5 M, 1.0 M, 0.75 M, or 0.5 M salt. In another alternative, the pro-apoptotic Bcl-2 family member is chosen from a group containing one or more of Bax, Bad, and Bid. In a further embodiment, the intracellular nuclease comprises one or more of EndoG, intracellular DNase I, DNase II, and caspase-activated DNase (CAD). More preferably, the nuclease comprises a fragment of CAD that is more resistant than full-lengthCAD to inhibition by ICAD, and wherein the conjugate also comprises a nuclear localization signal. Yet more preferred, is that the fragment of CAD that is more resistant than full-length CAD to inhibition by ICAD is a fragment of CAD that comprises the nuclease domain but not the CAD domain.

In one embodiment, the conjugate comprises more than one copy of the cell death-inducing molecule, and/or the cell molecule-recognizing compound (such as a cell marker-recognizing compound). In one embodiment, the conjugation of the cell death-inducing molecule and the cell molecule-recognizing compound is accomplished using a chemical bioconjugation reaction. Preferably, the bond between the cell death-inducing molecule and the cell molecule-recognizing compound is a cleavable bond that can be cleaved when the conjugate enters a cell. More preferably, the cleavable bond is an ester that can be cleaved by an esterase inside of a cell, or is a disulfide bond, such as a disulfide bond between two cysteine residues, one on the cell death-inducing molecule and one on the cell molecule-recognizing compound. Alternatively, the cleavable bond is a peptide bond that can be cleaved by an intracellular protease. In another embodiment, the conjugation of the cell death-inducing molecule and the cell molecule-recognizing compound is done genetically to form a fusion protein. In one embodiment, the conjugation is at the N-terminus and/or C-terminus of the cell molecule-recognizing compound.

In one embodiment, the cell molecule-recognizing compound (such as a cell marker-recognizing compound) of the conjugate comprises a monoclonal antibody (including an antibody fragment), such as those identified from an antibody fragment library, phage display, or phagemid display. In a particular embodiment, the cell molecule-recognizing compound is a cancer cell-specific marker, such one that recognizes liver cancer cells, including hepatocellular carcinoma cells, and is more preferably an antibody, an antibody fragment, a F(ab)′2, a Fab′, a Fab, or a single-chain Fv (scFv). In a further embodiment, the cell molecule-recognizing compound specifically recognizes a liver cancer cell marker and comprises the heavy chain variable region from an antibody selected from a group of rodent monoclonal antibodies containing one or more of Hepama-1, anti-PLC1, anti-PLC2, K-PLC1, K-PLC2, K-PLC2, 49-D6, 7-E10, 34-A4,26-A10, 34-B9,79-C8,16-E10, 5D3, 5C3, 2C6, a-AFP, HP-1, hHP-1, mAb 95YPC2/38.8, P215457, PMM4E9917, HAb25, HAb27, KY-1, KY-2, KY-3, 9403 Mab, KM-2, S1, 9B2, IB1, A9-84, SF-25, AF-10, XF-8, AF-20, a-hIRS-1, FB-50, SF 31, SF 90, 2A3D2, and 2D11E2. Also contemplated are cell marker-recognizing compounds that specifically recognize the same antigen as any of these antibodies. Alternatively, the cell death-inducing molecule of the conjugate comprises ADF, including a fragment thereof, and the cell molecule-recognizing compound specifically recognizes the same antigen as the Hepama-1 monoclonal antibody, such as a fragment of the Hepama-1 monoclonal antibody, a humanized version of the Hepama-1 monoclonal antibody, and a humanized version of a fragment of the Hepama-1 monoclonal antibody. Preferably, the cell molecule-recognizing compound and the cell death-inducing molecule are genetically fused. In a further embodiment, the cell molecule-recognizing compound specifically recognizes the same antigen as any antibody from the above group of mouse monoclonal antibodies. Alternatively, the cell molecule-recognizing compound can bind to liver cancer cells competitively with any of the above described antibodies. Also provided is conjugate wherein the cell molecule-recognizing compound and any antibody from the above group of mouse monoclonal antibodies can bind simultaneously to the same cell molecule, as shown by immunoprecipitation of the cell molecule-recognizing compound by any antibody from the group of the above mouse monoclonal antibodies in the presence of solubilized cell proteins from liver cancer cells. Preferably, the cell molecule-recognizing compound is a cell marker-recognizing compound that specifically binds to a liver cancer cell protein and wherein the molecular weight of the protein is about 43,000 daltons, and more preferably, the liver cell marker is a glycoprotein.

In another embodiment, the invention provides a conjugate between a cell-killing agent and a cell molecule-recognizing compound (such as a cell marker-recognizing compound), wherein the cell molecule-recognizing compound binds to the same antigen as the Hepama-1 monoclonal antibody with higher affinity than the Hepama-1 antibody. The cell molecule-recognizing compound may be obtained through the use of monoclonal antibody technology, such as rabbit monoclonal antibody technology, obtained through the use of mRNA display or ribosome display, or obtained through a. determining the identity of the Hepama-1 antigen, b. purifying the Hepama-1 antigen or fragment thereof, and c. creating a binding agent against the purified Hepama-1 antigen or fragment thereof. Alternatively, the protein-binding agent is obtained through a. determining the identity of the Hepama-1 antigen, b. synthesizing a peptide comprising the extracellular peptide portion of the Hepama-1 antigen, c. creating a binding agent against the peptide, and d. confirming that the cell molecule-recognizing compound against the peptide can recognize the Hepama-1 antigen on hepatocellular carcinoma cells with a higher affinity than the Hepama-1 antibody. In one embodiment, the cell-killing agent comprises one or more of a radionuclide, and a protein-based toxin such as ADF. Preferably, the conjugates of the invention that contain an antibody have been subjected to a complete or partial humanization or de-immunization process, or are chimeras between human and murine antibody sequences.

In an alternative embodiment, the cell molecule-recognizing compound of the conjugate is a ligand of a cell receptor, such as a cancer-specific cell receptor (such as those in Tables 1 and 2). Alternatively, the ligand is a growth factor, such as epidermal growth factor, insulin-like growth factor, fibroblast growth factor or vascular endothelial growth factor.

In yet another embodiment, the cell molecule-recognizing compound (such as a cell marker-recognizing compound) of the conjugate comprises a nucleic acid aptamer, a peptide, a constrained peptide, a single domain antibody, a diabody, and/or a partially randomized protein based on a known structural motif, on the structure of a lens crystalline or of a domain of fibronectin. Preferably, where the cell molecule is a cell surface marker, once attached to the corresponding cell surface marker on a cell, the conjugates of the invention are internalized into the cell, such as where the conjugate contains cell internalization signal as exemplified by a polycationic peptide (for example, a polycationic peptide comprising one or more of the HIV Tat protein, 6-9 arginine moieties, the antennapedia protein, and human HOX protein). In another embodiment, the conjugates of the invention contain a nuclear localization signal, such as the amino acid sequence (SEQ ID NO:62) PKKKRKV.

The invention also provides methods for reducing symptoms of a disease (such as cancer, and in particular, liver cancer) by administering any of the conjugate described herein to a mammalian subject, such as a human

The invention additionally provides antibody that specifically binds to ADF, preferably a monoclonal antibody, and more preferably an antibody that binds to uncleaved mitochondrial malate dehydrogenase with substantially lower affinity than to ADF.

Also provided is a method for characterizing the apoptotic state of cells by measuring the abundance of ADF in their cytoplasm, such as by using one or more antibodies or other specific ADF-binding agents to quantify the levels of ADF in the cytoplasm of cells. Further provided are methods of characterizing or diagnosing a patient based on the abundance of ADF in blood, including blood-derived specimens (such as plasma, platelets, etc.).

The invention also provides methods for identifying factors in biological samples that cause death in apoptosis-resistant or necrosis-resistant eukaryotic cells comprising the steps of a. incubation of cells, cell extracts or isolated nuclei from the apoptosis-resistant cells with the biological samples to assay for the presence of cell death-inducing factors and, wherein the number of different factors being exposed to the cells, cell extracts or isolated nuclei is greater than 100, and b. in the cases where the biological sample is observed to possess cell death-inducing activity, analysis of the sample that possesses cell death-inducing activity to determine the identity of the component with the activity. Preferably, the apoptosis-resistant or necrosis-resistant eukaryotic cells are apoptosis-resistant, and wherein the apoptosis resistance is a result of Bcl-2 overexpression.

In one embodiment, the biological samples comprise complex mixtures of biological components, and the analysis of the samples that possesses cell death-inducing activity to determine the identity of the component with the activity comprises the steps of a. fractionation of the extract, and b. testing fractionated extracts for cell death-inducing activity and c. for fractions containing cell death-inducing activity, determination of the component(s) responsible for inducing cell death. In a further embodiment, the biological samples comprise cell extracts or media from cells in which proteins have been expressed from a DNA construct that was introduced to the cells, and where the analysis of the sample that possesses cell death-inducing activity to determine the identity of the component with the activity comprises the determination of the DNA sequence of the DNA constructs associated with the sample that possesses cell death-inducing activity. Alternatively, the method further comprises the steps of a. Creating a library of the DNA constructs and b. Introducing the library of the DNA constructs into the cells and c. Screening extracts or media from the cells into which the DNA constructs have been introduced, the screening comprises an assay for cell death-induction and d. Determining the sequence of the DNA construct that was present in the cells that correspond to the cell extracts or media that induce cell death. More preferably, each extract or media from the cells into which the DNA constructs have been introduced is derived from cells carrying a single DNA construct. Alternatively, each extract or media from the cells into which the DNA constructs have been introduced is derived from cells carrying a group of DNA constructs, and wherein an assay for the induction of cell death is used to identify cell death-inducing groups of DNA constructs, and wherein further screening of a group of cell death-inducing DNA constructs is then performed on cells carrying single DNA constructs from the group of cell death-inducing DNA constructs, to identify which of the DNA constructs encodes proteins responsible for the induction of cell death. While not intending to limit the invention to any mechanism, in one embodiment, the mechanism of cell death is apoptosis, necrosis, aponecrosis and/or autophagic degeneration. In another embodiment, the eukaryotic cell extracts are from untreated cells, from cells treated with UV radiation or other apoptosis, necrosis, aponecrosis-inducing agent, from human cells, and/or are extracts enriched in components from cellular organelles, such as mitochondria. The factors in the biological samples that may be identified in accordance with the invention's methods may be peptides, polypeptides, proteins, lipids, oligosaccharides, small molecules, etc. The assay for the presence of cell death-inducing factors includes a DNA fragmentation assay.

In another embodiment, the invention provides methods of identifying gene products that can cause cell death in apoptosis-resistant cells, comprising the steps of: a. Introducing DNA into an apoptosis-resistant host cell, the DNA comprising all cis-acting sequences necessary to express a gene under the control of an induction system, b. Inducing the expression of the gene and c. Monitoring the host cell for indications of death. Preferably, the method further comprises the steps of d. Creating a library of the DNA constructs and e. Introducing the library of the DNA constructs into the host cells and f. Determining the identities of the apoptosis-inducing gene products by determining the identity of the DNA constructs that cause death in the apoptosis-resistant host cells.

Also provided are methods for identifying compounds that suppress cell death (such as by apoptosis) in cells, comprising the steps of a. Adding a molecule comprising ADF to cells or cellular extracts and assaying these extracts for markers of apoptosis and b. In addition to adding the molecule to the cells or cellular extracts, also adding a compound and c. Assaying for the inhibition of apoptosis by the compound. In another embodiment, the invention provides a method for identifying compounds that promote cell death (such as by apoptosis), comprising the steps of a. Identifying an interaction molecule that binds to ADF in cells and b. Identifying compounds that interact with the interaction molecule and c. Assaying these the compounds that can interact with the interaction molecule for their ability to promote cell death (such as by apoptosis). Preferably, the identification of molecules that bind to ADF is accomplished using the two hybrid system, phage display, or other combinatorial biology methods, or using a pull-down assay followed by mass-spectrometry of pulled-down molecules, or by an in-gel or on-filter binding of ADF to electrophoretically separated cell extracts.

In a further embodiment, the invention provides a method for identifying compounds that inhibit cell death (such as by apoptosis), comprising the steps of a. Identifying an interaction molecule that binds to ADF in cells and b. Identifying compounds that interact with the interaction molecule and c. Assaying these the compounds that can interact with the interaction molecule for their ability to inhibit ADF-induced cell death (such as by apoptosis). Preferably, the identification of molecules that bind to ADF is accomplished using the two hybrid system, phage display, or other combinatorial biology methods, or using a pull-down assay followed by mass-spectrometry of pulled-down molecules, or by an in-gel or on-filter binding of ADF to electrophoretically separated cell extracts.

Also provided is conjugate of a cell death-inducing molecule and a cell molecule-recognizing compound (such as a cell marker-recognizing compound) wherein the cell death-inducing molecule comprises a peptide that is at least 80% identical in sequence to any peptide identified according to any of the invention's methods, over an amino acid stretch of at least 10 consecutive residues in the sequence of the peptide identified according to any of the invention's methods, with or without insertions or deletions in the corresponding sequence of the cell death-inducing molecule. In one embodiment, the invention provides a conjugate of a cell death-inducing molecule and a cell marker-recognizing compound wherein the cell death-inducing molecule comprises a peptide that is at least 60% identical in sequence to any peptide identified according to any of the invention's methods, over an amino acid stretch of at least 12 consecutive residues in the sequence of the peptide identified according to any of the invention's methods, with or without insertions or deletions in the corresponding sequence of the cell death-inducing molecule. Further provided is a conjugate of a cell death-inducing molecule and a cell marker-recognizing compound wherein the cell death-inducing molecule comprises a peptide that is at least 40% identical in sequence to any peptide identified according to any of the invention's methods, over an amino acid stretch of at least 15 consecutive residues in the sequence of the peptide identified according to any of the invention's methods, with or without insertions or deletions in the corresponding sequence of the cell death-inducing molecule. The invention also provides conjugate of a cell death-inducing molecule and a cell marker-recognizing compound wherein the DNA encoding the cell death-inducing molecule can be hybridized to any DNA encoding any polypeptide identified according to any of the invention's methods, at 60° C. at 1.5 M, 1.0 M, 0.75 M, or 0.5 M salt.

The invention also provides a method for killing specific cells in a human or an animal, the specific cells bearing a cell marker, comprising steps of a. Generating a conjugate of a cell death-inducing molecule and a cell marker-recognizing compound, wherein the cell death-inducing molecule comprises a peptide chosen from one or more of Htra2/Omi, AIF (apoptosis-inducing factor), Smac/DIABLO, activated gelsolin, AP24 serine protease, any pro-apoptotic Bcl-2 family members, any intracellular nuclease, EndoG, Cytochrome C, Nix, Nip3, CIDE-B, any compound identified according to any of the invention's methods, any peptide derived from fragmentation of any peptide in the group, or that has at least 80% sequence identity over at least a 10 amino acid stretch to any peptide belonging to the group, and b. Administering the compound to a human or animal, such that the conjugate will have access to cells displaying the cell marker (such as a cell surface marker) and c. Allowing the conjugate to contact the cells displaying the cell marker and thereafter allowing the conjugate to be internalized by the cells displaying the cell markers, and d. Allowing the toxic compound to cause the cells displaying the cell marker to be killed. In one embodiment, the administration comprises injection of the conjugate into the human or animal, or administration of DNA constructs that cause expression of the conjugates, such that the expressed conjugates will have access to the cells displaying the cell markers. In one embodiment, the cell marker is specific to cancer cells, such as hepatocellular carcinoma cells. Preferably, the conjugate comprises ADF, including a fragment thereof, and an antibody or antibody fragment selected from the group of mouse monoclonal antibodies described herein. Preferably, the conjugate comprises ADF, including a fragment thereof, and a cell marker-recognizing compound that recognizes the same antigen as the Hepama-1 monoclonal antibody, and more preferably, the cell marker-recognizing compound is a humanized or de-immunized version of the Hepama-1 monoclonal antibody, including a fragment thereof. In one embodiment, the human or other animal is also treated with an anti-cancer chemotherapeutic agent, radiation therapy, or protein-based therapy, including antibody-based therapies. In one embodiment, the anti-cancer chemotherapeutic agent is selected from a group of compounds containing one or more of 5-Fluorouracil, Leucovorin, Tomudex, Mitomycin C, CPT-11, and 3-bromopyruvate.

The invention also provides a method of killing specific cells in a human or an animal, the specific cells bearing a cell marker (such as cell surface marker), comprising steps of a. delivering a biotinylated cell marker-recognizing compound to a human or animal, such that the biotinylated cell marker recognizing compound has access to cells bearing the cell marker, such that the biotinylated cell marker-recognizing compound will contact the cells, b. delivering a toxin conjugate of a biotin-binding protein and a cell death-inducing molecule, wherein the cell death-inducing molecule comprises a peptide chosen from one or more of Htra2/Omi, AIF (apoptosis-inducing factor), Smac/DIABLO, activated gelsolin, AP24 serine protease, any pro-apoptotic Bcl-2 family members, any intracellular nuclease, EndoG, Cytochrome C, Nix, Nip3, CIDE-B, any compound identified according to any of the invention's methods, or any peptide derived from fragmentation of any peptide in the group, or that has at least 80% sequence identity over at least a 10 amino acid stretch to any peptide belonging to the group, c. Allowing the toxin conjugate to contact the biotinylated cell marker-recognizing compound bound to the cells, such that the cell death-inducing molecule becomes indirectly attached to the cells, d. Allowing the toxin conjugate indirectly attached to the cells to be internalized by the cells, and e. Allowing the toxic compound to cause the cells to be killed.

Further provided is a method of killing specific cells in a human or an animal, the specific cells bearing a cell marker (such as a cell surface marker), comprising steps of a. delivering a targeting conjugate of a biotin-binding protein and a cell marker-recognizing compound to a human or animal, such that the cell marker recognizing compound has access to cells bearing the cell marker, such that the targeting conjugate will contact the cells, b. delivering a biotinylated cell death-inducing molecule, wherein the cell death-inducing molecule comprises a peptide chosen from one or more of Htra2/Omi, AIF (apoptosis-inducing factor), Smac/DIABLO, activated gelsolin, AP24 serine protease, any pro-apoptotic Bcl-2 family members, any intracellular nuclease, EndoG, Cytochrome C, Nix, Nip3, CIDE-B, any compound identified according to any of the invention's methods, or any peptide derived from fragmentation of any peptide in the group, or that has at least 80% sequence identity over at least a 10 amino acid stretch to any peptide belonging to the group, c. Allowing the biotinylated cell death-inducing molecule to contact the targeting conjugate bound to the cells, such that the cell death-inducing molecule becomes indirectly attached to the cells, d. Allowing the biotinylated cell death-inducing molecule indirectly attached to the cells to be internalized by the cells and e. Allowing the biotinylated cell death-inducing molecule to cause the cells to be killed.

The invention also provides therapeutic interventions based on any of the invention's methods, as well as pharmaceutical compositions useful in the treatment of carcinomas comprising a pharmaceutically effective amount of the any of the conjugates of the invention and an acceptable carrier, and further still, provides methods of treating carcinomas in vivo comprising administering to a patient a pharmaceutically effective amount of a composition containing any of the invention's conjugates.

### DETAILED DESCRIPTION OF THE INVENTION

The invention provides compositions comprising amino acid sequences that have cell killing activity, nucleic acid sequences encoding them, antibodies that specifically bind with them, and methods of using these compositions for increasing and/or reducing cell death, detecting cell death, diagnosing diseases associated with altered cell death, and methods for identifying test agents that alter cell death.

The invention is further described under A. Amino Acid Sequences Of The Invention, B. Conjugates Comprising The Invention's Sequences, C. Nucleic Acid Sequences Of the Invention, D. Vectors And Cells, E. Antibodies Specific for ADF, F. Methods For Killing Cells, G. Methods For Reducing Cell death, H. Methods For Detecting Apoptosis, I. Methods For Identifying Agents That Alter Cell Death, J. Methods For Identifying Molecules That Increase Cell Death, and K. Methods For Identifying Molecules That Reduce Cell Death.

A. Amino Acid Sequences Of The Invention

The invention provides a composition comprising an amino acid sequence that comprises one or more of (1) a portion of SEQ ID NO:4 (human mitochondrial malate dehydrogenase) comprising the minimum activator of DNA fragmentation protein SEQ ID NO:6. In one embodiment, the amino acid sequence has activity chosen from one or more of DNA nuclease activity and cell-killing activity. The invention's sequences embody the discovery of a fragment of mitochondrial malate dehydrogenase that is released during apoptosis and that activates nuclear DNA fragmentation.

An advantage of the amino acid sequences of the invention is that sequences that are derived from humans are non-immunogenic, and thus do not require (although they may be) modified by PEGylation in order to reduce the immune response. This is advantageous since PEGylation may result in inactivation of the protein, increase the molecular weight of the protein thereby reducing its penetration into cells and consequently also reducing its biological activity (e.g., DNA nuclease activity and/or cell-killing activity) in cells.

The invention's MDH portions, MADF, and ADF are useful as cell-killing molecules. The terms “cell killing” and “cell death,” refer to programmed and/or unprogrammed dying of cells by any mechanism, such as by apoptosis, necrosis, aponecrosis, autophagic degeneration, etc. Thus, “killing cells,” “cell death-inducing,” “increasing cell death,” “cell death activity,” “cytotoxic activity,” and grammatical equivalents refer to increasing the number of dead cells and/or reducing the number of viable and/or dividing cells by any mechanism, including (but not limited to) increasing apoptosis, increasing necrosis, increasing aponecrosis, increasing autophagic degeneration, reducing viability, reducing the rate of cell division, etc.

Methods for detecting and quantifying cell death are known in the art, such as for detecting apoptosis. The term “apoptosis” means non-necrotic cell death that takes place in metazoan animal cells following activation of an intrinsic cell suicide program. Apoptosis is a normal process in the development and homeostasis of metazoan animals. Apoptosis involves characteristic morphological and biochemical changes, including cell shrinkage, zeiosis, or blebbing, of the plasma membrane, and nuclear collapse and fragmentation of the nuclear chromatin, at intranucleosomal sites. During apoptosis, cells undergo various changes that result in the eventual lysis of the cell into apoptotic bodies which are then typically phagocytosed by other cells. One of skill in the art appreciates that reducing the level of apoptosis results in increased cell survival, without necessarily (although it may include) increasing cell proliferation. Accordingly, as used herein, the terms “increase apoptosis” and “reduce survival” are equivalent. As used herein, the terms “reduce apoptosis” and “increase survival” are equivalent. Apoptosis may be determined using methods known in the art, such as those disclosed herein, including measuring the cells' display of increased annexin-V binding to phosphatidylserine in plasma membranes, an early indicator of apoptosis, by live microscopy, or cell sorting analysis (FACS) for the transfection indicator green fluorescent protein and annexin-V. Also, apoptosis may confirmed by nuclear staining with Hoechst 33342. The terms “apoptosis activity” as used herein refers to the ability to cause and/or increase apoptosis.

Other methods for detecting and quantifying cell death include detecting and quantifying cell proliferation such as by incubating the cells with bromodeoxyuride (BrdU), which is incorporated into the DNA of dividing cells, followed by detecting BrdU incorporation into DNA by immunohistochemistry. The proliferation index may be calculated as the percentage of BrdU-positive target cells per total cells in the samples. Alternatively, the level cell proliferation may be determined by staining tissue sections with antibodies to proliferating cell nuclear antigen (PCNA), which is a marker for cells at the S phase of the cell cycle, followed by counting the number of PCNA positive cells in the tissue.

The amino acid sequences of the invention may be “endogenous” or “heterologous” (i.e., “foreign”). The terms “endogenous” and “wild type” when in reference to a peptide sequence and nucleotide sequence refers to a sequence which is naturally found in the cell into which it is introduced so long as it does not contain some modification relative to the naturally-occurring sequence. The term “heterologous” refers to a sequence which is not endogenous to the cell or virus into which it is introduced. For example, heterologous DNA includes a nucleotide sequence which is ligated to, or is manipulated to become ligated to, a nucleic acid sequence to which it is not ligated in nature, or to which it is ligated at a different location in nature. Heterologous DNA also includes a nucleotide sequence which is naturally found in the cell into which it is introduced and which contains some modification relative to the naturally-occurring sequence. Generally, although not necessarily, heterologous DNA encodes heterologous RNA and heterologous proteins that are not normally produced by the cell or virus into which it is introduced. Examples of heterologous DNA include reporter genes, transcriptional and translational regulatory sequences, DNA sequences which encode selectable marker proteins (e.g., proteins which confer drug resistance), etc.

The terms “naturally occurring” and “wild type” as used herein when applied to a molecule or composition (such as nucleotide sequence, amino acid sequence, cell, apoptotic blebs, etc.), mean that the molecule or composition can be found in nature and has not been intentionally modified by man. For example, a naturally occurring polypeptide sequence refers to a polypeptide sequence that is present in an organism that can be isolated from a source in nature, wherein the polypeptide sequence has not been intentionally modified by man.

In one embodiment, the invention also provides an amino acid sequence that comprises a portion of the exemplary human mitochondrial malate dehydrogenase (MDH) SEQ ID NO:4 (GenGenBank: NP_{—}005909) (MLSALARPASAALRRSFSTSAQNNAKVAVLGASGGIGQPLSLLL KNSPLVSRLTLYDIAHTPGVAADLSHIETKAAVKGYLGPEQLPDCLKGCDVVVIP AGVPRKPGMTRDDLFNTNATIVATLTAACAQHCPEAMICVIANPVNSTIPITAEVF KKHGVYNPNKIFGVTTLDIVRANTFVAELKGLDPARVNVPVIGGHAGKTIIPLISQ CTPKVDFPQDQLTALTGRIQEAGTEVVKAKAGAGSATLSMAYAGARFVFSLVDA MNGKEGVVECSFVKSQETECTYFSTPLLLGKKGIEKNLGIGKVSSFEEKMISDAIP ELKASIKKGEDFVKTLK) comprising a “minimum activator of DNA fragmentation” (“MADF”) sequence SEQ ID NO:6 (KAKAGAGSAT LSMAYAGARF VFSLVDAMNG KEGVVECSFV KSQETECTYF STPLLLGKKG IEKNLGIGKV SS). In a most preferred embodiment, the amino acid sequence comprises the human “activator of DNA fragmentation” (“ADF”) SEQ ID NO:7 (KAKAGAGSAT LSMAYAGARF VFSLVDAMNG KEGVVECSFV KSQETECTYF STPLLLGKKG IEKNLGIGKV SSFEEKMISD AIPELKASIK KGEDFVKTLK).

In another embodiment, the invention also provides an amino acid sequence that comprises a portion of the exemplary pig mitochondrial malate dehydrogenase (MDH) SEQ ID NO:1 (GenBank: P00346) (AKVAVLGASG GIGQPLSLLL KNSPLVSRLT LYDIAHTPGV AADLSHIETR ATVKGYLGPE QLPDCLKGCD VVVIPAGVPR KPGMTRDDLF NTNATIVATL TAACAQHCPD AMICIISNPV NSTIPITAEV FKKHGVYNPN KIFGVTTLDI VRANAFVAEL KGLDPARVSV PVIGGHAGKT IIPLISQCTP KVDFPQDQLS TLTGRIQEAG TEVVKAKAGA GSATLSMAYA GARFVFSLVD AMNGKEGVVE CSFVKSQETD CPYFSTPLLL GKKGIEKNLG IGKISPFEEK MIAEAIPELK ASIKKGEEFV KNMK) comprising a “minimum activator of DNA fragmentation” (“MADF”) sequence SEQ ID NO:2 (KAKAGA GSATLSMAYA GARFVFSLVD AMNGKEGVVE CSFVKSQETD CPYFSTPLLL GKKGIEKNLG IGKISP).

Reference herein to any specifically named protein and/or its sequence identifier (such as MDH, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, HIV Tat protein, etc.) refers to any and all equivalent fragments thereof, variants thereof, and sequences of about the same molecular weight. In one embodiment, equivalent proteins have at least one of the biological activities (such as those disclosed herein and/or known in the art) of the specifically named protein, wherein the biological activity is detectable by any method.

Reference herein to any specifically named protein or its sequence identifier also includes equivalent polypeptide sequences that have about the same molecular weight as the specifically named protein. The term “about the same molecular weight” means having a molecular weight that is +/−5%, +/−10%, +/−1-15%, and/or +/−20% of the molecular weight of the polypeptide in issue. The molecular weight may be determined by, for example, denaturing polyacrylamide gel electrophoresis.

The terms “fragment” and “portion” when in reference to a protein (such as MDH, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, HIV Tat protein, etc.) refers to a portion of that protein that range in size from an exemplary 4, 8, 10, 20, 30, and 50 contiguous amino contiguous amino acid residues to the entire amino acid sequence minus one amino acid residue. Thus, a polypeptide comprising “at least a portion of an amino acid sequence” comprises from four (4) contiguous amino acid residues of the amino acid sequence to the entire amino acid sequence.

Exemplary peptide sequence fragments within the scope of the invention include, without limitation, MDH (SEQ ID NOs:1, 4), MADF (SEQ ID NOs:2, 6) ADF (SEQ ID NOs:3, 7), Htra/Omi (SEQ ID NO:8), apoptosis inducing factor (SEQ ID NO:10), Smac/DIABLO (SEQ ID NO:12), EndoG (SEQ ID NO:24), Cytochrome C (SEQ ID NO:72), Nix (SEQ ID NO:74), Nip3 (SEQ ID NO:76), CIDE-B (SEQ ID NO:78), gelsolin (SEQ ID NO:14), Bcl-2 (SEQ ID NO:16), Bax (SEQ ID NO:18), Bad (SEQ ID NO:20), Bid (SEQ ID NO:22), caspase-activated DNase (SEQ ID NO:26), DNase I (SEQ ID NO:40), DNase II (SEQ ID NO:42), inhibitor of CAD nuclease (SEQ ID NO:28), epidermal growth factor (SEQ ID NO:30), vascular endothelial growth factor (SEQ ID NO:32), lens crystalline protein (SEQ ID NO:34), antennapedia protein (SEQ ID NO:36), fibronectin type 1 (SEQ ID NO:38), human HOX protein (SEQ ID NO:45), insulin-like growth factor (SEQ ID NO:46), fibroblast growth factor (SEQ ID NO:48), and HIV Tat protein (SEQ ID NO:50), that lack (1) one N-terminal amino acid, (2) two N-terminal amino acids, (3) three N-terminal amino acids, (4) four N-terminal amino acids, (5) five N-terminal amino acids, (6) six N-terminal amino acids, (6) six N-terminal amino acids, (7) one C-terminal amino acid, (8) two C-terminal amino acids, (9) three C-terminal amino acids, (10) four C-terminal amino acids, (11) five C-terminal amino acids, (12) six C-terminal amino acids, (13) seven C-terminal amino acids, (13) one N-terminal amino acid and one C-terminal amino acid, (14) two N-terminal amino acids and one C-terminal amino acid, (15) three N-terminal amino acids and one C-terminal amino acid, (16) four N-terminal amino acids and one C-terminal amino acid, (17) one N-terminal amino acid and two C-terminal amino acids, (18) one N-terminal amino acid and three C-terminal amino acids, (19) one N-terminal amino acid and four C-terminal amino acids, (20) one N-terminal amino acid and five C-terminal amino acids, (21) one N-terminal amino acid and six C-terminal amino acids, (22) one N-terminal amino acid and seven C-terminal amino acids, (23) two N-terminal amino acids and two C-terminal amino acids, (24) two N-terminal amino acids and three C-terminal amino acids, (25) three N-terminal amino acids and four C-terminal amino acids, (26) one N-terminal amino acid and four C-terminal amino acids, (27) two N-terminal amino acids and six C-terminal amino acids, (28) three N-terminal amino acids and two C-terminal amino acids, (29) six N-terminal amino acid and five C-terminal amino acids, and (30) five N-terminal amino acid and seven C-terminal amino acids.

Protein fragments may be produced by methods known in the art, such as by chemical fragmentation of a precursor protein. Alternatively, protein fragments may be produced by recombinant techniques involving creating a DNA sequence that encodes the desired protein fragment, and expressing it in a cell.

Sequences that are equivalent to one or more of the sequences discolsed herein (such as MDH portions, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, and HIV Tat protein), and that are within the scope of the invention include variants of these sequences. The terms “variant” and “homolog” of a protein as used herein refers to an amino acid sequence which differs by insertion, deletion, and/or conservative substitution of one or more amino acids from the protein of which it is a variant. The term “conservative substitution” of an amino acid refers to the replacement of that amino acid with another amino acid which has a similar hydrophobicity, polarity, and/or structure.

Preferably, the amino acid substitution does not substantially alter the biological activity of the molecule. Typically, conservative amino acid substitutions involve substitution of one amino acid for another amino acid with similar chemical properties (e.g., charge or hydrophobicity). For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

The following groups contain amino acids that are typical conservative substitutions for one another: i. Alanine (A), Serine (S), Threonine (T); ii. Aspartic acid (D), Glutamic acid (E); iii. Asparagine (N), Glutamine (Q); iv. Arginine (R), Lysine (K); v. Isoleucine (I), Leucine (L), Methionine (M), Valine (V); vi. Phenylalanine (F), Tyrosine (Y), Tryptophan (W); and vii. Alanine (A), Valine (V).

Guidance in determining which and how many amino acid residues may be substituted, inserted or deleted without abolishing biological and/or immunological activity may be found using computer programs well known in the art, for example, DNAStar™ software. In one embodiment, the sequence of the variant has at least 95% identity, at least 90% identity, at least 85% identity, at least 80% identity, at least 75% identity, at least 70% identity, and/or at least 65% identity with the sequence of the protein in issue.

Based on the above and other principles, exemplary variants of the human MDH (SEQ ID NO:4) (GenGenBank: NP_{—}005909) include the pig MDH (SEQ ID NO:1) (GenBank: P00346) encoded by the exemplary nucleic acid sequence SEQ ID NO:2.

Exemplary variants of human MADF include, without limitation, the pig minimum MADF (SEQ ID NO:2) (GenBank: P00346).

Exemplary variants of human ADF SEQ ID NO:7 include, without limitation, SEQ ID NO:3 from the pig MDH. Other exemplary variants of ADF have at least 40% identity over an amino acid stretch of at least 15 consecutive residues, preferably 60% identity over an amino acid stretch of at least 12 consecutive residues, and most preferably 80% identity over an amino acid stretch of at least 10 consecutive residues, of SEQ ID NO.7. Yet other exemplary variants of the ADF SEQ ID NO:7 (KAKAGAGSAT LSMAYAGARF VFSLVDAMNG KEGVVECSFV KSQETECTYF STPLLLGKKG IEKNLGIGKV SSFEEKMISD AIPELKASIK KGEDFVKTLK), include SEQ ID NO:7 in which glycine at one or more of Amino acid positions 5, 7, 17, 30, 33, 57, 60, 66, 68, 92 is replaced independently with any one of alanine, valine, leucine, isoleucine, serine, and threonine; alanine at one or more of amino acid positions 2, 4, 6, 9, 14, 16, 18, 27, 81, 87 is replaced independently with any one of glycine, valine, leucine, isoleucine, serine, and threonine; valine at one or more of amino acid positions 21, 25, 34, 35, 40, 70, 96 is replaced independently with any one of glycine, alanine, leucine, isoleucine, serine, and threonine; leucine at one or more of amino acid positions 11, 24, 54, 55, 56, 65, 85, 99 is replaced independently with any one of glycine, alanine, valine, isoleucine, serine, and threonine; isoleucine at one or more of amino acid positions 61, 67, 78, 82, 89 is replaced independently with any one of glycine, alanine, valine, leucine, serine, and threonine; serine at one or more of amino acid positions 8, 12, 23, 38, 42, 51, 71, 72, 79, 88 is replaced independently with any one of glycine, alanine, valine, leucine, isoleucine, and threonine; threonine at one or more of amino acid positions 10, 45, 48, 52, 98 is replaced independently with any one of glycine, alanine, valine, leucine, isoleucine, and serine; phenylalanine at one or more of amino acid positions 20, 22, 39, 50, 73, 95 is replaced independently with any one of tyrosine, and tryptophan; tyrosine at one or more of amino acid positions 15, 49 is replaced independently with any one of phenylalanine and tryptophan; cysteine at one or more of amino acid positions 37, 47 is replaced independently with methionine; methionine at one or more of amino acid positions 13, 28, 77 is replaced independently with cysteine; asparagine at one or more of amino acid positions 29, 64 is replaced independently with glutamine; glutamine at amino acid position 43 is replaced independently with asparagine; aspartic acid (aspartate) at one or more of amino acid positions 26, 80, 94 is replaced independently with glutamic acid (glutamate); glutamic acid (glutamate) at one or more of amino acid positions 32,36,44,46,62,74,75,84,93 is replaced independently with aspartic acid (aspartate); lysine at one or more of amino acid positions 1,3,31,41,58,59,63,69,76,86,90,91,97,100 is replaced independently with any one of arginine and histidine; and arginine at one or more of amino acid position 19 is replaced independently with any one of lysine and histidine.

Thus, with reference to the amino acid sequences of the invention, such as SEQ ID NO:1 (GenBank: P00346), SEQ ID NO:2 (GenBank: P00346), SEQ ID NO:3 (GenBank: P00346), SEQ ID NO:4 (GenBank: NP_{—}005909), SEQ ID NO:6 (GenBank: NP_{—}005909), SEQ ID NO:7 (GenBank: NP_{—}005909), SEQ ID NO:8 (GenGenBank: NM_{—}145074), SEQ ID NO:10 (GenBank: AF100928), SEQ ID NO:12 (GenBank: AF298770), SEQ ID NO:14 (GenBank: BC026033), SEQ ID NO:16 (Bcl-2 GenBank: M14745), SEQ ID NO:18 (GenBank: NM_{—}138764), SEQ ID NO:20 (GenBank:AF031523), SEQ ID NO:22 (GenBank: AF250233), SEQ ID NO:24 (GenBank: NM_{—}004435), SEQ ID NO:26 (GenBank: AB013918), SEQ ID NO:28 (GenBank: BC007112), SEQ ID NO:30 (GenBank: NM_{—}001963), SEQ ID NO:32 (GenBank: AY047581), SEQ ID NO:34 (GenBank: NM_{—}001885), SEQ ID NO:36 (GenBank: M20704), SEQ ID NO:38 (GenBank: BT006856), SEQ ID NO:40 (GenBank: AJ298844), SEQ ID NO:42 (GenBank: AF060222), SEQ ID NO:44 (GenBank: U10421), SEQ ID NO:46 (GenBank: NM_{—}000612), SEQ ID NO:48 (GenBank: NM_{—}033137), SEQ ID NO:50 (GenBank: AY463230), SEQ ID NO:72 (GenBank: AY339584), SEQ ID NO:74 (GenBank: AF452712), SEQ ID NO:76 (GenBank: AF002697), and SEQ ID NO:78 (GenBank: AF544398), equivalent amino acid sequence homologs (i.e., variants) within the scope of the invention also include sequences with at least 40% identity over an amino acid stretch of at least 15 consecutive residues, preferentially at least 60% identity over an amino acid stretch of at least 12 consecutive residues, and most preferred at least 80% identity over an amino acid stretch of at least 10 consecutive residues of these sequences.

For example, variants of “HtrA2/Omi” include equivalent fragments of, variants of, and fusion proteins containing SEQ ID NO:8 (GenGenBank: NM_{—}145074) and/or containing its equivalents, including those that have serine protease activity.

Exemplary variants of “Smac/Diablo” include equivalent fragments of, variants of, and fusion proteins containing SEQ ID NO:12 (GenBank: AF298770), and/or containing its equivalents, including those that inhibit IAP proteins that normally interact with caspase-9 to inhibit apoptosis.

Exemplary variants of “apoptosis-inducing factor” (AIF) include equivalent fragments of, variants of, and fusion proteins containing SEQ ID NO:10 (GenBank: AF100928), and/or containing its equivalents. AIF plays a role in redox-biochemistry and apoptosis, including as a caspase-independent death effector that produces chromatin condensation and large-scale DNA fragmentation in the cell nucleus. AIF includes flavoprotein homologous to a family of bacterial oxidoreductases, and proteins that induce morphological changes in the nucleus, including chromatin condensation and large-scale DNA fragmentation.

Exemplary variants of “cytochrome c” include equivalent fragments of, variants of, and fusion proteins containing SEQ ID NO:72 (GenBank: AY339584), and/or containing its equivalents. Cytochrome c is a component for mediating electron transfer between the primary dehydrogenases and the terminal oxidase for the oxidation of substrate with reduction of molecular oxygen to water. This electron transfer reaction is based on an oxidation-reduction of the heme iron. Cytochrome c is released from the mitochondria following the induction of apoptosis by various stimuli. Cytochrome c is part of the apoptosome which consists of Apaf-1, caspase-9, and cytochrome c. Cytochrome c release and apoptosome activation results in the activation of caspase-9 and subsequently of the effector caspase cascade

Exemplary variants of “gelsolin” include equivalent fragments of, variants of, and fusion proteins containing (SEQ ID NO:14) GenBank: BC026033), and/or containing its equivalents. Gelsolin is a multifunctional actin-binding protein obtained from mammalian cytoplasm and extracellular fluids. Plasma gelsolin differs from cellular gelsolin by the addition of 25 amino acids at the amino terminus of the molecule and both gelsolins are the product of a single gene. Plasma gelsolin has three actin-binding sites and binds with high affinity to either G-actin or F-actin. Plasma gelsolin binds a second actin molecule with a higher affinity than it binds a first actin molecule, and thus preferentially forms 2:1 complexes over 1:1 complexes and binds filaments in preference to monomers. When added to F-actin, plasma gelsolin severs the filament in a nonproteolytic manner and remains bound to one end of the newly formed filament. If free gelsolin molecules are present, they will sever the actin filament successively until only 2:1 actin-gelsolin complexes are present, thereby rapidly depolymerizing the filament. Free and complexed (to actin) gelsolin molecules differ in their functional properties. Although free gelsolin can sever actin filaments, actin-gelsol in complexes cannot. Gelsolin's primary function in the plasma is to sever actin filaments. If gelsolin is present in excess of actin, only gelsolin-actin complexes result; if actin is in excess, there are free actin oligomers and gelsolin-actin complexes. The actin severing occurs by way of a nonproteolytic cleavage of the noncovalent bond between adjacent actin molecules. Gelsolin's severing activity is activated by micromolar Ca2+ and has been shown to be inhibited by phosphatidyl inositol, bisphosphate (PIP2) and phosphatidyl inositol monophosphate (PIP). Since extracellular Ca.sup.2++ concentrations are at millimolar levels and extracellular fluids do not normally contain PIP or PIP₂ in a form that inhibits gelsolin, plasma gelsolin is constitutively active in extracellular fluids.

Exemplary variants of “caspase” include equivalent fragments of, variants of, and fusion proteins containing an enzyme that is a member of the family of enzymes that includes ICE (see H. Hara, Natl. Acad. Sci., 94, pp. 2007-2012 (1997). Caspases are central to the apoptotic program. They are cysteine protease having specificity for aspartate at the substrate cleavage site. These proteases are primarily responsible for the degradation of cellular proteins that lead to the morphological changes seen in cells undergoing apoptosis. For example, one of the caspases identified in humans was previously known as the interleukin-1-beta (IL-1-beta.) converting enzyme (ICE), a cysteine protease responsible for the processing of pro-IL-1-beta to the active cytokine. Overexpression of ICE in Rat-1 fibroblasts induces apoptosis (Miura et al., Cell 75:653 (1993)).

Other equivalents to the invention's mitochondrial malate dehydrogenase portion, minimum activator of DNA fragmentation protein, and activator of DNA fragmentation protein, included conjugates that contain these amino acid sequences, as further described below.

In a particularly preferred embodiment, the invention provides an exemplary portion of the human mitochondrial malate dehydrogenase (MDH) that comprises the minimum activator of DNA fragmentation protein is the activator of DNA fragmentation protein (“ADF”) sequence SEQ ID NO:7, is a 9 kDa C-terminal fragment of the human MDH, and which is used in Examples 2-8.

The terms “mitochondrial malate dehydrogenase” and “MDH” interchangeably refer to the exemplary amino acid sequence from human (SEQ ID NO:4) (GenGenBank: NP_{—}005909) and/or pig (SEQ ID NO:1) (GenBank: P00346), and include equivalent fragments thereof, homologs thereof, and sequences that have about the same molecular weight thereto. “Malate dehydrogenase” is an NAD(P)-dependant dehydrogenase which, in cooperation with aspartate aminotransferase isozymes, plays a pivotal role in the malate-aspartate shuttle and the pyruvate-malate shuttle. Regeneration of either mitochondrial NADH or NADPH is effected through the conversion of endogenous malate to pyruvate catalyzed by malate dehydrogenase. Four isoforms of the enzyme have been isolated from human tissue. Two human NAD(P)-dependant malate dehydrogenase isoforms have been identified; one form is present in smooth muscle and striated muscle cytoplasm, the other in the mitochondria from rapidly proliferating and tumor cells (Tanaka, T. et al. (1996) Genomics 32:128-130; Loeber, G. et al. (1991) J. Biol. Chem. 266:3016-3021). Two NAD(P)-dependant isoforms have also been identified in human breast cancer cell cytoplasm and in human hippocampal mitochondria (Chou, W. Y. (1996) J. Protein Chem. 15:272-279; Loeber, G. et al. (1994) Biochem. J. 304:687-692).

MDH is encoded by nuclear DNA. The enzyme is synthesized as a larger precursor molecule and subsequently transported into the mitochondria. An N-terminal region mediates recognition of protein targeted for this organelle and is termed the “transit peptide”. Upon binding and import to the mitochondrion, the transit peptide is removed by proteolysis and the subunits assemble to form active complexes.

Two genes encoding murine malate dehydrogenase isoforms have been identified; one is a cytosolic isoform from heart and liver and the other is a mitochondrial isoform from liver. The protein products share 23% homology. Levels of mRNA encoding the mitochondrial isoform are elevated in heart, brain, and kidney, and are relatively low in liver.

Reduced mitochondrial malate dehydrogenase activity in polymorphonuclear cells has been associated with 7-monosomy myelodysplastic syndrome, and in peripheral blood leukocytes (PBL) from Duchenne muscular dystrophy (Muchi, H. and Yamamoto, Y (1983) Blood 62:808-814; Wisniewska, W. and Lukasiuk, M. (1985) Neurol. Neurochir. Pol. 19:318-322). Significantly increased levels of mitochondrial malate dehydrogenase have been found in human breast cancer tissue, in PBL following myocardial infarction, and in PBL associated with hepatocarcinoma and acute circulatory failure (Balinsky, D. et al. (1984) J. Natl. Cancer Inst. 72:217-224; Wagenknecht, K. et al. (1988) Kardiologiia 28:55-57; Kawai, M. and Hosaki, S. (1990) Clin. Biochem. 23:327-334).

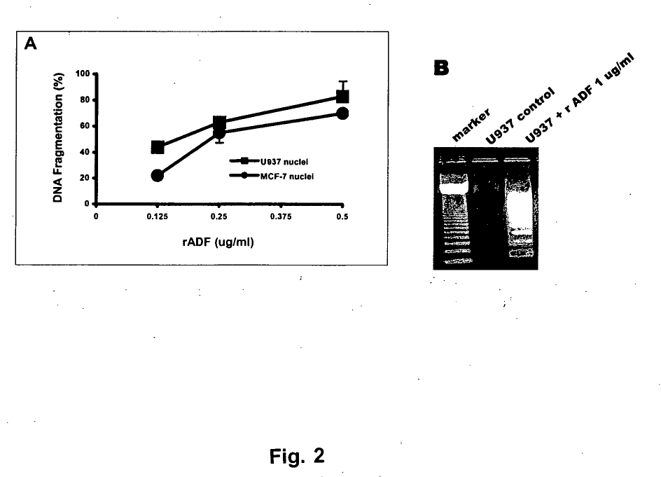

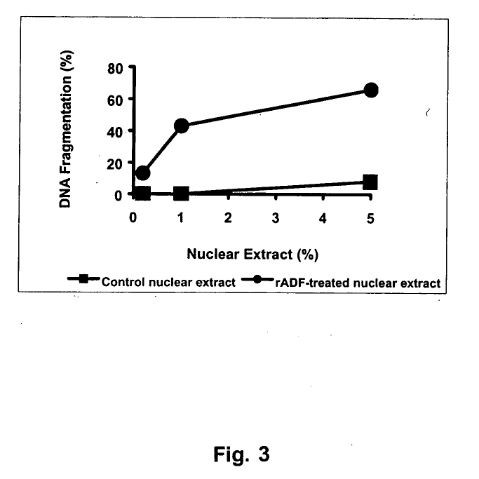

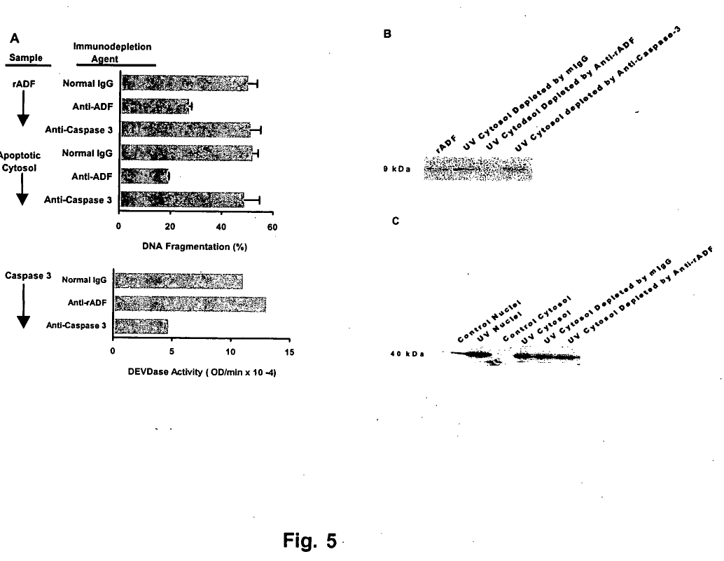

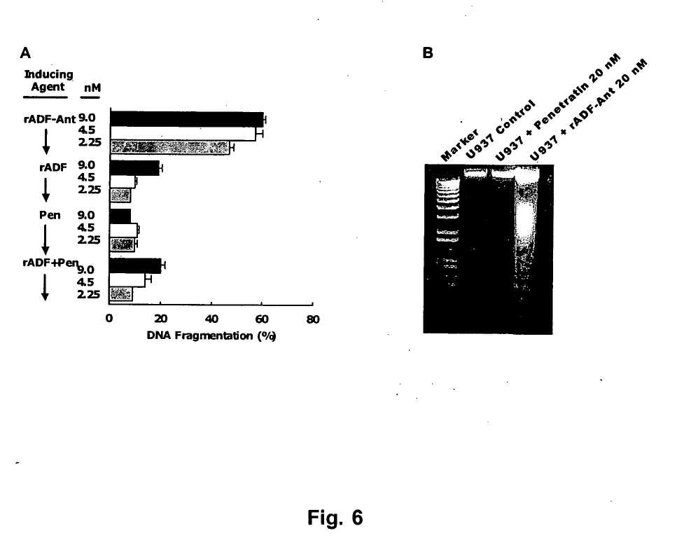

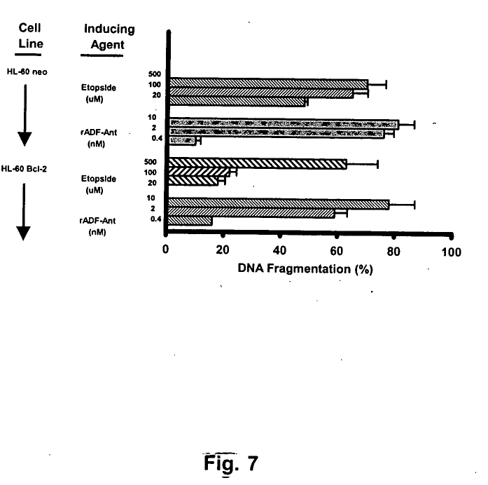

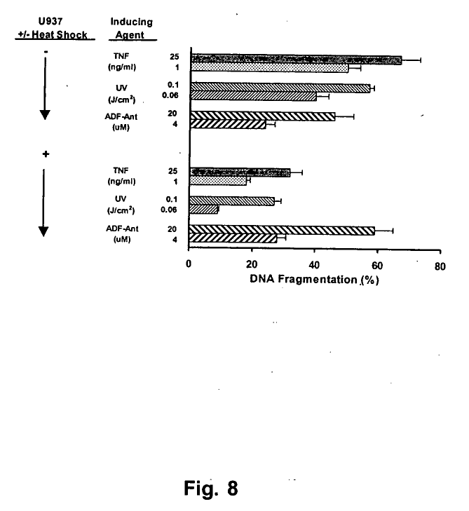

In one embodiment, the portion of MDH that comprises MADF and/or ADF has activity chosen from one or more of DNA nuclease activity and cell-killing activity. The terms “DNA nuclease activity” and “DNA fragmentation activity” are equivalent terms that refer to DNA endonuclease activity and/or DNA exonuclease activity. “DNA endonuclease activity” refers to the ability to cause (directly or indirectly, such as by activation of other proteins that have DNA nuclease activity) cleavage of phosphodiester bonds within a double- and/or single-stranded DNA. “DNA exonuclease activity” refers to the ability to cause (directly or indirectly) cleavage of successive nucleotide residues, and/or of short oliogonucleotides, from the 5′ and/or 3′ ends of double- and/or single-stranded DNA. Methods for determining DNA nuclease activity are known in the art, and are exemplified herein by measuring increased levels of release of ³H-labeled DNA fragments (Example 2, FIGS. 2 and 3; Example 4, FIG. 5A; Example 5, FIG. 6; Example 6, FIG. 7; Example, FIGS. 7 and 8). For example, data herein demonstrates that the exemplary sequence ADF (SEQ ID NO:7) has DNA nuclease activity (Examples 4 and 5).

The terms “minimum activator of DNA fragmentation” protein and “MADF” protein refer to the exemplary amino acid sequence from pig (SEQ ID NO:2) (GenBank: P00346), human (SEQ ID NO:6) (GenGenBank: NP_{—}005909), and includes equivalent fragments thereof, variants thereof, and sequences of about the same molecular weight.

The terms “activator of DNA fragmentation” protein and “ADF” protein refer to the exemplary amino acid sequence from pig (SEQ ID NO:3) (GenBank: P00346), from human (SEQ ID NO:7) (GenGenBank: NP_{—}005909), and includes equivalent fragments thereof, variants thereof, and sequences of about the same molecular weight.

The terms “Htra2/Omi,” “apoptosis-inducing factor,” “Smac/DIABLO,” “gelsolin,” “Bcl-2,” “Bax,” “Bad,”, “Bid,” “EndoG,” “caspase-activated Dnase,” “inhibitor of CAD nuclease,” “epidermal growth factor,” “vascular endothelial growth factor,” “lens crystalline protein,” “antennapedia protein,” “fibronectin type 1,” “DNase I,” “DNase II,” “human HOX protein,” “insulin-like growth factor,” “fibroblast growth factor,” “HIV Tat protein,” “Cytochrome C,” “Nix,” Nip3,” and “CIDE-B” refer to the exemplary amino acid sequences SEQ ID NOs:8, 10, 12, 14, 16, 18, 20, 22, 24, 26, 28, 30, 32, 34, 36, 38, 40, 42, 44, 46, 48, 50, 72, 74, 76, and 78, respectively, and includes equivalent fragments thereof, variants thereof, and sequences of about the same molecular weight.

Data herein shows the surprising discovery that the exemplary 9 kDa C-terminal ADF peptide fragment of MDH has cytotoxic activity (Examples 5, 6, 7, and 8), can activate Dnase(s), and induce internucleosomal DNA cleavage in isolated nuclei independently of cleavage of ICAD (Examples 4 and 5). This is in contrast to MDH, which does not have DNA nuclease and/or cell killing activity. Thus, in cells that lack CAD/DFF40, and/or caspase 3, ADF may provide an alternative pathway to activate DNA fragmentation.

Data herein demonstrates ADF's role in the apoptotic process. For example, data herein shows that ADF translocated from the mitochondria to the cytosol and nucleus in HL-60 neo cells undergoing apoptosis, but not in apoptosis-resistant HL-60 cells overexpressing Bcl-2. Second, immuno-depletion with anti-rADF of cytosols from cells committed to undergo apoptosis removed most the nuclear DNA fragmenting activity. The third piece of evidence is that introduction of rADF-Ant into whole cells rapidly and potently induced DNA fragmentation followed by cell death. This involved a modest activation of DEVDase which promoted rapid DNA fragmentation, but was not required for cell death, which still occurred at 20 h even in the presence of high concentrations of VAD-fmk.

It is surprising that a fragment of MDH (such as ADF and/or MADF) has a role in apoptosis. MDH (EC 1.1.1.37) catalyzes the reversible reduction of oxaloacetate to malate in the presence of NADH. After import of the MDH pre-protein into the mitochondria, the signal sequence is cleaved to generate mature MDH. The inventors' results showed that ADF can be detected in the mitochondria of normal cells, and that overexpression of Bcl-2 prevents release of ADF in response to UV light.

While an understanding of the mechanism of the invention is not necessary, and without intending to limit the invention to any particular mechanism, it is the inventor's opinion that ADF is not a nuclease, but rather directly or indirectly activates nucleases, such as those present in normal nuclei. Data herein indicate the putative nuclease is probably not CAD/DFF40 since ICAD/DFF45 was not cleaved in U937 nuclei treated with rADF. Other nucleases reported to reside in normal nuclei that could be the target of ADF include a c-myc-induced endonuclease, DNaseI-like enzymes including DNase1L3 and DNase γ, and endonuclease G which is found in both mitochondria and the nucleus. While mature MDH was released from mitochondria during apoptosis, however, there was no evidence reported for a role in the apoptotic process (Tafani et al. (2000) Am. J. Pathol. 156, 2111-2121). For the first time, data herein demonstrates that ADF is one of several mediators that transmit apoptotic signals to the nucleus.

Current theories of the mechanism of apoptosis propose that signals originating in the cytosol induce characteristic apoptotic nuclear changes, including chromatin condensation and DNA fragmentation into both high molecular weight and internucleosomal fragments. During apoptosis, alterations in mitochondrial function and release of mediators lead to nuclear DNA fragmentation. Apoptotic mediators released from mitochondria include cytochrome c, which in the presence of ATP forms a complex with the cytosolic Apaf-1 protein to activate caspase 9. Caspase 9, in turn, activates caspase 3. However, caspase 3 alone cannot activate DNA fragmentation in isolated nuclei, stimulating a search for downstream mediators that transmit apoptotic signals to the nucleus.

The discovery of caspase-activated DNase (CAD) (Enari et al. (1998) Proc. Natl. Acad. Sci. 95:9123-9128), referred to as DNA fragmenting factor 40 (DFF40), provided a link between the caspase cascade and induction of DNA fragmentation. It was shown that caspase 3 cleaves and inactivates an inhibitor of caspase-activated DNase (ICAD) (Enari, 1998, supra) to release active CAD. Whether this DNase is a common mediator of all forms of apoptosis is not yet known. However, the fact that many tissues do not express CAD including brain, lung, liver, skeletal muscle, thymus, testis, and small intestine, suggests to the inventors that other mediators of nuclear DNA fragmentation are likely to exist. Furthermore, DFF45 (ICAD) knockout mice are viable, show normal immune system development, and no obvious abnormalities in the major organs, suggesting that additional backup endonucleases and signaling systems may mediate developmental programmed cell death in the absence of CAD.

While an understanding of the mechanism of the invention is not necessary, and without intending to limit the invention to any particular mechanism, it is the inventor's opinion that ADF and its target nucleases may act synergistically with other enzymes such as caspases, AIF, CAD, or other endonucleases to induce rapid DNA fragmentation in cells. Since MDH is ubiquitously expressed, it is the inventor's opinion that ADF may be a common signaling molecule in apoptosis, including caspase-independent cell death.

In one embodiment, the amino acid sequences of the invention (such as those containing MDH portions, MADF, ADF, etc.) are isolated. The terms “isolated,” “to isolate,” “isolation,” “purified,” “to purify,” “purification,” and grammatical equivalents thereof as used herein, refer to the reduction in the amount of at least one contaminant (such as protein and/or nucleic acid sequence) from a sample. Thus purification results in an “enrichment,” i.e., an increase in the amount of a desirable protein and/or nucleic acid sequence in the sample. For example, the polypeptide that is to be purified may be fused to another molecule capable of binding to a ligand. The ligand may be immobilized to a solid support to facilitate isolation of the fused polypeptide. Ligand-binding systems useful for the isolation of polypeptides are commercially available and include, for example, the staphylococcal protein A and its derivative ZZ (which binds to human polyclonal IgG), histidine tails (which bind to Ni²⁺), biotin (which binds to streptavidin), maltose-binding protein (MBP) (which binds to amylose), glutathione S-transferase (which binds to glutathione), 6-8 Histidine tags in combination with Ni²⁺ chromatography, etc. It is not intended that the polypeptide probes of the present invention be limited to any particular isolation system.

In one embodiment, the amino acid sequences of the invention (such as those containing MDH portions, MADF, ADF, etc.) are recombinant. “Recombinant,” as applied to a nucleic acid sequence and/or amino acid sequence means that the sequence is the produced using molecular biology techniques (e.g., cloning, enzyme restriction and/or ligation steps).

B. Conjugates Comprising The Invention's Sequences

The invention provides conjugates that contain one or more copies of a cell-killing molecule operably linked to a second molecule. The term “conjugate” refers to two or more molecules (such as polypeptides, nucleic acid sequences, organic molecules, inorganic molecules, etc.) that are linked (directly or indirectly) together, whether by covalent or non-covalent bonds. The molecules of the conjugate may be the same or different.

The cell-killing molecule is exemplified by, but not limited to, mitochondrial proteins (such as a portion of MDH (SEQ ID NO:4) that comprises one or more of MADF (SEQ ID NO:6) and ADF (SEQ ID NO:7), Htra/Omi (SEQ ID NO:8), apoptosis inducing factor (SEQ ID NO:10), Smac/DIABLO (SEQ ID NO:12), EndoG (SEQ ID NO:24), Cytochrome C (SEQ ID NO:72), Nix (SEQ ID NO:74), Nip3 (SEQ ID NO:76), and CIDE-B (SEQ ID NO:78), etc.), and non-mitochondrial proteins (such as gelsolin (SEQ ID NO:14), Bax (SEQ ID NO:18), Bad (SEQ ID NO:20), Bid (SEQ ID NO:22), caspase-activated DNase (SEQ ID NO:26), DNase I (SEQ ID NO:40), DNase II (SEQ ID NO:42), etc.).

An advantage of the invention's cell-killing molecules is that they are potently toxic to tumor cells, including drug-resistant tumor cells, and are non-immunogenic in human subject when the molecule is derived from, or is homologous to sequences that are from, a human source.

The term “conjugate” is defined as a molecule that contains two molecules that are linked directly or indirectly to each other (e.g., via recombination or chemically) via covalent bonds or non-covalent bonds (such as those in biotin-avidin interactions, biotin-streptavidin interactions, coil-coil interactions, etc.). In one embodiment, the linked molecules have different functions (e.g., cytotoxic function, cell-binding function, etc.). In one embodiment, the conjugate molecules can be linked by an un-cleavable covalent bond, such as via a carbon-carbon bond. Alternatively, the conjugate molecules may be linked via a cleavable bond, such as a disulfide bridge cleavable by a reducing agent, an ester bond cleavable by an estearse, or peptide bond cleavable by a protease. Cleavable bonds may be desirable in order to separate the two molecules of the conjugate. The molecules of interest may be linked to the N-terminal and/or C-terminal amino acid of the amino acid sequences of the invention (such as those containing MDH portions, MADF, ADF, etc.).

In one embodiment, the conjugate's cell-killing molecule is operably linked to a second molecule. Exemplary second molecules include a cell marker recognizing molecule. Exemplary cell marker recognizing molecules include, without limitation, antibodies, enzymes, and ligands of cell receptors, as further described herein. The terms “cell marker recognizing molecule” and “cell marker recognizing compound” refer to a molecule (such as a protein, glycoprotein, etc.) that specifically binds to a cell marker molecule. The invention's conjugates that contain a cell-killing molecule and a cell marker recognizing molecule are referred to herein as “immunoconjugates.” An advantage of using cell marker recognizing molecules in conjugates with the invention's cell-killing molecules is that the cell marker recognizing molecules specifically target the cell-killing molecules of the invention to cells of interest, thereby reducing the toxicity of the cell-killing molecules to non-target cells.

The terms “cell molecule” and “cellular molecule” refer to any molecule (such as protein, carbohydrate, glycoprotein, glycolipid, nucleic acid sequence, etc.) produced by a cell, whether located in the cell, on the cell, or outside the cell (such as following cell lysis, cell death, etc.).

Cell molecules include cell marker molecules. The terms “cell marker molecule” and “marker molecule” refer to a cell molecule that is present on, and/or is produced by, a particular type of cell (such as cancer cell, epithelial cell, fibroblast cell, muscle cell, synovial cell, stem cell, embryonic cell, etc.), at a higher level than other types of cells. Cell marker molecules may be used to distinguish one type of cell from other cell types.

Exemplary cell marker molecules are illustrated by the following proteins that are further described below: PLC1, PLC2 and PLC3 (such as those produced by PLC/PRF/5 cells), sulfated glycolipids and highly acidic glycolipids such as SM3 (LacCer-II3-sulfate) and SDla (GgOse4Cer II3,IV3-disulfate) (Hiraiwa et al 1990 supra), alpha fetoprotein (AFP), membrane proteins of apparent molecular weight about 40,000 and 60,000 daltons, receptors for insulin, angiotensin II, adenosine I,-adrenalin, and rat brain nicotine and opiate receptors (Carlsson and Glad (1989) Bio/Technology 7:567-73), tyrosine kinase, G-protein coupled receptors, KM-2 glycoprotein antigen (Kumagai et al., 1992 supra), AF-20 antigen, gangliosides (such as those in PLC/PRF/5 cell), B cell receptor, CD22, CD33, renal γ-glutamyl-transferase, mucin-type glycoprotein, transferrin receptor, carcinoembryonic antigen, Lewis(y) antigen, p185^{HER2}, erbB2, E-selectin, bFGF, bFGF receptor, lutenizing hormone releasing hormone (LHRH), LHRH receptor, IL-4 receptor, IL-4, somatostatin, somatostatin receptor, endoglin, vascular endothelial growth factor (VEGF), VEGF receptor, etc.

Cell molecules include cell surface molecules and intracellular molecules. “Cell surface molecules” refers to cell molecules at least a portion of which is attached to, and/or spans, the plasma membrane. In a preferred embodiment, at least a portion of the cell surface molecule is located on the extracellular side of the plasma membrane, such that it is accessible to molecules outside the cell. For example, a polypeptide that is expressed (e.g., by the wild type cells and/or by genetic engineering) on the surface of muscle stem cells but not other cells of a cell population serves as a marker protein for the muscle stem cells. Cell surface molecules include “cell surface marker molecules”, such as an antigen to which an antibody specifically binds (e.g., in cell sorting methods to produce a population of cells enriched for cells that express the marker molecule).

Other examples of second molecules that may be linked to the invention's cell-killing molecules include, without limitation, proteins, glycoproteins, amino acid sequences, nucleotide sequences, reporter molecules such as radiolabels and fluorescent labels, chelators, cytotoxins, and carriers such as dextrans (U.S. Pat. No. 6,409,990), liposomes (Hood et. al., Science 296, 2402-2405 (2002), polyethylene glycol (Lee, et. al., Bioconjug Chem 10:973-81(1999), acrylic acid, organic drug (such as methotrexate, adriamycin chlorambucil, etc.), chemotherapeutic drug (such as 5-Fluorouracil, Leucovorin, Tomudex, Mitomycin C, CPT-11, or 3-bromopyruvate), radionuclides, enzymes, ribosome-inhibiting proteins, and toxic enzymes from plants and bacteria, as exemplified by ricin, diphtheria toxin and Pseudomonas toxin that have been coupled to antibodies or receptor binding ligands to generate cell-type-specific-killing reagents (Youle, et al., Proc. Nat'l Acad. Sci. USA77:5483 (1980); Gilliland, et al., Proc. Nat'l Acad. Sci. USA77:4539 (1980); Krolick, et al., Proc. Nat'l Acad. Sci. USA77:5419 (1980)).

Methods for using conjugates (such as immunotoxins) to cause cell death in vitro and in vivo in tumors are known in the art (Griffin, et al., IMMUNOTOXINS, p 433, Boston/Dordrecht/Lancaster, Kluwer Academic Publishers, (1988); Vitetta, et al., Science238:1098 (1987); Fitzgerald, et al., J. Nat'l Cancer Inst. 81:1455 (1989)).

Conjugates containing the invention's sequences are known such as those using chemical conjugation reactions and/or recombinant techniques. Methods for chemically linking the invention's sequences (such as MDH portions, MADF, ADF, etc.) to other molecules (such as polysaccharides, proteins, lipids, liposomes, nucleic acids, etc.) are known in the art. For example, methods for conjugating polysaccharides to peptides are exemplified by, but not limited to coupling via alpha- or epsilon-amino groups to NaIO4-activated oligosaccharide, using squaric acid diester (1,2-diethoxycyclobutene-3,4-dione) as a coupling reagent, coupling via a peptide linker wherein the polysaccharide has a reducing terminal and is free of carboxyl groups (U.S. Pat. No. 5,342,770), coupling with a synthetic peptide carrier derived from human heat shock protein hsp65 (U.S. Pat. No. 5,736,146), and using the methods of U.S. Pat. No. 4,639,512. Methods for conjugating proteins to proteins include coupling with a synthetic peptide carrier derived from human heat shock protein hsp65 (U.S. Pat. No. 5,736,146), the methods used to conjugate peptides to antibodies (U.S. Pat. Nos. 5,194,254; 4,950,480), the methods used to conjugate peptides to insulin fragments (U.S. Pat. No. 5,442,043), the methods of U.S. Pat. No. 4,639,512, and the method of conjugating the cyclic decapeptide polymyxin B antibiotic to and IgG carrier using EDAC (1-ethyl-3-(3-dimethylaminopropyl)carbodiimide)-mediated amide formation. (See e.g., Drabick et al., Antimicrob. Agents Chemother., 42:583-588 (1998)). Approaches to conjugate nucleic acids to proteins are also known in the art, such as those described in U.S. Pat. Nos. 5,574,142; 6,117,631; and 6,110,687; each of is incorporated in its entirety by reference. Methods for conjugating lipids to peptides have been described in the art including, but not limited to, the use of reductive amination and an ether linkage which contains a secondary or tertiary amine (U.S. Pat. No. 6,071,532), the methods of U.S. Pat. No. 4,639,512, the methods used for covalently coupling peptides to unilamellar liposomes (Friede et al., Vaccine, 12:791-797 (1994)), of coupling human serum albumin to liposomes using the hetero-bifunctional reagent N-succinimidyl-S-acetylthioacetate (SATA) (Kamps et al., Biochim. Biophys. Acta, 1278:183-190 (1996)), of coupling antibody Fab′ fragments to liposomes using a phospholipid-poly(ethylene glycol)-maleimide anchor (Shahinian et al., Biochim. Biophys. Acta, 1239:157-167 (1995)), and of coupling Plasmodium CTL epitope to palmitic acid via cysteine-serine spacer amino acids (Verheul et al., J. Immunol. Methods, 182:219-226 (1995)). Alternatively, conjugates of two proteins may be generated by recombinant techniques to form a fusion protein.

Exemplary conjugates are further described below under 1. Conjugates Containing N-Terminal Signal Peptides, 2. Conjugates Containing Cell Internalization Peptides, 3. Conjugates Containing Nuclear Localization Peptides, 4. Conjugates Containing Nuclides, 5. Conjugates Containing Biotin Binding Proteins, 6. Conjugates Containing Proteins, and 7. Conjugates Containing Antibodies.

1. Conjugates Containing N-Terminal Signal Peptides

In one embodiment, conjugates that contain the invention's sequences (such as MDH portions, MADF, ADF, etc.) and that are within the scope of the invention contain one or more N-terminal signal peptides. The terms “N-terminal signal peptide,” “N-terminal signal sequence,” “signal peptide” and “signal sequence” refer to a sequence of amino acids that is usually (but not necessarily) located within the N-terminal portion of a protein, and that facilitates the secretion of the protein outside the cell. The signal sequence may be cleaved during the secretion process, thus resulting in a protein that lacks the signal sequence. Secretion signals suitable for use are widely available and are well known to one skilled in the art (von Heijne, J. Mol. Biol. 184: 99-105, 1985). Signal sequences are exemplified, but not limited to, prokaryotic and eukaryotic secretion signals that are functional in E. coli (or other host), including, but not limited to, those encoded by the following E. coli genes: pelB (Lei et al., J. Bacteriol. 169: 4379, 1987), phoA, ompA, ompT, ompF, ompC, beta-lactamase, and alkaline phosphatase.

2. Conjugates Containing Cell Internalization Peptides

In another embodiment, conjugates that contain the invention's proteins (such as MDH portions, MADF, ADF, etc.) and that are within the scope of the invention contain one or more cell internalization peptides. The inclusion of one or more internalization sequences in the invention's fusion proteins is advantageous where it is desired to bring about the DNA nuclease activity and/or cell killing activity of the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase H). For example, internalization allows the conjugate to exert its effect while reducing side effects that may be associated with the conjugate's presence on the outside of the cell membrane. Additionally, internalization of the invention's proteins is advantageous in applications where toxic side reactions (such as following administration to an animal) are preferably reduced.

The term “cell internalization peptide” refers to a peptide that increases the translocation of a protein that is linked to the internalization peptide across the cytoplasmic membrane into the cell cytoplasm. Internalization of fusion proteins that contain a cell internalization peptide may be determined using methods know in the art such those described herein (Example 5), including detecting the presence of the fusion protein and/or its activity in the cytoplasm. Exemplary cell internalization peptides include Penetratin (SEQ ID NO:57) Arg-Gln-Ile-Lys-Ile-Trp-Phe-Gln-Asn-Arg-Arg-Met-Lys-Trp-Lys-Lys. Penetratin is a 16 amino acid peptide derived from the Drosophila homeodomain transcription factor antennapedia (reviewed in Derossi et al., 1998, Trends Cell Biol 8:84-87). Data herein demonstrates that Penetratin efficiently translocates rADF in the fusion protein (rADF-Ant) into normal cells (Example 5) and tumor cells (Examples 6 and 7). Other examples of cell internalization peptides include sequences containing from 6 to 9 arginine residues, containing the HIV-1 Tat (amino acids 37-72) (SEQ ID NO:58): CFITKALGISYGRKKRRQRRRPPQGSQTHQVSLSKQ (Fawell et al. 1994 Proc Natl Acad Sci 91:664-668); Truncated Tat (amino acids 48-60) (SEQ ID NO:59): GRKKRRQRRRPPQC (Vives et al. 1997 J Biol Chem 272:16010-16017); HIV-1 Rev (amino acids 34-50) (SEQ ID NO:60): TRQARRNRRWRERQR (Suzuki et al. 2002 J Biol Chem 277:2437-2443); HSV-1 structural protein VP22 (amino acids #1-367 (Elliot et al. 1997 Cell 88:223-233); and Pep-1 (SEQ ID NO:61): KETWWETWWTEWSQPKKKRKV (Morris et al. 2001 Nature Biotech 19:1173-1176). Unlike the other above peptides, Pep-1 can transport cargoes across cell membranes without being physically linked to the cargo.

3. Conjugates Containing Nuclear Localization Peptides

In a further embodiment, conjugates that contain the invention's proteins (such as MDH portions, MADF, ADF, etc.) and that are within the scope of the invention contain one or more nuclear localization peptides. The term “nuclear localization peptide” refers to a peptide that increases the translocation of a protein that is linked to the internalization peptide across the nuclear membrane into the nucleus. Internalization of fusion proteins that contain a nuclear localization peptide may be determined using methods know in the art, including detecting the presence of the fusion protein and/or its activity in the nucleus. Exemplary cell internalization peptides include (SEQ ID NO:62) PKKKRKV from SV40T; c-Myc derived sequence (SEQ ID NO:63): PAAKRVKLD (Dang et al. 1988 Mol Cell Biol. 8:4048-4054); HIV-1 Tat derived sequence (SEQ ID NO:64): GRKKRRQRRRAP (Dang et al. 1989 J Biol Chem 264:18019-18023); c-Myb derived sequence (SEQ ID NO:65): PLLKKIKQ (Dang et al. 1989 supra); N-myc derived sequence (SEQ ID NO:66): PPQKKIKS (Dang et al. 1989 supra); P53 derived sequence (SEQ ID NO:67): PQPKKKP (Dang et al. 1989 supra); c-erb-A derived sequence (SEQ ID NO:68): SKRVAKRKL (Dang et al. 1989 supra); and Lactoferrin derived sequence (SEQ ID NO:69): GRRRR (Penco et al. 2001 Biotechnol Appl Biochem 34:151-159).

4. Conjugates Containing Nuclides

In a further embodiment, conjugates that contain the invention's proteins (such as MDH portions, MADF, ADF, etc.) and that are within the scope of the invention contain one or more “radionuclide,” such as, such as Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, 152, Gadolinium-153, Gold-195, Gold-199, Hainium-175, Hafnium-175-181, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-226, Rhemum-186, Rubidium-86, Ruthenium-103, Ruthenium-106, Scandium-44, Scandium46, Selenium-75, Silver-110 m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Titanium-44, Tungsten-185, Vanadium-48, Vanadium-49, Ytterbium-169, Yttrium-88, Yttrium-90, Yttrium-91, Zinc-65, and Zirconium. In a preferred embodiment, the nuclide is conjugated to cell molecules that are themselves conjugated to the invention's cell-killing molecules.

5. Conjugates Containing Biotin Binding Proteins

In yet another embodiment, conjugates that contain the invention's proteins (such as MDH portions, MADF, ADF, etc.) and that are within the scope of the invention contain one or more biotin-binding protein (such as an antibody that is specific for biotin).

6. Conjugates Containing Proteins

In one embodiment, the invention's conjugates include a fusion protein containing one or more cell-killing molecule. In one embodiment, the cell-killing molecule is a mitochondrial protein, as exemplified by a portion of MDH (SEQ ID NO:4) that comprises one or more of MADF (SEQ ID NO:6) and ADF (SEQ ID NO:7), Htra/Omi (SEQ ID NO:8), apoptosis inducing factor (SEQ ID NO:10), Smac/DIABLO (SEQ ID NO:12), EndoG (SEQ ID NO:24), Cytochrome C (SEQ ID NO:72), Nix (SEQ ID NO:74), Nip3 (SEQ ID NO:76), and CIDE-B (SEQ ID NO:78). In another embodiment, the cell-killing molecule is a non-mitochondrial protein as exemplified by gelsolin (SEQ ID NO:14), Bax (SEQ ID NO:18), Bad (SEQ ID NO:20), Bid (SEQ ID NO:22), caspase-activated DNase (SEQ ID NO:26), DNase I (SEQ ID NO:40), and DNase II (SEQ ID NO:42).

The term “fusion protein” refers to two or more polypeptides that are operably linked. The term “operably linked” when in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest. Also, DNA encoding a secretory leader is operably linked to DNA encoding a polypeptide of interest if it is expressed as a polypeptide that participates in the secretion of the polypeptide of interest; a promoter or enhancer is operably linked to a coding sequence if it brings about the transcription of the sequence; a ribosome binding site is operably linked to a coding sequence if it is positioned so as to increase translation. Generally, alghough not necessarily, “operably linked” means that the DNA sequences being linked are contiguous, and, in the case of a secretory leader, contiguous and in reading phase. However, enhancers and other sequences need not be contiguous. Linking is accomplished by ligation at convenient restriction sites. If such sites do not exist, the synthetic oligonucleotide adaptors or linkers are used in accordance with conventional practice. The term also refers to the linkage of amino acid sequences in such a manner so that a functional protein is produced.

For example, the conjugates of the invention contain one or more copies of one or more of a mitochondrial malate dehydrogenase (MDH) portion, minimum activator of DNA fragmentation protein (MADF), and activator of DNA fragmentation protein (ADF). Data herein demonstrates that recombinant ADF genetically fused to penetratin (a cell internalization peptide) and expressed as the fusion protein rADF-Ant induced internucleosomal DNA fragmentation in isolated tumor cell nuclei. Data herein shows that when genetically fused to cell-penetrating peptides such as Ant or bFGF, ADF potently killed a variety of tumor cell types (Example 6). Since data herein demonstrates that linking ADF to another protein does not destroy its activity, it is the inventors' opinion that fusing rADF to an scFv will not reduce rADF's cell killing and/or nuclease activity. Data herein also shows that in tests of 7 different model systems of chemotherapeutic drug resistant cells, each of the selected cell variants were still completely sensitive to killing by the exemplary rADF-Ant (Examples 7-8). Even the intrinsically drug-resistant hepatocellular carcinoma (“HCC”) lines that were rendered more highly resistant by adherence to FN were still susceptible to rADF-Ant. Therefore, this remarkably potent human peptide is an ideal warhead to arm the anti-HCC scFv described herein.

In another embodiment, the conjugates of the invention may contain one or more Bcl-2 family members such as Bax, Bad, and Bid. The term “BCL-2” includes equivalent fragments and variants of SEQ ID NO:16 (GenBank: M14745). BCL-2 is a human proto-oncogene located on chromosome 18. Its product is an integral membrane protein (called Bcl-2) located in the membranes of the endoplasmic reticulum (ER), nuclear envelope, and in the outer membranes of the mitochondria. The gene was discovered as the translocated locus in a B-cell leukemia (hence the name). Bcl-2 itself is an anti-apoptotic protein, which is activated by chromosomal translocations in non-Hodgkin lymphomas and is also inappropriately overexpressed in many solid tumors, contributing to resistance to chemotherapy and radiation-induced apoptosis. Unlike many other known human oncogenes, Bcl-2 exerts its influence by enhancing cell survival rather than stimulating cell division. Bcl-2 proteins regulate apoptosis and function to either inhibit or promote cell death. Over expression of members such as Bcl-2 and Bcl-xL inhibit the apoptotic process (Huang Z. 2000. Bcl-2 family proteins as targets for anticancer drug design. Oncogene 19(56): 6627-6631; Reed JC. 1997. Double identity for proteins of the Bcl-2 family. Nature 387(6635): 773-776). Bcl-2 family members are also characterized by dimerizing to further modulate apoptosis.

Bax and Bak have been shown to play a critical role in cytochrome c release from mitochondria and thus initiate apoptosis (Wei et al., 2001. Proapoptotic BAX and BAK: a requisite gateway to mitochondrial dysfunction and death. Science 292(5517): 624-626).

In another embodiment, the conjugates of the invention contain one or more copies of Bad. The term “Bad” includes equivalent fragments and variants of SEQ ID NO:20 (GenBank:AF031523). Bad plays a critical role in the Bax-mediated apoptosis pathway by dimerizing with Bcl-xL, causing the displacement of Bax. The displacement of Bax allows apoptosis to proceed (Yang et al., 1995. Bad, a heterodimeric partner for Bcl-XL and Bcl-2, displaces Bax and promotes cell death. Cell 80(2): 285-291). Bcl-xS, a shorter version of Bcl-xL (lacking amino acids 126-188), apparently utilizes a different pathway than Bax to induce cell death. Some research suggests that Bcl-xS uses a novel mechanism for regulating caspase or it may use an alternate cell death effector pathway (Fridman et al. 2001. Cytochrome c depletion upon expression of Bcl-XS. J Biol Chem 276(6): 4205-10; Lindenboim L, Yuan J, Stein R. 2000. Bcl-xS and Bax induce different apoptotic pathways in PC12 cells. Oncogen 19(14): 1783-1793).

In a further embodiment, the conjugates of the invention contain one or more copies of BID. The term “BID” includes equivalent fragments and variants of SEQ ID NO:22 (GenBank: AF250233). BID was recently identified as a factor that provides a link between Fas receptor activated Caspase-8 and the release of cytochrome c from the mitochondria (Luo et al., 1998, Cell, 94: 481-490 & Li et al.,1998, Cell, 94: 491-501). This factor is BID, a member of the BH3 subfamily known to interact with Bcl-2 and Bax through its BH3 domain (Wang et al., 1996, Genes Dev., 10: 2859-2869). BID (26 kDa) is cleaved by Caspase-8 into fragments of 15 kDa (C-terminus) and 11 kDa (N-terminus). The 15 kDa fragment tBID (=truncated BID) contains the BH3 domain and, indeed, is the functional part of BID for cytochrome c release. It was shown that after Caspase-8 cleavage, the C-terminal BID fragment (tBID) translocates to the mitochondria. In a time course experiment after Fas stimulation of living cells (Jurkat) the sequential activation of Caspase-8, BID, Caspase-3 and DFF was shown.

In another embodiment, the conjugates of the invention contain one or more copies of Bax. The term “Bax” includes equivalent fragments and variants of SEQ ID NO:18 (GenBank: NM_{—}138764). The solution structure of BID, determined by NMR, suggests two modes of proapoptotic action: (1) BID can interact by its BH3 domain with the anti-apoptotic Bcl-XL and thus prevent the formation of the antiapoptotic complex between Bcl-XL and Apafl. Truncation of BID by caspase-8 is supposed to enhance the heterodimerization with Bcl-XL; (2) BID contains the structural motifs for pore-formation, and after truncation it is potentially able to form selective ion-channels similar to BAX and may promote apoptosis in a way other than inhibiting Bcl-2 proteins and independent from its BH3 domain (Chou et al., 1999, Cell, 96: 615-624; McDonnell et al., 1999, Cell, 96: 625-634).

The conjugates of the invention may contain one or more intracellular nucleases such as EndoG, DNase I, DNase II, caspase-activated DNase (CAD). A conjugate also might contain a fragments of CAD that is more resistant than full-length CAD to inhibition by ICAD.

The term “EndoG” includes equivalent fragments and variants of SEQ ID NO:24 (GenBank: NM_{—}004435). EndoG is a mitochondrial protein that participates in apoptosis. EndoG is encoded by a nuclear gene, translated in the cytosol, and subsequently imported into the mitochondria. It has been proposed to participate in mitochondrial replication by forming RNA primers for the initiation of mitochondrial DNA synthesis. The proposed function was based on its location and substrate specificity, since EndoG prefers GC-rich DNA substrates, which resemble DNA sequence in mitochondrial DNA replication origin. Release of EndoG from apoptotic mitochondria occurs at a rate similar to that of cytochrome c, suggesting that it is also in the mitochondrial intermembrane space. Once released, EndoG is able to induce nucleosomal DNA fragmentation. Unlike DFF/CAD (DNA fragmentation factor/caspase-activated deoxyribonuclease), the apoptotic nucleases that require caspase-3 cleavage of the DFF45/ICAD (45-kDa subunit of DFF/inhibitor of CAD) to activate, EndoG activity is independent of caspase activation. Furthermore, EndoG activity may be responsible for DNA fragmentation observed in DFF45-deficient mouse embryonic fibroblast cells after induction of apoptosis by treatment with ultraviolet light and tumor necrosis factor.

The term “Deoxyribonuclease I” includes equivalent fragments and variants of SEQ ID NO:40 (GenBank: AJ298844). Deoxyribonuclease I is an endonuclease, splitting phosphodiester linkages, preferentially adjacent to a pyrimidine nucleotide yielding 5′-phosphate terminated polynucleotides with a free hydroxyl group on position 3′. The average chain of limit digest is a tetranucleotide. DNase I acts upon single chain DNA, and upon double-stranded DNA and chromatin. In the latter case, although histones restrict susceptibility to nuclease action, over a period of time nearly all chromatin DNA is acted upon.

The term “Deoxyribonuclease II” (DNase II) includes equivalent fragments and variants of SEQ ID NO:42 (GenBank: AF060222). DNase II is a lysosomal DNase that has been implicated in the degradation of DNA in apoptotic cells.

The term “caspase activated DNase” (CAD) includes equivalent fragments and variants of SEQ ID NO:26 (GenBank: AB013918). CAD causes the fragmentation of DNA into nucleosomal units, as seen in DNA laddering assays. Normally CAD exists as an inactive complex with ICAD (inhibitor of CAD, also known as DNA fragmentation factor45). During apoptosis, ICAD is cleaved by caspases, including caspase 3, to release CAD. Since CAD is a DNase with a high specific activity (comparable to or higher than DNase I and DNase II) rapid fragmentation of the nuclear DNA follows.

7. Conjugates Containing Antibodies

Exemplary fusion partners with the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase H) include an antibody. The terms “antibody” and “immunoglobulin” are interchangeably used to refer to a glycoprotein or a portion thereof (including single chain antibodies), which is evoked in an animal by an immunogen and which demonstrates specificity to the immunogen, or, more specifically, to one or more epitopes contained in the immunogen. The term “antibody” includes polyclonal antibodies, monoclonal antibodies, naturally occurring antibodies as well as non-naturally occurring antibodies, including, for example, single chain, chimeric, bifunctional, de-immunized, and humanized antibodies, as well as antigen-binding fragments thereof, including, for example, Fab, F(ab′)₂, Fab fragments, Fd fragments, and Ev fragments of an antibody, as well as a Fab expression library.

It is intended that the term “antibody” encompass any immunoglobulin (e.g., IgG, IgM, IgA, IgE, IgD, etc.) obtained from any source (e.g., humans, rodents, non-human primates, caprines, bovines, equines, ovines, etc.). Genes encoding antibodies include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Several different regions of an antibody contain conserved sequences. Extensive amino acid and nucleic acid sequence data displaying exemplary conserved sequences is compiled for immunoglobulin molecules by Kabat et al., in Sequences of Proteins of Immunological Interest, National Institutes of Health, Bethesda, Md., 1987.

In one embodiment, an antibody comprises a tetramer. A tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. In one embodiment, the variable region of the heavy or light chain comprises four framework regions each containing relatively lower degrees of variablity that includes lengths of conserved sequences. Framework regions are typically conserved across several or all immunoglobulin types and thus conserved sequences contained therein are particularly suited for preparing repertoires having several immunoglobulin types.

The term “polyclonal antibody” refers to an immunoglobulin produced from more than a single clone of plasma cells; in contrast “monoclonal antibody” refers to an immunoglobulin produced from a single clone of plasma cells. Monoclonal and polyclonal antibodies may or may not be purified. For example, polyclonal antibodies contained in crude antiserum may be used in this unpurified state.

Those skilled in the art know how to make polyclonal and monoclonal antibodies which are specific to a desirable polypeptide. For the production of monoclonal and polyclonal antibodies, various host animals can be immunized by injection with the peptide corresponding to any molecule of interest in the present invention, including but not limited to rabbits, mice, rats, sheep, goats, etc. For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein (Kohler and Milstein, Nature, 256:495-497 (1975)), techniques using germ-free animals and utilizing technology such as that described in PCT/US90/02545, as well as the trioma technique, the human B-cell hybridoma technique (See e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)). In some particularly preferred embodiments of the present invention, the present invention provides monoclonal antibodies of the IgG class.

The invention also contemplates humanized antibodies. A “humanized antibody” is an antibody in which the antigen-recognition sites (CDRs) or complementarily-determining hypervariable regions are of non-human origin, and framework regions (FR) of variable domains are of human origin. Humanized antibodies are preferred over non-human antibodies when in certain applications, such as when used in human subjects. Humanized antibodies may be generated using methods known in the art, including those described in U.S. Pat. Nos. 5,545,806, 5,569,825 and 5,625,126, the entire contents of which are incorporated by reference.

In one method of humanization of an animal monoclonal anti-hepatoma antibody, RPAS is combined with the CDR grafting method described by Daugherty et al., Nucl. Acids Res., 19:2471-2476, 1991. Briefly, the variable region DNA of a selected animal recombinant anti-hepatoma ScFv is sequenced by the method of Clackson, T., et al., Nature, 352:624-688, 1991, incorporated herein by reference. Using this sequence, animal CDRs are distinguished from animal framework regions (FR) based on locations of the CDRs in known sequences of animal variable genes. Kabat, H. A., et al., Sequences of Proteins of Immunological Interest, 4th Ed. (U.S. Dept. Health and Human Services, Bethesda, Md., 1987). Once the animal CDRs and FR are identified, the CDRs are grafted onto human heavy chain variable region framework by the use of synthetic oligonucleotides and polymerase chain reaction (PCR) recombination. Codons for the animal heavy chain CDRs, as well as the available human heavy chain variable region framework, are built in four (each 100 bases long) oligonucleotides. Using PCR, a grafted DNA sequence of 400 bases is formed that encodes for the recombinant animal CDR/human heavy chain FR protection.

In order to retain the antigen-binding properties of the original antibody, the structure of its combining-site is faithfully reproduced in the humanized version. This can be achieved by transplanting the combining site of the nonhuman antibody onto a human framework, either (a) by grafting only the nonhuman CDRs onto human framework and constant regions with or without retention of critical framework residues (Jones et al., Nature, 321:522 (1986); Verhoeyen et al., Science, 239:1539 (1988)); or (b) by transplanting the entire nonhuman variable domains (to preserve ligand-binding properties) but also “cloaking” them with a human-like surface through judicious replacement of exposed residues (to reduce antigenicity) (Padlan, Molec. Immunol., 28:489 (1991)).

One method of identifying the framework residues which need to be preserved is by computer modeling. Alternatively, critical framework residues may potentially be identified by comparing known antibody combining site structures (Padlan, Molec. Immun., 31(3):169-217 (1994)).

The residues which potentially affect antigen binding fall into several groups. The first group comprises residues that are contiguous with the combining site surface and which could therefore make direct contact with antigens. They include the amino-terminal residues and those adjacent to the CDRs. The second group includes residues that could alter the structure or relative alignment of the CDRs either by contacting the CDRs or the opposite chains. The third group comprises amino acids with buried side chains that could influence the structural integrity of the variable domains. The residues in these groups are usually found in the same positions (ibid.) according to the adopted numbering system. See Kabat et al., Sequences of Proteins of Immunological Interest, NIH Pub. No. 91-3242 (5th ed., 1991) (U.S. Dept. Health & Human Services, Bethesda, Md.) and Genbank.

To form the humanized variable region, amino acids in the human acceptor sequence may be replaced by the corresponding amino acids from the donor sequence if they are in one of the following categories: (1) the amino acid is in a CDR. Additional amino acids in the acceptor immunoglobulin chain may be replaced with amino acids from the CDR-donor immunoglobulin chain. More specifically, further optional substitutions of a human framework amino acid of the acceptor immunoglobulin with the corresponding amino acid from a donor immunoglobulin will be made at positions which fall in one or more of the following categories: (2) the amino acid in the human framework region of the acceptor immunoglobulin is rare for that position and the corresponding amino acid in the donor immunoglobulin is common for that position in human immunoglobulin sequences; or (3) the amino acid is immediately adjacent to one of the CDR'S; or (4) the amino acid is predicted to be within about 3 angstroms of the CDR's in a three-dimensional immunoglobulin model and capable of interacting with the antigen or with the CDR's of the donor or humanized immunoglobulin. Moreover, an amino acid in the acceptor sequence may optionally be replaced with an amino acid typical for human sequences at that position if (5) the amino acid in the acceptor immunoglobulin is rare for that position and the corresponding amino acid in the donor immunoglobulin is also rare, relative to other human sequences.

The humanized immunoglobulin chain typically comprises at least about 3 amino acids from the donor immunoglobulin in addition to the CDR's, usually at least one of which is immediately adjacent to a CDR in the donor immunoglobulin. The heavy and light chains may each be designed by using any one or all three of the position criteria.

When combined into an intact antibody, the humanized light and heavy chains of the present invention are substantially non-immunogenic in humans and retain substantially the same affinity as the donor immunoglobulin to the antigen (such as a protein or other compound containing an epitope). These affinity levels may be within about 4 fold, preferably within about 2 fold of the donor immunoglobulin. More preferably, the humanized antibodies will exhibit affinity levels at least about 60% to 90% of the donor immunoglobulin's original affinity to the antigen.

The expression of recombinant CDR-grafted immunoglobulin gene is accomplished by its transfection into human 293 cells (transformed primary embryonic kidney cells, commercially available from American Type Culture Collection, Rockville, Md. 20852) which secrete fully grafted antibody. See, e.g., Daugherty, B. L., et al., Nucl. Acids Res., 19:2471-2476 (1991).

The invention contemplates an antibody fragment. The term “antibody fragment” refers to a portion of the antibody. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, and Fc fragments. The antibody fragment can optionally be a single chain antibody fragment. Alternatively, the fragment can comprise multiple chains which are linked together, for instance, by disulfide linkages. The fragment can also optionally be a multimolecular complex. In a preferred embodiment, the antibody fragment comprises at least about 50 amino acids and more preferably at least about 200 amino acids.

Techniques described for the production of single chain antibody fragments (“scFv”) (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 (1989)) to allow rapid and easy identification of monoclonal Fab fragments with the desired specificity. For instance, hybridoma cells secreting selected protective anti-hepatoma antibodies are used in the production of recombinant anti-hepatoma antibodies. For example, Pharmacia's (Pharmacia LKB Biotechnology, Sweden) “Recombinant Phage Antibody System” (RPAS) may be used for this purpose. In the RPAS, antibody variable heavy and light chain genes are separately amplified from the hybridoma mRNA and cloned into an expression vector. The heavy and light chain domains are co-expressed on the same polypeptide chain after joining with a short linker DNA which codes for a flexible peptide. This assembly generates a single-chain Fv fragment (ScFv) which incorporates the complete antigen-binding domain of the antibody. Using the antigen-driven screening system, the ScFv with binding characteristics equivalent to those of the original monoclonal antibody is selected (See, e.g., McCafferty, J., et al., Nature, 348:552-554, 1990; Clackson, T., et al., Nature, 352:624-688, 1991). The recombinant ScFv includes a considerably smaller number of epitopes than the intact monoclonal antibody, and thereby represents a much weaker immunogenic stimulus when injected into humans. An intravenous injection of ScFv into humans is, therefore, expected to be more efficient and immunologically tolerable in comparison with currently used whole monoclonal antibodies (Norman, D. J., et al., Transplant Proc., 25, suppl. 1:89-93, 1993).

The invention contemplates antibody fragments that contain the idiotype (antigen binding region) of the antibody molecule. Such fragments can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent. Genes encoding antigen binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

The invention contemplates chimeric antibodies. As used herein, “chimeric antibodies” contain portions of two different antibodies, typically of two different species. Generally, such antibodies contain human constant regions and variable regions of another species, typically murine variable regions. For example, some mouse/human chimeric antibodies have been reported which exhibit binding characteristics of the parental mouse antibody, and effector functions associated with the human constant region. See, e.g.: U.S. Pat. No. 4,816,567 to Cabilly et al.; U.S. Pat. No. 4,978,745 to Shoemaker et al.; U.S. Pat. No. 4,975,369 to Beavers et al.; and U.S. Pat. No. 4,816,397 to Boss et al. Generally, these chimeric antibodies are constructed by preparing a genomic gene library from DNA extracted from pre-existing murine hybridomas (Nishimura et al., Cancer Res., 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes are then expressed in a cell line of choice, usually a murine myeloma line. Such chimeric antibodies have been used in human therapy.

The invention also contemplates “de-immunized” antibodies, i.e., antibodies whose sequence has been modified to reduce, and more preferably eliminate, T-cell binding to the modified sequence. The de-immunization technology was invented by Frank Carr (European patent AU1667600). The peptide-MHC class II complexes can be recognized by T cells and can trigger the activation and differentiation of helper T cells. Helper T cells are required to initiate and sustain immunogenicity through interaction with B cells, resulting in secretion of antibodies that bind specifically to the administered biologic molecule (such as antibody).

For de-immunization of biologic molecules (such as antibody), helper T cell epitopes are identified within the sequence of the biologic molecule and these sequences are modified, principally by amino acid substitution, to avoid recognition by T cells. As a result of de-immunization, the biologic molecule can no longer trigger T cell help and the subsequent production of antibodies directed against the biologic. In this way, the modified biologic molecule can circumvent the immunogenicity that is frequently associated with biologic molecules containing T cell epitopes.

From these alternatives, individual substitutions are selected which are considered to have the lowest risk of compromising the therapeutic actions of the antibody or protein biologic molecule. These substitutions are then tested within the whole biologic molecule to measure activity of the altered biologic molecule. Substitutions required to remove individual T cell epitopes are then combined within the biologic molecule to produce a modified form of the biologic molecule that is unable to activate helper T cells.

Even when helper T cell epitopes coincide with important functional sites within the biologic molecule, there is usually scope for amino acid substitutions to remove the T cell epitopes without appreciable loss of activity and, in the case of antibodies, there are several cases of increased binding activity as a result of de-immunization. Where such substitutions are difficult to identify, other de-immunization strategies are employed such as substitutions that maintain MHC binding but evade T cell recognition, or substitutions that facilitate protease cleavage of the T cell epitope sequence.

To de-immunize VH and VL sequences of antibodies, these sequences are analyzed using the human T cell epitope identification toolbox. The result is a human T cell epitope “map” from each V region showing the location of epitopes in relation to complementarily-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are “designed” comprising combinations of amino acid substitutions and these sequences are subsequently incorporated into a range of modified antibodies that are tested for function. For a typical antibody de-immunization project, between 12 and 24 variant antibodies are generated and tested.

VH and VL genes from the starting antibody are subjected to one or more rounds of mutation using synthetic oligonucleotide primers. The resultant VH and VL genes are then cloned into plasmid vectors adjacent to heavy and light chain constant (C) region genes (CH and CL respectively). Depending on the required properties of the final therapeutic antibody, natural human IgG1 or IgG4 C region genes are used or, alternatively, human constant regions modified to alter antibody effector functions such as Fc receptor binding.

Complete heavy and light chain genes comprising modified V and human C regions are then cloned into mammalian expression vectors and the subsequent plasmids introduced into rodent myeloma cell lines for the production of whole antibody. The alternative antibodies (comprising different modifications within their VH and VL sequences) are compared in appropriate biochemical and biological assays, and the optimal variant is identified.

In the production of antibodies, screening for the desired antibody can be accomplished by techniques known in the art (e.g., radioimmunoassay, ELISA (enzyme-linked immunosorbent assay), “sandwich” immunoassays, immunoradiometric assays, gel diffusion precipitin reactions, immunodiffusion assays, in situ immunoassays (e.g., using colloidal gold, enzyme or radioisotope labels), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay.

Antibodies specifically bind to an antigenic determinant. The terms “antigenic determinant” and “epitope” as used herein refer to that portion of an antigen that makes contact with a particular antibody variable region. When a protein or fragment of a protein is used to immunize a host animal, numerous regions of the protein may induce the production of antibodies which bind specifically to a given region or three-dimensional structure on the protein; these regions or structures are referred to as antigenic determinants.

The terms “specific binding,” “binding specificity,” and grammatical equivalents thereof when made in reference to the binding of a first molecule (such as a polypeptide, glycoprotein, nucleic acid sequence, etc.) to a second molecule (such as a polypeptide, glycoprotein, nucleic acid sequence, etc.) refer to the preferential interaction between the first molecule with the second molecule as compared to the interaction between the second molecule with a third molecule. Specific binding is a relative term that does not require absolute specificity of binding; in other words, the term “specific binding” does not require that the second molecule interact with the first molecule in the absence of an interaction between the second molecule and the third molecule. Rather, it is sufficient that the level of interaction between the first molecule and the second molecule is higher than the level of interaction between the second molecule with the third molecule. “Specific binding” of a first molecule with a second molecule also means that the interaction between the first molecule and the second molecule is dependent upon the presence of a particular structure on or within the first molecule; in other words the second molecule is recognizing and binding to a specific structure on or within the first molecule rather than to nucleic acids or to molecules in general. For example, if a second molecule is specific for structure “A” that is on or within a first molecule, the presence of a third nucleic acid sequence containing structure A will reduce the amount of the second molecule which is bound to the first molecule.

The term “binding affinity” as used herein in reference to the binding of an antibody to another molecule refers to the level of binding of the antibody to the molecule. In one embodiment, the invention utilizes antibodies whose binding affinity to SEQ ID NO:7 and/or SEQ ID NO:6 is higher than the binding affinity of the antibody to SEQ ID NO:4.

A variety of immunoassay formats may be used to determine the binding affinity and binding specificity of an antibody with an antigen, such as solid-phase ELISA immunoassays, etc. (Harlow and Lane (1988) Antibodies: A Laboratory Manual, Cold Spring Harbor Publications, New York).

In one embodiment, the antibody that is used as a fusion partner with the invention's proteins (such as one or more of a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II) specifically binds to a cell molecule, preferably to a cell marker molecule, and more preferably to a cell surface marker molecule.

In a further embodiment, the antibody that is used a fusion partner with the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II) is further fused (directly or indirectly) with N-terminal signal peptide, a cell internalization peptide, radionuclide, biotin-binding protein (such as an antibody that is specific for biotin).

In yet a further embodiment, the antibody that is used a fusion partner with the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, and DNase II) specifically binds to proliferating cells, such as endothelial cells and vascular smooth muscle cells that proliferate during angiogenesis; vascular smooth muscle cells that proliferate during restenosis, vascular smooth muscle cells, monocyte cells and macrophage cells that proliferate during atherosclerosis; heart cells, lung cells, and liver cells that proliferate during fibrosis; endothelial cells that proliferate in hemangiomas, psoriasis, retinopathy, macular degeneration, and retinal tearing; leukocyte cells (such as T cells and myeloid cells, etc.), hematopoietic cells, and B cells that proliferate in lymphoma, leukemia, and graft rejection; B cells that proliferate in amyotrophic lateral sclerosis; endothelial cells, synoviocyte cells, and fibroblast cells that proliferate in arthritis; bone cells and synovial cells that proliferate in rheumatoid arthritis and osteoarthritis; skin cells that proliferate in psoriasis and skin cancer; allergen specific antibody secreting cells that proliferate in allergy.

In a further embodiment, the proliferating cells, to which antibodies in fusion molecules of the invention bind, are cancer cells. In a more preferred embodiment, such fusion proteins further comprise one or more of N-terminal signal peptide, cell internalization peptide, and nuclear localization peptide.

Exemplary antibodies that bind to cancer cells include, without limitation, monoclonal antibodies to non-small cell lung carcinomas (U.S. Pat. No. 4,737,579), monoclonal antibodies to human breast cancer (U.S. Pat. No. 4,753,894), monoclonal antibodies to human gastrointestinal cancer (U.S. Pat. No. 4,579,827), and monoclonal antibodies to human renal carcinoma (U.S. Pat. No. 4,713,352).

More particularly, the cancer cells to which the antibody specifically binds comprise liver cancer cells, such as hepatocellular cancer cells. In one embodiment, the antibody that binds to liver cancer cells comprises an antibody chosen from one or more of the antibodies in Table 1, as well as the antibodies Hepama-1, anti-PLC1, anti-PLC2, K-PLC1, K-PLC2, K-PLC2, 49-D6, 7-E10, 34-A4, 26-A10, 34-B9,79-C8,16-E10, 5D3, 5C3, 2C6, a-AFP, HP-1, hHP-1, mAb 95, YPC2/38.8, P215457, PM4E9917, HAb25, HAb27, KY-1, KY-2, KY-3, 9403 Mab, KM-2, S1, 9B2, IB1, A9-84, SF-25, AF-10, XF-8, AF-20, a-hIRS-1, FB-50, SF 31, SF 90, 2A3D2, and 2D11E2, which are further described below.

TABLE 1
Antibody Therapy of HCC.

| Antibody/ | | |
|---|---|---|
| Target antigen | Status | Reference |
| Mab-diphtheria | Active in guinea | Bernhard et al., |
| toxin/290 kD | pig model | 1983, Cancer Res |
| | | 43: 4420-4428 |
| Mab-abrin A chain | Active in guinea | Hwang et al., |
| | pig model | 1984, Cancer Res |
| | | 44: 4578-4586 |
| Mab 336/30 kD | Active in vitro | Stein, et al., |
| | | 1991, Hybridoma |
| | | 10: 255-267 |
| Hepama-1/43 kD | Active in vitro | Fuhrer et al., |
| | | 1991, Cancer Res. |
| | | 51: 2158-2163 |
| Hepama-1-tricho- | Active in vitro | Wang, et al., |
| santhin/43 kD | | 1991, Cancer Res |
| | | 51: 3353-3355 |
| Hepama-1-¹³¹I | Phase I clinical | Zeng, et al., 1994, |
| | trial HAMA | Cancer Immunol Im- |
| | response | munother 39: 332-336 |
| Hepama-1-¹³¹I | Phase II trial | Zeng, et al., 1998, |
| | Improved 5 yr | J Cancer Res Clin |
| | survival | Oncol 124: 275-280 |
| Mab95/40 & 60 kD | Active in vitro | Xie, et al., 1998, |
| | | Hybridoma 17: |
| | | 437-444 |
| Fab′-ping- | Active in animal | Liu, et al., 2001, |
| yangmycin | model | Zhonghua Yi Xue Za |
| | | Zhi 81: 201-201 |
| Hab 18 F(ab′)(2)- | Active in vitro | Yang, et al., 2001, |
| staph enterotoxin | | World J Gastroenterol. |
| A/CD147 | | 7: 216-221 |

In one embodiment, the antibody that may be used in a fusion protein with any one or more of the invention's sequences is Hepama-1, and/or an antibody that specifically binds to the same antigen to which Hepama-1 antibody specifically binds. Data herein shows the use of Hepama-1 in mouse models (Example 12) and clinical studies (Example 13). Hepama-1 was shown to bind specifically to all HCC lines tested, and did not react with other tumor types or any of the normal adult tissues tested. Furthermore, radiolabelled Hepama-1 has shown promising results in clinical trials in China. Therefore, this antibody is an excellent candidate to target the invention's molecules to liver cancer.

Methods for preparing Hepama-1 and testing its specificity to cancer cells are known in the art. Hepama-1 is a mouse monoclonal antibody that was discovered by immunizing mice with human HCC cell line BEL-7402 (Xie et al, Acta Biol. Exp. Sinica 18:263-273, 1985). The antibody showed highly specific and nearly exclusive in vitro reactivity with HCC cell lines and with human primary hepatoma biopsies. The antibody did not bind to other tumor types. The Hepama-1 antigen is a 43,000 dalton glycoprotein, as identified by Western blot. Hepama-1 was converted into an HCC-specific toxin by attaching trichosanthin, a ribosome-inactivating toxin, to it (Wang et al, Cancer Research 51:3353-3355, 1991). This strongly suggests that the Hepama-1 antibody, when attached to its antigen, is internalized by HCC cells.

The Hepama-1 antibody has been conjugated to ¹³¹I and used as a therapeutic agent to combat HCC in humans (Zeng et al, J Cancer Res Clin Oncol 119:257-7, 1993). Twenty-three patients with surgically verified unresectable hepatocellular carcinoma (HCC) have been treated by intrahepatic arterial administration of ¹³¹I-labeled Hepama-1 combined with hepatic artery ligation. Radioimmunoimaging demonstrated that the median tumor/liver ratio was 2.1 (1.1-3.6) at day 5. A decline in alpha-fetoprotein level and shrinkage of tumor were observed in 75% (12/16) and 78% (18/23) of patients respectively. Sequential resection was done in 11 patients (48%) after treatment. The surgical specimens revealed massive necrosis of tumor, but residual cancer cells were found at the edge of the specimens. Anti-antibody was determined in 43% (10/23) of patients 2-4 weeks after the administration of ¹³¹I-Hepama-1 mAb. No marked toxic effects were noted. This work suggested that ¹³¹I-Hepama-1 mAb might be of value as one of the multimodality treatments for unresectable HCC.

Because Hepama-1 is of murine origin, a human anti-murine IgG antibody (HAMA) response was detected in several patients (Zeng it al, Cancer Immunol Immunother 39:332-6, 1994).

Following the initial clinical studies of Zeng et al, a more comprehensive study was undertaken (Zeng it al, J Cancer Res Clin Oncol 124:275-80, 1998). The long-term survival and the prognostic factors in HCC patients treated with radioimmunotherapy were analyzed. Sixty-five patients with surgically verified unresectable HCC were treated with hepatic artery ligation plus hepatic artery cannulation and infusion from 1990 to 1992. Thirty-two patients were enrolled in a phase I-II clinical trial with infusion of ¹³¹I-radiolabelled Hepama-1 mAb via the hepatic artery (the RIT group). Another 33 patients formed the group treated with intrahepatic-arterial chemotherapy (the non-RIT group). T cell subsets were measured in 24 patients and human anti-(murine Ig) antibody (HAMA) were monitored in the RIT group. The 5-year survival rate was significantly higher in the RIT group than in the chemotherapy group, being 28.1% compared to 9.1% (P<0.05); this was mainly a result of better cytoreduction and a higher sequential resection rate (53.1% compared to 9.1%). Significant prognostic factors in the RIT group included tumor capsule status and the number of tumor nodules. HAMA incidence and CD4+ T lymphocytes influenced short-term, but not long-term survival. Thus, intrahepatic-arterial RIT, using ¹³¹I-Hepama-1 mAb, combined with hepatic artery ligation might be an effective approach to improve long-term survival in some patients with unresectable HCC, which may successfully be made resectable by intra-arterial infusion of ¹³¹I-Hepama-1 mAb.

In addition to Hepama-1 monoclonal antibody, the invention also expressly contemplates a fragment of the Hepama-1 monoclonal antibody, humanized Hepama-1 monoclonal antibody, humanized fragments of Hepama-1 monoclonal antibody, and de-immunized Hepama-1 monoclonal antibody. In particular, a humanized monoclonal Hepama-1 antibody is preferred in some applications (such as in human immunotherapy) since murine monoclonal antibodies administered to humans may elicit human immune responses against these molecules. The human anti-mouse antibody (HAMA) responses are directed to two different domains. The responses against the variable region are so called anti-idiotype responses which could block the antigen-binding activity of murine antibodies. The responses against the constant region represent anti-isotype responses, which can block the effector function of antibodies. The HAMA responses not only block the functions of newly administered antibodies but also result in formation of immune complexes with the murine antibodies, which cause some side effects and could reduce the half life of the antibody. Second, the half-life of murine antibodies even in the absence of immune complex formation is much shorter than that of human antibodies in vivo. Third, the effector functions through the Fc region of murine antibodies are weak or non-existent compared to those of human antibodies.

In one embodiment, the antibody that may be used in a fusion protein with any one or more of the invention's sequences is one or more of anti-PLC1 and anti-PLC-2. Methods for preparing these antibodies and testing their specificity to cancer cells are known in the art. For example, Shouval et al (Hepatology 5(3):347-56, 1985) created a library of murine monoclonal antibodies reactive with human hepatoma cells following immunization of Balb/c mice with an intact cloned human hepatoma cell line, designated PLC/PRF/5-NR. One such IgG2a antibody, designated anti-PLC1, specifically stains parental PLC/PRF/5 cell membranes and membranes of SK-Hep 1 and Mahlavu human hepatoma cells grown in culture. A similar pattern of membranous staining was observed in solid tumors derived from the three hepatoma cell lines which were injected subcutaneously into athymic nude rats and mice. Spontaneous capping on the cell surface was observed in 7 to 30% of the three human hepatocellular carcinoma cell types when incubated in suspension with monoclonal anti-PLC1 at 37° C. Treatment of cells with trypsin or sustained growth in culture did not affect the intensity of membranous staining. Monoclonal anti-PLC1 appeared specific, and antibodies did not stain a variety of human carcinoma cell lines and primary tumors of nonhepatic origin, or several normal human and murine tissues. Purified ¹²⁵I-labeled monoclonal anti-PLC1 bound specifically to the three hepatoma cell lines in culture. Specificity of the antigen-antibody reaction was demonstrated by competitive binding inhibition in experiments using unlabeled homologous antibody. Binding of ¹²⁵I-anti-PLC1 was not inhibited by unlabeled monoclonal antibodies to HBsAg or to alpha-fetoprotein. Two hepatoma cell lines secrete a protein that specifically blocks binding of ¹²⁵I-anti-PLC1 antibodies to cell surface antigenic determinants. In this publication, an antibody against another HCC surface protein, designated PLC2, was also identified and is referred to as anti-PLC2.

In a further embodiment, the antibody that may be used in a fusion protein with any one or more of the invention's sequences may be one or more of K-PLC1, K-PLC2 and K-PLC3. Methods for preparing these antibodies and testing their specificity to cancer cells are known in the art. For example, Weidmann et al (Hepatology 7(3):543-50, 1987) identified monoclonal antibodies following immunization of mice with the HBsAg and alpha-fetoprotein-secreting human hepatoma PLC/PRF/5 (“Alexander”) cell line. Three antibodies (K-PLC1, K-PLC2 and K-PLC3) showed evidence of carcinoma-associated reactivity by indirect immunofluorescence. Antibodies K-PLC2 and K-PLC3 reacted only with PLC/PRF/5 cells, but not with any other normal or malignant cell type tested, including the Hep/G2 hepatoma cell line. The reactivity of these antibodies was not removed by absorption with homogenates of either normal liver or a primary hepatocellular carcinoma. These results suggest that K-PLC2 and K-PLC3 identify PLC/PRF/5 idiospecific determinants. Following surface iodination of PLC/PRF/5 cells, immunoprecipitation and analysis on polyacrylamide gels, these specific determinants were found to be of 200,000 and 76,000 daltons, respectively. On the other hand, antibody K-PLC1, although unreactive by immunofluorescence on the majority of normal cell types, including those of lymphoid organs and bone marrow liver cells and most epithelia, was weakly positive on some normal ductal secretory epithelia and was positive on vascular endothelium. However, K-PLC1 reacted strongly with all carcinoma specimens tested, and with most carcinoma-derived cell lines, indicating a large increase in K-PLC1 antigen expression by epithelial cells after malignant transformation. Absorption of K-PLC1 with normal liver homogenate had no affect, but absorption with a hepatocarcinoma homogenate abolished its activity. The K-PLC1 antigen could not be immunoblotted or immunoprecipitated and resolved on polyacrylamide gels; yet it showed the properties of a phospholipid, namely resistance to proteases, extractability with organic solvents and sensitivity to phospholipase C. Using an indirect immunofluorescence technique this antibody produced membrane staining of three hepatocellular carcinoma (HCC) cell lines and it has positively stained 10 of 11 human HCC biopsy specimens (Dunk et al, J Hepatol 4(1):52-61, 1987). In vitro, ¹²⁵I-labelled K-PLC1 binds specifically to PLC/PRF/5 cells, as shown by competitive inhibition experiments. Tumors derived from the PLC/PRF/5 cell line were grown in nude mice and groups of tumor-bearing animals were injected with either (¹²⁵I)K-PLC1 or (¹²⁵I)mouse IgG, and then killed at 1, 4 or 7 days post injection. Bound radioactivity was counted in a variety of solid organs. Tumor:liver ratios for K-PLC1 were greater than those for mouse IgG at each time point, the differences being greatest on day 4 (ratio K-PLC1 4.4+/−0.93, ratio mouse IgG 1.53+/−0.60, mean+/−SD, P less than 0.05). The amount of(¹²⁵I)K-PLC1 bound was greater in the tumor than in any other solid organ, the differences again being maximal on day four. Blood pool radioactivity however remained high throughout the study period.

In another embodiment, the antibody that may be used in a fusion protein with any one or more of the invention's sequences may be one or more of 49-D6, 7-E10, 34-A4, 26-A10, 34-B9,79-C8, and 16-E10. Methods for preparing these antibodies and testing their specificity to cancer cells are known in the art. Other monoclonal antibodies against HCC-specific non-protein cell markers have also been described. Hiraiwa et al Cancer Res 50(10):2917-28, 1990) found an accumulation of sulfated and very complex, highly acidic glycolipids in cultured human hepatocellular carcinoma cells. Among the cells tested, PLC/PRF/5 cells contained a significant amount of very complex sulfated acidic glycolipids, and HepG2 cells were characterized as having a large amount of relatively simple sulfated glycolipids. Several monoclonal antibodies (all IgM) directed to these sulfated and highly acidic glycolipids were established. Among them, 49-D6 and 7-E11 were both directed to SM3 (LacCer-II3-sulfate), a relatively simple sulfated glycolipid, and 34-A4 was directed to SDla (GgOse4CerII 3,IV3-disulfate) and more complex sulfated glycolipids. The other four antibodies, 26-A10, 34-B9, 79-C8, and 16-E10, reacted with unknown highly acidic glycolipids, which were eluted in 0.9-2.7 M ammonium acetate in DEAE chromatography, indicating that these antigenic glycolipids were far more acidic than the usual glycolipids described until now. Analysis of the glycolipids extracted from the hepatocellular carcinoma tissues and cirrhotic livers of patients and from a normal liver with these monoclonal antibodies revealed that sulfated glycolipids having simple carbohydrate structures such as SM3 accumulated significantly in the cirrhotic liver (2 of 4 cases) as well as hepatocellular carcinoma tissue (15 of 17 cases, 88%), and more complex sulfated glycolipids and highly acidic glycolipids were much more specific to hepatocellular carcinoma tissues (10 of 17 cases, 59%) compared to the cirrhotic liver (0 of 4 cases).

In yet another embodiment, the antibody that may be used in a fusion protein with any one or more of the invention's sequences may be one or more of 5D3, 5C3, and 2C6. Methods for preparing these antibodies and testing their specificity to cancer cells are known in the art. There is an association of HCC with hepatitis B infection, and thus the “hepatitis B surface antigen” (HBsAg) is a cell surface marker of some HCC cell types. Therefore, antibodies against HBsAg could target HCC cells in a human host. Examples of three known HBsAg antibodies are 5D3, 5C3, and 2C6 (Shouval et al, Proc Natl Acad Sci, USA 79:650-4, 1982).

In one embodiment, the antibody that may be used in a fusion protein with any one or more of the invention's sequences comprises the antibody a-AFP. Alpha fetoprotein (AFP) is another widely known cell surface marker for some HCC cells. An anti-AFP antibody, herein referred to as a-AFP, has been identified that binds to this marker (Tsung et al, J Immunol Methods 39(4):363-8, 1980).

In one embodiment, the antibody that may be used in a fusion protein with any one or more of the invention's sequences comprises one or more of the antibodies HP-1 and hHP-1. HP-1 is a mouse monoclonal antibody that binds to a cell surface marker on several human hepatoma cell lines. hHP-1 is a humanized version of the same antibody. Both antibodies are described in Chan et al, Biochem. Biophys. Res. Commun 284, 157-167, 2001.

In an alternative embodiment, the antibody that may be used in a fusion protein with any one or more of the invention's sequences comprises the antibody mAb 95. The mouse monoclonal antibody mAb 95 was obtained by immunizing mice with crude cell membranes from the human hepatocellular carcinoma cell line SMMC-7721 (Hybridoma 17(5), 437-444, 1998). The antibody reacts with membrane proteins of apparent molecular weight about 40,000 and 60,000 daltons that are present on HCC cells but not on other cancer cells or normal liver cells.

In a further embodiment, the antibody that may be used in a fusion protein with any one or more of the invention's sequences comprises the antibody UPC2/38.8. A rat monoclonal antibody, YPC2/38.8, was selected from a panel of antibodies derived by immunizing rats with fresh human colorectal carcinoma (Markham et al, J Hepatol 2(1), 25-31, 1986). It was found to bind to a 30,000 dalton protein present on the cell surface of normal colon and liver. This protein was increased 10-fold on primary hepatocellular carcinoma (PHC) cells. After labelling with ¹³¹I, YPC2/38.8 was shown to localize human PHCs grown as xenografts in immunosuppressed mice.

Further embodiments of fusion proteins that contain any one or more of the invention's sequences comprise one or more of the antibodies P215457 and PM4E9917. Monoclonal antibodies P215457 and PM4E9917 were produced by immunization of mice with single cell suspensions of nontrypsin-treated human hepatocellular carcinoma cell (Carlson et al, J Clin Invest 76(1):40-51, 1985). The antibodies were characterized with regards to specificity for hepatoma-associated antigens and their capability for use as reagents in radioimmunoassays (RIAs) and tumor localization in vivo. Two such antibodies namely, P215457 and PM4E9917, of the IgG2a isotype, not only recognized separate and distinct antigenic determinants on four human hepatoma cell lines but also reacted with epitopes present on chemically induced rat hepatoma cell lines. In contrast, only 1 of 38 other human malignant and transformed cell lines demonstrated reactivity with the two antibodies; normal human tissues were also found to be unreactive. Monoclonal antibody P215457 densely stained the plasma membrane by indirect immunofluorescence, showed rapid binding activity to HCC cells in suspension, and precipitated a 50,000-mol wt cell surface protein; antibody PM4E9917 also stained the plasma membrane and precipitated a 65,000-mol wt protein. Also, the Fab fragment of P215457 was found to be useful in tumor localization in vivo.

In yet another embodiment of fusion proteins that contain any one or more of the invention's sequences, the fusion proteins comprise one or more of the antibodies HAb25 and HAb27. Two other monoclonal antibodies that bind to HCC cells are HAb25 (Hu et al, 7(2):101-3, 1999) and HAb27 (Yang et al., 16(4):263-5, 1994). The sequence of the variable regions of these antibodies have been determined, as described in the above publications.

In yet another embodiment of fusion proteins that contain any one or more of the invention's sequences, the fusion proteins comprise one or more of the mouse monoclonal antibodies KY-1, KY-2 and KY-3. These antibodies were identified by immunizing mice with the human hepatocellular carcinoma cell line hu-H2 (Ohzu et all, J Gastroenterol Hepatol 5(6):601-7,1990; Kuwata et al, J Gastroenterol Hepatol 13(2):137-44, 1998). One of these, designated as KY-1, reacted with several HCC lines and a majority of peripheral blood lymphocytes. A second monoclonal antibody, KY-2, reacted only with HCC cell lines but not with others. With this KY-2 antibody, which was highly specific for HCC, HCC tissue was positively stained. A third monoclonal antibody, KY-3, reacted with HCC lines and many other malignant cell lines, but not with non-malignant cells. These results indicate that at least three different tumor-associated molecules are expressed on human HCC.

In a further embodiment of fusion proteins that contain any one or more of the invention's sequences, the fusion proteins comprise an HCC surface marker reactive monoclonal antibody referred to as 9403 Mab (Song et al., Cell Res 8(3), 241-7, 1998).

In another embodiment, the antibody that may be used in a fusion protein with any one or more of the invention's sequences comprises the monoclonal antibodies that react with HCC cell surface markers and that are described in Xie et al, Shi Yan Sheng Wu Xue Bao 18(2):263-70, 1985. Tan (Ann Acad. Med. Singapore 19:147-151, 1990 also described HCC-specific monoclonal antibodies derived from the immunization of mice with human HCC cell line PLC/PRF/5. Seven mouse monoclonal antibodies that react with human HCC cell line SK-HEP-1 were also described (Chang et al, Chung Hua Min Kuo Wei Sheng Wu Chi Mien I Hsueh Tsa Chi 22:1-20, 1989).

In yet another embodiment, the antibody that may be used in a fusion protein with any one or more of the invention's sequences comprises the monoclonal antibody KM-2, which was discovered by immunizing mice with HCC cell line PLC/PRF/5 (Kumagai et al., Cancer Res 52(18):4987-94, 1992). This antibody was used to characterize a new HCC-associated antigen (KM-2 antigen). The KM-2 antigen was strongly expressed on the cell surface of HCC cell lines. Immunofluorescence staining of frozen sections of different tissues and tumors confirmed its specific expression on the cell surface of a group of HCC. The antigen was also detected in the bile canaliculi of normal liver. Its biochemical characterization revealed a high molecular weight (M(r) approximately 900,000) glycoprotein with an N-linked carbohydrate chain close to the peptide epitope recognized by the KM-2 monoclonal antibody.

In an alternative embodiment, the antibody that may be used in a fusion protein with any one or more of the invention's sequences comprises the HCC surface marker-recognizing antibody that is named S1 (Fukuda et al., Cancer Immunol Immunother 27(1):26-32, 1988), and that is an IgG2a that recognizes a carbohydrate moiety and shows antibody-dependent cell (or macrophage)-mediated cytotoxicity (ADCC or ADMC) in conjunction with murine splenocytes of both BALB/c and athymic mice. in vivo experiments demonstrated that the antibody S1 clearly prolonged the survival of athymic mice which had been inoculated with a human liver carcinoma cell line. In addition, the antibody S1 significantly suppressed the human hepatoma line transplanted s.c. into nude mice. ¹²⁵I-Labeled monoclonal antibody S1 revealed that the antibody accumulated significantly in the tumor mass. Many mononuclear cells were observed surrounding tumor cells when the antibody was given. This model system might be useful for analyzing the ADCC (or ADMC) mechanism in vivo.

In a further embodiment, the antibody that may be used in a fusion protein with any one or more of the invention's sequences comprises one or more of the monoclonal antibodies IB1 and 9B2. These antibodies show selectivity for human hepatoma cell lines were produced by immunizing BALB/c mice with human hepatoma cell lines, HA22T/VGH or Hep 3B, and fusing sensitized mouse spleen cells with mouse myeloma cells (Hu et al, Hepatology 6(6):1396-492, 1986). Two monoclonal antibodies recognizing antigens present only on human hepatoma cell lines were investigated. The monoclonal antibody IB1 was found to react with 3 of 9 hepatoma cell lines. Monoclonal antibody 9B2 reacted with all nine hepatoma cell lines. None of the other 20 cell lines tested was bound by IB1 and 9B2. The immunoperoxidase staining of monoclonal antibodies on frozen sections of paired hepatoma and normal liver tissues from the same individuals were studied. Antibody IB1 reacted with 3 of 13 hepatoma tissues, but with none of the normal liver and other tissues, and antibody 9B2 was reactive with antigens appearing on the bile canalicular domain of hepatoma and normal liver tissues. The antibody 9B2 stained no normal tissues with the exception of proximal tubules of kidney. Radioimmunoprecipitation tests identified two antigens reacting with 9B2. The major antigen had an apparent molecular weight of 140,000 and a minor one of 130,000. Therefore, antibody IB1 seems to be specific for antigens present on a group of human hepatoma cells and may be useful for classification and diagnosis of human hepatomas. Antibody 9B2 is quite specific to human liver cells and may be used to provide clues for the characterization of tumor cell lines, identification of metastatic tumors with hepatocytic origin, and study of the structure and function of bile canaliculi.

In yet another embodiment, the antibody that may be used in a fusion protein with any one or more of the invention's sequences comprises monoclonal antibody A9-84 against a hepatocellular carcinoma cell line (PLC/PRF-5). This antibody was also produced by immunization (Shao, Zhonghua Zhong Liu Za Zhi 8(4):259-61, 1986). The specificity of the antibody was studied by enzyme-linked binding assay and immunofluorescence methods. It shows that A9-84 do not respond to 8 different human cancer cell lines (4 liver cancer, 1 esophageal cancer, 1 stomach cancer, 1 multiple myeloma and 1 lymphoblast cell line) and the peripheral mononuclear cells of 91 normal subjects. A9-84 is the subtype of IgG3. It is capable of inhibiting the growth of cultured PLC/PRF/5 cells with or without complement.

In a further embodiment, the antibody that may be used in a fusion protein with any one or more of the invention's sequences comprises SF-25 (Cancer Res 48(22):6573-9, 1988). This antibody was produced against a human hepatoma cell line (FOCUS) that strongly reacts with an antigen shared by all six colon adenocarcinoma cell lines. This cell surface antigen was uniformly expressed in all 17 human adenocarcinomas of the colon obtained at surgery but not on the normal adjacent mucosa counterpart. Other normal tissues were negative except for a population of cells in the distal tubule of the kidney as shown by immunoperoxidase staining and direct binding to membrane preparations. Binding of this Mr 125,000 antigen to antibody is disrupted by detergents, sodium dodecyl sulfate, and paraformaldehyde fixation but not by treatment of FOCUS cells with trypsin. The SF-25 antibody when labeled with ¹²⁵I shows a striking capacity by both biodistribution and nuclear imaging studies to localize human colon adenocarcinoma grown as solid tumors in nude mice. SF-25 may be useful in distinguishing between normal colon and the transformed phenotype.

In a further embodiment, the antibody that may be used in a fusion protein with any one or more of the invention's sequences comprises AF-10 (Takahashi et al. Cancer Res 49(6): 1349-56, 1989). The mature antigen is a cell surface glycoprotein with a core polypeptide with a molecular weight of 75,000 bearing N-glycosylation units. This protein migrates in sodium dodecyl sulfate-polyacrylamide gel electrophoresis with an apparent molecular weight of 100,000-115,000 in reducing conditions and Mr 115,000-130,000 in nonreducing conditions. The epitope recognized by monoclonal antibody AF-10 is borne by the core protein. The antigen is also expressed on adenocarcinoma of the lung.

In a further embodiment, the antibody that may be used in a fusion protein with any one or more of the invention's sequences comprises one or more of monoclonal antibodies XF-8 and AF-20 that are uniformly present on 15/15 hepatocellular carcinomas tested (Takahashi et al, Hepatology 9(4): 625-34, 1989). Most if not all tumor cells highly express these antigens. Such antigens were not evident on adjacent normal liver and the XF-8 epitope was not found on other normal human tissues. AF-20 antigen distribution revealed low-level expression on a subpopulation of cells in the zona glomerulosa of the adrenal gland and on crypt cells of the small intestinal tract. They studied the capability of radiolabeled XF-8 and AF-20 monoclonal antibodies when administered either alone or in combination to localize a hepatitis B virus-related hepatocellular carcinoma cell line (FOCUS) grown as subcutaneous tumors in nude mice. Biodistribution experiments demonstrated an excellent localization to tumor of 15 to 22% of the injected dose of ¹²⁵I-labeled antibodies. Indeed, it was possible to enhance the delivery of ¹²⁵I to the tumor cell surface by the use of XF-8 and AF-20 in combination. Nuclear imaging studies showed sharp visualization of tumor and demonstrate that these monoclonal antibodies have sufficient specificity and sensitivity to be strongly considered as immunotargeting agents.

In a further embodiment, the antibody that may be used in a fusion protein with any one or more of the invention's sequences comprises the AF-20 antibody, which was used to target HCC cells with an adenoviral gene delivery vector (Yoon et al, Biochem Biophys Res Commun 272(2):497-504, 2000). The authors developed a specific adenoviral gene delivery system with monoclonal antibody (mAb) AF-20 that binds to a 180 kDa antigen highly expressed on human hepatocellular carcinoma (HCC) cells. A bifunctional Fab-antibody conjugate (2Hx-2-AF-20) was generated through AF-20 mAb crosslinkage to an anti-hexon antibody Fab fragment. The high-affinity mAb, AF-20, recognizes a rapidly internalized 180-kd cell-surface glycoprotein that is abundantly expressed on HCC and other human tumors. Uptake of adenoviral particles and gene expression was examined in FOCUS HCC and NIH 3T3 cells by immunofluorescence; beta-galactosidase expression levels were determined following competitive inhibition of adenoviral CAR receptor by excess fibre knob protein. The chimeric complex was rapidly internalized at 37° C., and enhanced levels of reporter gene expression was observed in AF-20 antigen positive HCC cells, but not in AF-20 antigen negative NIH 3T3 control cells. The AF-20 antibody was also used to target immunoliposomes to HCC cells (Moradpour et al, Hepatology 22(5):1527-37, 1995). Immunoliposomes were produced by covalently coupling AF-20 to liposomes containing carboxyfluorescein. Interaction of immunoliposomes with various HCC cell lines in vitro was quantitatively assessed by flow cytometry and qualitatively analyzed by fluorescence microscopy. Liposomes bearing an isotype-matched nonrelevant monoclonal antibody (MAb) and cell lines not expressing AF-20 antigen served as controls. AF-20-immunoliposomes specifically bound to HCC and other human cancer cell lines expressing the AF-20 antigen and were rapidly internalized at 37° C. Interaction of AF-20-conjugated liposomes with these cell lines was between 5 and 200 times greater than that of unconjugated liposomes, whereas no difference was observed between control liposomes bearing a nonrelevant antibody and unconjugated liposomes. Specificity of liposome-target cell interaction was confirmed by competitive inhibition assays. Kinetic analysis showed rapid association of AF-20 immunoliposomes with target cells, with saturation conditions being reached after 60 minutes. Thus, the MAb AF-20 directs highly efficient, specific, and rapid targeting of immunoliposomes to human HCC and other human cancer cell lines in vitro. This targeted liposomal delivery system represents a promising approach for the development of immunotargeted diagnosis and therapy strategies against HCC.

In a further embodiment, the antibody that may be used in a fusion protein with any one or more of the invention's sequences comprises the a-hIRS-1 antibody which binds antigens on the surface of HCC cells (Nishiyama and Wands, Biochem Biophys Res Commun 183(1):280-5, 1992) is the human insulin receptor substrate-1 (hIRS-1), which was cloned from a lambda GT11 expression library using a monoclonal antibody (herein referred to as a-hIRS-1) produced against a human hepatocellular carcinoma (HCC) cell line (FOCUS). It has multiple potential phosphorylation sites, that suggest an intrinsic function of this molecule in response to insulin action, were highly conserved between the two species. A c.a. 180 kDa hIRS-1 protein was immunoprecipitated and found to be phosphorylated on tyrosine residue(s) following insulin stimulation of HuH-7 HCC cells. Northern blot analysis demonstrated a single c.a. 5 kb transcript in HCC cell lines and tissues. Higher levels of HIRS-1 gene transcripts were observed in HCC tumors compared to adjacent non-involved normal liver.

In a further embodiment, the antibody that may be used in a fusion protein with any one or more of the invention's sequences comprises FB-50 (Lavaissiere et al, J Clin Invest 98(6):1313-23, 1996). FB-50, reacts with an antigen that was highly expressed in 4 of 10 primary hepatocellular carcinomas, in all 20 cholangiocarcinomas studied, and in a variety of transformed cell lines. This antigen was also highly expressed in neoplastic epithelial cells of breast and colon carcinomas in contrast to its low level of expression in normal hepatocytes and in non-neoplastic epithelial cells. Among the normal adult tissues studied, high levels were observed only in proliferating trophoblastic cells of the placenta and in adrenal glands. A 636-bp partial cDNA, isolated from a gamma GT11 expression library generated with HepG2 human hepatoblastoma cells, and a complete cDNA, generated by reverse transcriptase-PCR, identified the antigen as the human form of aspartyl(asparaginyl)beta-hydroxylase. This enzyme catalyzes posttranslational hydroxylation of beta carbons of specific aspartyl and asparaginyl residues in EGF-like domains of certain proteins. Analyses of extracts prepared from several human tumor cell lines compared to their normal tissue counterparts indicate that the increase in hydroxylase, approximately 10-fold, is controlled at the level of transcription and the protein is expressed in an enzymatically active form. In similar analyses, comparing hepatocellular carcinomas to adjacent uninvolved liver from five patients, enzymatic activity was much higher in the tumor tissue from the four patients whose immunoblots revealed increased hydroxylase protein in the malignant tissue.

In a further embodiment, the antibody that may be used in a fusion protein with any one or more of the invention's sequences comprises one or more of SF 31 and SF 90(Ozturk et al, 49(23): 6764-73, 1989). The antibodies were used to identify a Mr 50,000 cell surface protein antigen (p50) on a human hepatocellular carcinoma derived cell line (FOCUS). This antigen was subsequently shown to be expressed in vivo in human hepatocellular carcinoma. All 18 tumors tested by Western immunoblotting demonstrated high levels of p50 with undetectable amounts observed in the adjacent normal liver counterparts. Further characterization revealed that p50 is a monomeric polypeptide with a neutral pI (6.5-7.2) and appears not to be glycosylated. The cellular localization was determined by direct antibody binding to intact cells, immunoprecipitation of ¹²⁵I-labeled cell surface proteins, and Western immunoblotting of subcellular fractions. p50 was found on the cell surface as well as in the cytoplasm. in vitro monoclonal antibody binding studies indicate that the protein is expressed in all human malignant cells (n=34) tested thus far regardless of the embryonic tissue of origin and the degree of differentiation. p50 was present at very low levels in normal tissues with the notable exception of high expression in adrenal glands. The protein is conserved in mammalian evolution since a similar protein was also found in bovine adrenals. The molecular characteristics and the pattern of expression of p50 indicate that this normal adrenal protein is associated with the transformed phenotype.

In a further embodiment, the antibody that may be used in a fusion protein with any one or more of the invention's sequences comprises one or more of 2A3D2 and 2D11E2. These are murine IgM's against HCC (Hiraiwa et al. Cancer Res 50(17):5497-503, 1990) and are directed to the gangliosides and sialoglycoproteins related to a rare blood group antigen, Cad, were obtained by using a ganglioside mixture prepared from human hepatocellular carcinoma cells (PLC/PRF/5) as the immunogen. These two monoclonal antibodies detected multiple ganglioside antigens present in the PLC/PRF/5 cells, and the major antigenic ganglioside was characterized as IV4GalNAc beta-GD1a, which has the carbohydrate structure GalNAc beta 1----4(NeuAc alpha 2----3)Gal beta 1----3GalNAc beta 1----4(NeuAc alpha 2----3)Gal beta 1----Cer. The two antibodies also reacted with GM2 (GalNAc beta 1----4(NeuAc alpha 2----3)Gal beta 1----4Glc beta 1----Cer) and a Cad-active lactoseries ganglioside (IV4GalNAc beta-sialosylparagloboside, GalNAc beta 1----4(NeuAc alpha 2----3)Gal beta 1----4GlcNAc beta 1----3Gal beta 1----4Glc beta 1----Cer), which have carbohydrate structures related to IV4GalNAc beta-GD1a. Beside gangliosides, both antibodies recognized the carbohydrate determinant carried by glycophorin A on very rare Cad-positive human RBC; the structure of which is GalNAc beta 1----4(NeuAc alpha 2----3)Gal beta 1----3(NeuAc alpha 2----6)GalNAc alpha 1----Ser/Thr. From these findings, it is clear that monoclonal antibodies 2A3D2 and 2D11E2 both recognize the nonreduced carbohydrate terminus composed of three sugar residues, GalNac beta 1----4(NeuAc alpha 2----3)Gal beta 1----R, and are useful for detecting the Cad-related antigen in cells and tissues. By using these monoclonal antibodies, it was revealed that many cultured human hepatocellular carcinoma cell lines and cancer tissues taken from patients with hepatocellular carcinoma contain both Cad-active glycoprotein antigens and related gangliosides, while normal liver tissues contain no appreciable amount of either species of antigen. The Cad-active glycoprotein antigens in cultured human hepatocellular carcinoma cells appeared as triplet bands having molecular weights of 92,000, 75,000, and 61,000, under either reducing or nonreducing conditions in sodium dodecyl sulfate-polyacrylamide gel electrophoresis. Essentially the same triplet proteins were observed in as many as 4 of 9 cases (44%) of cancer tissue from patients with hepatocellular carcinoma, but not in neighboring cirrhotic tissues or normal liver tissues.

The invention is not limited to antibodies that are specific for liver cancer cells, but also includes antibodies (and other molecules) that specifically bind with any cancer, such as B cell lymphoma, myeloid leukemia, renal carcinoma, colon cancer, pancreatic cancer, colorectal cancer, breast cancer, ovarian cancer, prostate cancer.

Exemplary antibodies that may be useful in the invention's conjugates, and that have been shown to specifically bind with tumor cells include those described in Hellstrom et al., Proc. Natl. Acad. Sci. USA 83:7059-7063 (1986), Drebin et al., Oncogene 2:387-394 (1988), Papsidero, Semin. Surg. Oncol. 1(4):171-81 (1985); Schlom et al., Important Adv. Oncol., 170-92 (1985); Allum et al., Surg. Ann. 18:41-64 (1986); and Houghton et al., Semin. Oncol., 13(2):165-79 (1986).

Additional exemplary cancers, the nature of the target molecule on the cancer cell, and of the molecule (including antibodies) that specifically bind to these targets are illustrated in the following Table 2.

TABLE 2
Examples Antibodies For Targeting The
Invention's Molecules to Cells

| | | Targeting | |
|---|---|---|---|
| Disease | Target | Molecule | Reference |
| B cell | B cell | Anti-idio- | Vuist et al., |
| lymphoma | receptor | type mAb | 1994, Blood 83: |
| | | | 899-906; Davis |
| | | | et al., 1998, |
| | | | Blood 92: 1184- |
| | | | 1190. |
| ″ | CD22 | Anti-CD22 | Ghetie et al., |
| | | mAb | 1997, Mol Med 3: |
| | | | 420-427. |
| Myeloid | CD33 | Anti-CD33 | |
| leukemia | | mAb | |
| Renal car- | Renal γ- | mAb 138H11 | Knoll et al., |
| cinoma | glutamyl- | | 2000, Cancer Res |
| | transferase | | 60: 6089-6094. |
| Colon and | mucin-type | mAb C242 | Liu et al., 1996, |
| pancreatic | glycoprotein | | Proc NatlAcad Sci |
| cancer | | | 93: 8618-8623; |
| | | | Giantonio et al., |
| | | | 1997, J Clin Oncol |
| | | | 15: 1994-2007. |
| Colon cancer | Transferrin | Fv fragment | Shinohara et al., |
| | receptor | of mAb HB21 | 2000, Int J Oncol |
| | | | 17: 643-651. |
| Colorectal | Carcinoem- | mAb hMN14 | Akamatsu et al., |
| cancer | bryonic | | 1998., Clin Cancer |
| | Antigen | | Res 4: 2825- |
| | | | 2832. |
| Colon cancer | Lewis(y) | mAb BR96 | Sjogren et al., |
| | antigen | | 1997, Cancer Res |
| | | | 57: 4530-4536. |
| Colon and | Lewis(y) | mAb B3 | Pai et al., 1996, |
| breast | antigen | | Nat Med 2: 350- |
| cancer | | | 353. |
| Colon cancer | 72 kD colon | mAb | Byers et al., 1989, |
| | cancer | 791T/36 | Cancer Res 49: |
| | Antigen | | 6153-6160. |
| Breast and | p185^{HER-2} | mAb TA1 | Xu et al., 2000, |
| ovarian | | | Clin Cancer Res 6: |
| cancer | | | 3334-3341. |
| Breast cancer | erbB2 | mAb e23 | Pai-Scherf et al., |
| | | | 1999, Clin Cancer |
| | | | Res 5: 2311- |
| | | | 2315. |
| Breast cancer | Lewis(y) | mAb BR96 | Tolcher et al., |
| | antigen | | 1999, J Clin Oncol |
| | | | 17: 478-484. |
| Prostate | Prostate | mAb 8D11 | Ross et al., 2002, |
| cancer cell | stem | | Cancer Res 62: |
| antigen | | | 2546-2553. |
| Prostate cancer | E-selectin | mAb vs E- | Bhaskar et al., |
| | | selectin | 2003, Cancer Res |
| | | | 63: 6387-6394.6 |
| Prostate cancer | bFGF | bFGF | Davol et al., |
| | receptor | | 1999, Prostate |
| | | | 40: 178-191. |
| Prostate and | Lutenizing | LHRH receptor | Gho et al., Mol |
| breast cancer | hormone | | Cells 9: 31-36. |
| | releasing | | |
| | hormone | | |
| | (LHRH) | | |
| Lung cancer | IL-4 | IL-4 | Kawakami et al., |
| | receptor | | 2002, Clin Cancer |
| | | | Res 8: 3503- |
| | | | 3511.19 |
| Medullo- | IL-4 | IL-4 | Joshi et al., 2002, |
| blastoma | receptor | | Br J Cancer 86: |
| | | | 285-291. |
| Breast cancer | Somatostatin | Somatostatin | Kahan et al., 1999, |
| | Receptor | | Int J Cancer 82: |
| | | | 592-598. |
| Angiogenesis | Endoglin | mAb K4-2C10 | Seon et al., 1997, |
| cancer- | | vs endoglin | Clin Cancer Res 3: |
| related | | | 1031-1044. |
| Angiogenesis | Vascular | VEGF | Arora et al., 1999, |
| cancer- | endothelial | | Cancer Res 59: |
| related | growth | | 183-188. |
| | factor (VEGF) | | |
| | receptor | | |

In addition to the specifically recited antibodies, other antibodies that may be used in a fusion protein with any one or more of the invention's sequences comprises an antibody that specifically binds to the same antigen as that which is specifically bound by any one of the above-described liver cancer specific mouse monoclonal antibodies Hepama-1, anti-PLC1, anti-PLC2, K-PLC1, K-PLC2, K-PLC3, 49-D6, 7-E10, 34-A4, 26-A10, 34-B9,79-C8,16-E10, 5D3, 5C3, 2C6, a-AFP, HP-1, hHP-1, mAb 95, YPC2/38.8P215457, PM4E9917, HAb25, HAb27, KY-1, KY-2, KY-3, 9403 Mab, KM-2, S1, 9B2, IB1, A9-84, SF-25, AF-10, XF-8, AF-20, a-hIRS-1, FB-50, SF 31, SF 90, 2A3D2, and 2D11E2.

Other antibodies within the scope of the invention include those that specifically bind to B cell lymphoma, myeloid leukemia, renal carcinoma, colon cancer, pancreatic cancer, colorectal cancer, breast cancer, ovarian cancer, prostate cancer. These are exemplified by anti-idiotype mAb which specifically binds B cell receptor, anti-CD22 mAb which specifically binds CD22, anti-CD33 mAb which specifically binds CD33, mAb 138H11 which specifically binds Renal γ-glutamyl-transferase, mAb C242 which specifically binds mucin-type glycoprotein, Fv fragment of mAb HB21 which specifically binds Transferrin receptor, mAb hMN14 which specifically binds Carcinoembryonic antigen, mAb BR96 and mAb B3 which specifically bind Lewis(y) antigen, mAb 791T/36 which specifically binds 72 kD colon cancer antigen, mAb TA1 which specifically binds p185^{HER2}, mAb e23 which specifically binds erbB2, mAb 8D11 which specifically binds Prostate stem cell antigen, and mAb vs E-selectin which specifically binds E-selectin.

These antibodies may bind to cancer cells competitively or simultaneously with any antibody from the above-described antibodies. The simultaneous binding can be shown by immunoprecipitation of the cell surface marker-recognizing compound by any antibody from the group of antibodies in the presence of solubilized cell surface proteins from cancer cells.

In a preferred embodiment the antibodies (such as a monoclonal antibody or an antibody fragment like a Fab, Fab′, F(ab′)2 or a scFv) that specifically bind to liver cancer cells are those that specifically bind with a protein of molecular weight of about 43,000 daltons. This liver cell surface protein might be a glycoprotein.

In a more preferred embodiment, the antibodies (such as a monoclonal antibody or an antibody fragment like a Fab, Fab′, F(ab′)2 or a scFv) that specifically bind to liver cancer cells are those that bind to the same antigen as the Hepama-1 monoclonal antibody, and preferably with higher affinity than the Hepama-1 antibody. Such antibodies may be obtained by using monoclonal antibody technology including mouse monoclonal or rabbit monoclonal technology, by using mRNA display, and/or ribosome display.

A preferred way to obtain the protein-binding agent is through determining the identity of the Hepama-1 target, purifying the Hepama-1 target, and creating a binding agent against the purified Hepama-1 target. Another preferred way to obtain the protein-binding agent is through determining the identity of the Hepama-1 target, synthesizing a peptide comprising the extracellular peptide portion of the Hepama-1 target, creating a binding agent against the peptide, and confirming that the binding agent against the peptide can recognize the Hepama-1 antigen on hepatocellular carcinoma cells with a higher affinity than the Hepama-1 antibody.

8. Conjugates Containing Ligands Of Cell Receptors

In yet another embodiment, conjugates that contain the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II) and that are within the scope of the invention comprise a ligand of a cell receptor (including intracellular receptors and cell surface receptors, and more preferably cell surface receptors). As used herein the term “ligand” refers to a molecule that binds to a second molecule. A particular molecule may be referred to as either, or both, a ligand and second molecule. Examples of second molecules include a receptor of the ligand, and an antibody that binds to the ligand. Thus, the term “ligand of a cell receptor” refers to a molecule that binds to a cell receptor.

In one embodiment, the ligand is chosen from one or more of a peptide (including a constrained peptide), antibody (including a single domain antibody, a diabody, etc.), and a partially randomized protein based on a known structural motif, on the structure of a lens crystalline or of fibronectin. In another embodiment, the ligand of a cell receptor comprises a growth factor, such as one or more of epidermal growth factor, insulin-like growth factor, fibroblast growth factor, and vascular endothelial growth factor. In a particularly preferred embodiment, the growth factor comprises basic fibroblast growth factor SEQ ID NO:70 (GenBank No. AAA52533) encoded by CDS 467-934 of the nucleic acid sequence (SEQ ID NO:71) (GenBank No. J04513.1). The exemplary basic fibroblast growth factor SEQ ID NO:70 represents that 18 kD human basic fibroblast growth factor (bFGF) that was fused to recombinant ADF (rADF) as the fusion protein rADF-bFGF, and that was shown to be toxic to tumor cells (Example 6).

In another embodiment the conjugates that contain the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II) as well as contain a ligand of a cell receptor, further comprise one or more of a N-terminal signal peptide, a cell internalization peptide, a nuclear localization peptide, a radionuclide, a biotin-binding protein (such as an antibody that is specific for biotin).

9. Conjugates Containing Aptamers

In yet another embodiment, conjugates that contain the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase 11) and that are within the scope of the invention comprise an aptamer. As used herein the term “aptamer” refers to single-stranded oligonucleotide sequences that bind to target molecules, such as small molecule ligands and proteins. Methods for making and using aptamers are known in the art (U.S. Pat. No. 6,511,809 to Baez et al., issued Jan. 28, 2003; U.S. Pat. No. 6,509,460 to Beigelman et al., issued Jan. 21, 2003; and Mol Diagn 1999 December;4(4):381-8; Marshall et al, Current Biology, 5, 729-734 (1997)).

Optionally, conjugates that contain an aptamer, in addition to containing the invention's proteins (such as a portion of MDH that comprises one or more of MADF and ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II), may further contain one or more of a N-terminal signal peptide, a cell internalization peptide, a nuclear localization peptide, a radionuclide, and a biotin-binding protein (such as an antibody that is specific for biotin).

C. Nucleic Acid Sequences of the Invention

The invention provides a composition comprising a nucleic acid sequence encoding an amino acid sequence that comprises a MDH portion, MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II. The term “nucleic acid molecule” includes RNA and DNA (such as cDNA).

The invention's nucleic acid sequences are useful as, for example, probes for detecting (such as by using Southern blot hybridization) the presence of nucleic acid sequences that encode the invention's proteins (such as MDH portions, MDAF, ADF, etc.). The invention's nucleic acid sequences are also useful as primers (such as in polymerase chain reactions (PCR)) for amplifying nucleic acid sequences that encode the invention's proteins. The invention's nucleic acid sequences are also useful for recombinantly expressing the invention's proteins.

For example, cDNA or genomic libraries of various types may be screened as natural sources of the nucleic acids of the present invention, or such nucleic acids may be provided by amplification of sequences resident in genomic DNA or other natural sources, e.g., by PCR. The choice of cDNA libraries normally corresponds to a tissue source which is abundant in mRNA for the desired proteins. Phage libraries are normally preferred, but other types of libraries may be used. Clones of a library are spread onto plates, transferred to a substrate for screening, denatured and probed for the presence of desired sequences.

The nucleic acid fragments of the present invention may be used to isolate cDNAs and genes encoding homologous proteins from the same or other species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding ADF homologs, either as cDNAs or genomic DNAs, could be isolated directly by using all or a portion of the instant nucleic acid fragments as DNA hybridization probes to screen libraries from any desired organism employing methodology well known to those skilled in the art. Specific oligonucleotide probes based upon the instant nucleic acid sequences can be designed and synthesized by methods known in the art (Current Protocols in Molecular Biology, ed. F. Ausubel et al, John Wiley & Sons, New York, 2000). Moreover, the entire sequences can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al., (1988) PNAS USA 85:8998) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., (1989) PNAS USA 86:5673; Loh et al., (1989) Science 243:217). Products generated by the 3′ and 5′ RACE procedures can be combined to generate full-length cDNAs (Frohman, M. A. and Martin, G. R., (1989) Techniques 1:165).

Availability of the invention's nucleotide sequences and amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lemer, R. A. (1984) Adv. Immunol. 36:1; Current Protocols in Molecular Biology, ed. F. Ausubel et al, John Wiley & Sons, New York, 2000).

Reference herein to any specifically named nucleotide sequence (such as SEQ ID NO:5 (GenBank: NP-005909), SEQ ID NO:9 (GenBank: NM_{—}145074), SEQ ID NO:11 (GenBank: AF100928), SEQ ID NO:13 (GenBank: AF298770), SEQ ID NO:15 (GenBank: BC026033), SEQ ID NO:17 (Bcl-2 GenBank: M14745), SEQ ID NO:19 (GenBank: NM_{—}138764), SEQ ID NO:21 (GenBank:AF031523), SEQ ID NO:23 (GenBank: AF250233), SEQ ID NO:25 (GenBank: NM_{—}004435), SEQ ID NO:27 (GenBank: AB013918), SEQ ID NO:29 (GenBank: BC007112), SEQ ID NO:31 (GenBank: NM_{—}001963), SEQ ID NO:33 (GenBank: AY047581), SEQ ID NO:35 (GenBank: NM_{—}001885), SEQ ID NO:37 (GenBank: M20704), SEQ ID NO:39 (GenBank: BT006856), SEQ ID NO:41 (GenBank: AJ298844), SEQ ID NO:43 (GenBank: AF060222), SEQ ID NO:45 (GenBank: U10421), SEQ ID NO:47 (GenBank: NM_{—}000612), SEQ ID NO:49 (GenBank: NM_{—}033137), SEQ ID NO:51 (GenBank: AY463230), SEQ ID NO:73 (GenBank: AY339584), SEQ ID NO:75 (GenBank: AF452712), SEQ ID NO:77 (GenBank: AF002697), and SEQ ID NO:79 (GenBank: AF544398) that encodes any one of the invention's proteins, etc.), includes within its scope any and all equivalent fragments thereof, homologs thereof, and sequences that hybridize under highly stringent and/or medium stringent conditions to the specifically named nucleotide sequence. In one embodiment, these equivalents have at least one of the biological activities (such as those disclosed herein and/or known in the art) of the specifically named nucleotide sequence, wherein the biological activity is detectable by any method.

The “fragment” or “portion” of a nucleotide sequence may range in size from an exemplary 5, 10, 20, 50, or 100 contiguous nucleotide residues to the entire nucleic acid sequence minus one nucleic acid residue. Thus, a nucleic acid sequence comprising “at least a portion of” a nucleotide sequence comprises from five (5) contiguous nucleotide residues of the nucleotide sequence to the entire nucleotide sequence.

For example, with respect of mitochondrial malate dehydrogenase portion, minimum activator of DNA fragmentation protein, and activator of DNA fragmentation protein, those portions of SEQ ID NO:5 (GenGenBank: NP_{—}005909) that encode these proteins, and that also lack (1) one 5′-nucleic acid, (2) two 5′-nucleic acids, (3) three 5′-nucleic acids, (4) four 5′-nucleic acids, (5) five 5′-nucleic acids, (6) six 5′-nucleic acids, (6) six 5′-nucleic acids, (7) one 3′-nucleic acid, (8) two 3′-nucleic acids, (9) three 3′-nucleic acids, (10) four 3′-nucleic acids, (11) five 3′-nucleic acids, (12) six 3′-nucleic acids, (13) seven 3′-nucleic acids, (13) one 5′-nucleic acid and one 3′-nucleic acid, (14) two 5′-nucleic acids and one 3′-nucleic acid, (15) three 5′-nucleic acids and one 3′-nucleic acid, (16) four 5′-nucleic acids and one 3′-nucleic acid, (17) one 5′-nucleic acid and two 3′-nucleic acids, (18) one 5′-nucleic acid and three 3′-nucleic acids, (19) one 5′-nucleic acid and four 3′-nucleic acids, (20) one 5′-nucleic acid and five 3′-nucleic acids, (21) one 5′-nucleic acid and six 3′-nucleic acids, (22) one 5′-nucleic acid and seven 3′-nucleic acids, (23) two 5′-nucleic acids and two 3′-nucleic acids, (24) two 5′-nucleic acids and three 3′-nucleic acids, (25) three 5′-nucleic acids and four 3′-nucleic acids, (26) one 5′-nucleic acid and four 3′-nucleic acids, (27) two 5′-nucleic acids and six 3′-nucleic acids, (28) three 5′-nucleic acids and two 3′-nucleic acids, (29) six 5′-nucleic acid and five 3′-nucleic acids, and (30) five 5′-nucleic acid and seven 3′-nucleic acids.

In another example, the invention further encompasses illustrative portions of Htra/Omi (SEQ ID NO:9), apoptosis inducing factor (SEQ ID NO:11), Smac/DIABLO (SEQ ID NO:13), EndoG (SEQ ID NO:25), Cytochrome C (SEQ ID NO:73) Nix (SEQ ID NO:75), Nip3 (SEQ ID NO:77), CIDE-B (SEQ ID NO:79, GenBank: AF544398), gelsolin (SEQ ID NO:15) Bcl-2 (SEQ ID NO:17), Bax (SEQ ID NO:19), Bad (SEQ ID NO:21), Bid (SEQ ID NO:23), caspase-activated DNase (SEQ ID NO:27), DNase I (SEQ ID NO:41), DNase II (SEQ ID NO:43), inhibitor of CAD nuclease (SEQ ID NO:29), epidermal growth factor (SEQ ID NO:31), vascular endothelial growth factor (SEQ ID NO:33), lens crystalline protein (SEQ ID NO:35), antennapedia protein (SEQ ID NO:37), fibronectin type 1 (SEQ ID NO:39), human HOX protein (SEQ ID NO:46), insulin-like growth factor (SEQ ID NO:47), fibroblast growth factor (SEQ ID NO:49), and HIV Tat protein (SEQ ID NO:51), that lack (1) one 5′-nucleic acid, (2) two 5′-nucleic acids, (3) three 5′-nucleic acids, (4) four 5′-nucleic acids, (5) five 5′-nucleic acids, (6) six 5′-nucleic acids, (6) six 5′-nucleic acids, (7) one 3′-nucleic acid, (8) two 3′-nucleic acids, (9) three 3′-nucleic acids, (10) four 3′-nucleic acids, (11) five 3′-nucleic acids, (12) six 3′-nucleic acids, (13) seven 3′-nucleic acids, (13) one 5′-nucleic acid and one 3′-nucleic acid, (14) two 5′-nucleic acids and one 3′-nucleic acid, (15) three 5′-nucleic acids and one 3′-nucleic acid, (16) four 5′-nucleic acids and one 3′-nucleic acid, (17) one 5′-nucleic acid and two 3′-nucleic acids, (18) one 5′-nucleic acid and three 3′-nucleic acids, (19) one 5′-nucleic acid and four 3′-nucleic acids, (20) one 5′-nucleic acid and five 3′-nucleic acids, (21) one 5′-nucleic acid and six 3′-nucleic acids, (22) one 5′-nucleic acid and seven 3′-nucleic acids, (23) two 5′-nucleic acids and two 3′-nucleic acids, (24) two 5′-nucleic acids and three 3′-nucleic acids, (25) three 5′-nucleic acids and four 3′-nucleic acids, (26) one 5′-nucleic acid and four 3′-nucleic acids, (27) two 5′-nucleic acids and six 3′-nucleic acids, (28) three 5′-nucleic acids and two 3′-nucleic acids, (29) six 5′-nucleic acid and five 3′-nucleic acids, and (30) five 5′-nucleic acid and seven 3′-nucleic acids.

The term “homolog” when in reference to a specifically named nucleotide sequence or a specifically named amino acid sequence (such as MDH portion, MADF, ADF, Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, fibroblast growth factor, and HIV Tat protein), refers to a nucleotide sequence and amino acid sequence, respectively, which exhibits at least 50% identity, at least 65% identity, at least 70% identity, at least 75% identity, at least 80% identity, at least 85% identity, at least 90% identity, and/or at least 95% identity to the specifically named nucleotide sequence or to the specifically named amino acid sequence, respectively. To determine the level of homology (i.e., percent identity) of two nucleotide sequences or of two amino acid sequences, the two sequences are aligned for optimal comparison purposes (e.g., gaps can be introduced in one or both of the two nucleotide sequences and the two amino acid sequences for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. Optimal alignment of sequences for comparison may be conducted by computerized implementations of known algorithms (e.g., GAP, BESTFIT, FASTA, and TFASTA in the Wisconsin Genetics Software Package, Genetics Computer Group (GCG), 575 Science Dr., Madison, Wis., or BlastN and BlastX available from the National Center for Biotechnology Information), by using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4, or by inspection. Sequences are typically compared using either BlastN or BlastX with default parameters.

Equivalent homologs of the invention's nucleotide sequences may also be identified using Southern hybridization as described, for example, in “Cloning and Sequence” (complied under the supervision of Itaru Watanabe, edited by Masahiro Sugiura, 1989, published by Noson Bunka-sha), using DNA having the base sequence of a known gene as a probe. The gene may be DNA having the base sequence of the known gene or DNA having a base sequence with the addition, deletion or replacement of one or more bases in the DNA of the known gene. For example, double-stranded DNA is dissociated into the complementary single-stranded DNA strands by heat treatment at 95° C. for 1 minute or by alkali treatment with 0.5 M NaOH, 1.5 M NaCl, which are then left cooling on ice for 1 minute or subjected to neutralization with 0.5 M Tris-HCl (pH 7.0), 3.0 M NaCl, so as to associate with single-stranded DNA or single-stranded RNA, which is complementary to the above single-stranded DNAs, to fall into a double-stranded state (i.e., hybridized state) again. Such DNA may be usually a gene having a base sequence with a high homology (e.g., about 90% or higher homology as a whole, although it may vary depending upon whether the region is closely related to an active site or a structure) to the base sequence of the known gene.

The nucleic acid and protein sequences of the present invention can further be used as a “query sequence” to perform a search against sequence databases to, for example, identify other family members or related sequences. Such searches can be performed using the NBLAST and XBLAST programs (version 2.0) of Altschul, et al. (J. Mol. Biol. 215:403-10 (1990)). BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (Nucleic Acids Res. 25(17):3389-3402 (1997)). When utilizing BLAST and gapped BLAST programs, the default parameters of the respective programs (e.g., XBLAST and NBLAST) can be used.

In one embodiment, a “homolog” of a specifically named nucleotide sequence refers to an oligonucleotide sequence in which one or more triplets are replaced with different triplets that encode the same amino acid as governed by the redundancy of the genetic code (i.e., 61 different DNA triplets, which correspond to RNA codons, are known to encode 20 different amino acids). Such homologs may be desirable to exploit the bias in codon usage by different species thereby ensuring optimal translational efficiency of the nucleotide sequence.

The following summarizes the DNA triplets encoding different amino acids: phenylalanine is encoded by TTT, TTC, leucine is encoded by TTA, TTG, CTT, CTC, CTA, CTG, isoleucine is encoded by ATT, ATC, ATA, methionine is encoded by ATG, valine is encoded by GTT, GTC, GTA, GTG, serine is encoded by TCT, TCC, TCA, TCG, proline is encoded by CCT, CCC, CCA, CCG, threonine is encoded by ACT, ACC, ACA, ACG, alanine is encoded by GCT GCC, GCA, GCG, tyrosine is encoded by TAT, TAC, histidine is encoded by CAT, CAC, glutamine is encoded by CAA, CAG, asparagine is encoded by AAT, AAC, lysine is encoded by AAA, AAG, aspartic acid is encoded by GAT, GAC, glutamic acid is encoded by GAA, GAG, cysteine is encoded by TGT, TGC, tryptophan is encoded by TGG, arginine is encoded by CGT, CGC, CGA, CGG, serine is encoded by AGT, AGC, arginine is encoded by AGA, AGG, glycine is encoded by GGT, GGC, GGA, GGG, stop codons include TAA, TAG, TGA.

For example, homologs of the DNA sequence encoding ADF are illustrated by, but not limited to, the corresponding portion of the nucleotide sequence SEQ ID NO:5 (GenGenBank: NP_{—}005909) in which the triplet encoding the following amino acids of the corresponding amino acid sequence SEQ ID NO:7 is as follows: phenylalanine at one or more of amino acid positions 20,22,39,50,73,95 is encoded independently by any one of TTT, TTC, leucine at one or more of amino acid positions 11,24,54,55,56,65,85,99 is encoded independently by any one of TTA, TTG, CTT, CTC, CTA, CTG, isoleucine at one or more of amino acid positions 61,67,78,82,89 is encoded independently by any one of ATT, ATC, ATA, valine at one or more of amino acid positions 21,25,34,35,40,70,96 is encoded independently by any one of GTT, GTC, GTA, GTG, serine at one or more of amino acid positions 8,12,23,38,42,51,71,72,79,88 is encoded independently by any one of TCT, TCC, TCA, TCG, proline at one or more of amino acid positions 53,83 is encoded independently by any one of CCT, CCC, CCA, CCG, threonine at one or more of amino acid positions 10,45,48,52,98 is encoded independently by any one of ACT, ACC, ACA, ACG, alanine at one or more of amino acid positions 2, 4, 6, 9, 14, 16, 18, 27, 81, 87 is encoded independently by any one of GCT GCC, GCA, GCG, tyrosine at one or more of amino acid positions 15, 49 is encoded independently by any one of TAT, TAC, glutamine at amino acid position 43 is encoded independently by any one of CAA, CAG, asparagine at one or more of amino acid positions 29, 64 is encoded independently by any one of AAT, AAC, lysine at one or more of amino acid positions 1, 3, 31, 41, 58, 59, 63, 69, 76, 86, 90, 91, 97, 100 is encoded independently by any one of AAA, AAG, aspartic acid at one or more of amino acid position 26,80,94 is encoded independently by any one of GAT, GAC, glutamic acid at one or more of amino acid positions 32, 36, 44, 46, 62, 74,75, 84, 93 is encoded independently by any one of GAA, GAG, cysteine at one or more of amino acid positions 37,47 is encoded independently by any one of TGT, TGC, arginine at amino acid position 19 is encoded independently by any one of CGT, CGC, CGA, CGG, serine at one or more of amino acid positions 8, 12, 23, 38, 42, 51, 71, 72, 79, 88 is encoded independently by any one of AGT, AGC, and glycine at one or more of amino acid positions 5, 7, 17, 30, 33, 57, 60, 66, 68, 92 is encoded independently by any one of GGT, GGC, GGA, GGG.

In another embodiment, a “homolog” of a specifically named nucleotide sequence refers to an oligonucleotide sequence in which one or more codons are replaced with different codons that encode a different amino acid. For example, it may be desirable to use a homolog which exhibits greater than 50% identity to the specifically named nucleotide sequence.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR, or the enzymatic cleavage of a polynucleotide by a ribozyme.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution of 5×SSPE (43.8 g/l NaCl, 6.9 g/l NaH2PO4—H2O and 1.85 g/l EDTA, pH adjusted to 7.4 with NaOH), 0.5% SDS, 5× Denhardt's reagent and 100 ìg/ml denatured salmon sperm DNA followed by washing in a solution comprising 0.1× SSPE, 1.0% SDS at 42° C. when a probe of about 500 nucleotides in length is employed. In another embodiment, high stringency conditions comprise conditions equivalent to binding or hybridization at 68° C. in a solution containing 5×SSPE, 1% SDS, 5× Denhardt's reagent and 100 μg/ml denatured salmon sperm DNA followed by washing in a solution containing 0.1×SSPE, and 0.1% SDS at 68° C.

“Medium stringency conditions” when used in reference to nucleic acid hybridization comprise conditions equivalent to binding or hybridization at 42° C. in a solution of 5×SSPE (43.8 g/l NaCl, 6.9 g/l NaH2PO4—H2O and 1.85 g/l EDTA, pH adjusted to 7.4 with NaOH), 0.5% SDS, 5× Denhardt's reagent and 100 ìg/ml denatured salmon sperm DNA followed by washing in a solution comprising 1.0×SSPE, 1.0% SDS at 42° C.

“Low stringency conditions” comprise conditions equivalent to binding or hybridization at 42° C. in a solution of 5×SSPE (43.8 g/l NaCl, 6.9 g/l NaH2PO₄.H2O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharamcia), 5 g BSA (Fraction V; Sigma)) and 100 Fg/ml denatured salmon sperm DNA followed by washing in a solution comprising 5×SSPE, 0.1% SDS at 42° C.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 50% to 70% homology to the first nucleic acid sequence, then another hybridization condition is said to be equivalent to the hybridization condition of interest if this other hybridization condition also results in hybridization of the first nucleic acid sequence with the other nucleic acid sequences that have from 50% to 70% homology to the first nucleic acid sequence.

Thus, with reference to the amino acid sequences of the invention, such as SEQ ID NO:1 (GenBank: P00346), SEQ ID NO:2 (GenBank: P00346), SEQ ID NO:3 (GenBank: P00346), SEQ ID NO:4 (GenBank: NP_{—}005909), SEQ ID NO:6 (GenBank: NP_{—}005909), SEQ ID NO:7 (GenBank: NP_{—}005909), SEQ ID NO:8 (GenGenBank: NM_{—}145074), SEQ ID NO:10 (GenBank: AF100928), SEQ ID NO:12 (GenBank: AF298770), SEQ ID NO:14 (GenBank: BC026033), SEQ ID NO:16 (Bcl-2 GenBank: M14745), SEQ ID NO:18 (GenBank: NM_{—}138764), SEQ ID NO:20 (GenBank:AF031523), SEQ ID NO:22 (GenBank: AF250233), SEQ ID NO:24 (GenBank: NM_{—}004435), SEQ ID NO:26 (GenBank: AB013918), SEQ ID NO:28 (GenBank: BC007112), SEQ ID NO:30 (GenBank: NM_{—}001963), SEQ ID NO:32 (GenBank: AY047581), SEQ ID NO:34 (GenBank: NM_{—}001885), SEQ ID NO:36 (GenBank: M20704), SEQ ID NO:38 (GenBank: BT006856), SEQ ID NO:40 (GenBank: AJ298844), SEQ ID NO:42 (GenBank: AF060222), SEQ ID NO:44 (GenBank: U10421), SEQ ID NO:46 (GenBank: NM_{—}000612), SEQ ID NO:48 (GenBank: NM_{—}033137), SEQ ID NO:50 (GenBank: AY463230), SEQ ID NO:72 (GenBank: AY339584), SEQ ID NO:74 (GenBank: AF452712), SEQ ID NO:76 (GenBank: AF002697), and SEQ ID NO:78 (GenBank: AF544398), equivalent amino acid sequences within the scope of the invention include sequences that hybridize preferentially to these sequences or to portions thereof, at 60° C. at 0.5 M salt, and more preferentially at 60° C. at 0.75 M or 1 M salt, and yet more preferably at 60° C. at 1.5 M salt.

Additional equivalent sequences that hybridize under different levels of hybridization stringency may be isolated using known conditions that increase stringency of a hybridization reaction, such as (in order of increasing stringency): incubation temperatures of 25 degrees C., 37 degrees C., 50 degrees C. and 68 degrees C.; buffer concentrations of 10×SSC, 6×SSC, 1×SSC, 0.1×SSC (where 1×SSC is 0.15 M NaCl and 15 mM citrate buffer) and their equivalents using other buffer systems; formamide concentrations of 0%, 25%, 50%, and 75%; incubation times from 24 hours to 5 minutes; 1, 2, or more washing steps; wash incubation times of 1, 2, or 15 minutes; and wash solutions of 6×SSC, 1×SSC, 0.1×SSC, or deionized water.

Thus, in one embodiment, the term “nucleic acid sequence encoding mitochondrial malate dehydrogenase” includes sequences that encode the exemplary human (SEQ ID NO:4) (GenGenBank: NP_{—}005909) and/or pig (SEQ ID NO:1) (GenBank: P00346), fragments thereof and variants thereof. Such nucleic acid sequences are exemplified by the human SEQ ID NO:5 (GenGenBank: NP_{—}005909) and to equivalent fragments thereof, homologs thereof, and sequences that hybridize under highly stringent and/or medium stringent conditions to SEQ ID NO:5 and/or to its portions.

In another embodiment, the term “nucleic acid sequence encoding minimum activator of DNA fragmentation” refers to a nucleic acid sequence that encodes human (SEQ ID NO:6) (GenGenBank: NP_{—}005909), pig (SEQ ID NO:2) (GenBank: P00346), fragments thereof, and/or variants thereof. Such nucleic acid sequences are exemplified by portions of SEQ ID NO5:, and to equivalent fragments thereof, homologs thereof, and sequences that hybridize under highly stringent and/or medium stringent conditions to SEQ ID NO:5 and/or to its portions.

In a further embodiment, the term “nucleic acid sequence encoding activator of DNA fragmentation” refers to a nucleic acid sequence that encodes human (SEQ ID NO:7) (GenGenBank: NP_{—}005909), pig (SEQ ID NO:3) (GenBank: P00346), fragments thereof, and/or variants thereof. Such nucleic acid sequences are exemplified by portions of SEQ ID NO5:, and to equivalent fragments thereof, homologs thereof, and sequences that hybridize under highly stringent and/or medium stringent conditions to SEQ ID NO:5 and/or to its portions.

In yet another embodiment, the term “nucleic acid sequence encoding” when made in reference to nucleic acid sequences that encode the exemplary proteins Htra/Omi, apoptosis inducing factor, Smac/DIABLO, EndoG, Cytochrome C, Nix, Nip3, CIDE-B, gelsolin, Bcl-2, Bax, Bad, Bid, caspase-activated DNase, DNase I, DNase II, inhibitor of CAD nuclease, epidermal growth factor, vascular endothelial growth factor, lens crystalline protein, antennapedia protein, fibronectin type 1, human HOX protein, insulin-like growth factor, and fibroblast growth factor, refers to the exemplary nucleic sequences SEQ ID NOs:9, 11, 13, 25, 73, 75, 77, 79, 15, 17, 19, 21, 23, 27, 41, 43, 29, 31, 33, 35, 37, 39, 45, 47, and 49, respectively, and to equivalent fragments thereof, homologs thereof, and sequences that hybridize under highly stringent and/or medium stringent conditions to these nucleotide sequence and/or to their portions.

In one embodiment, the portion of MDH that is encoded by the invention nucleotide sequences comprises one or more MADF sequence, and more preferably comprises one or more ADF sequence. In a preferred embodiment, the encoded amino acid sequence has activity chosen from one or more of DNA nuclease activity and cell-killing activity. This may be desirable where expression of a protein that increases apopstosis is the goal, such as for use in the invention's methods. In a more preferred embodiment, the amino acid sequence having DNA nuclease activity and/or cell-killing activity further comprises one or more of antibody, ligand of a cell receptor, N-terminal signal peptide, cell internalization peptide, nuclear localization peptide, and a biotin binding protein (such as an anti-biotin antibody).

D. Vectors And Cells

The invention provides expression vectors that contain nucleic acid sequences encoding one or more portions of MDH. Preferably, the MDH protein contains MADF, and more preferable, the portion contains ADF. The invention also provides expression vectors that contain nucleic acid sequences encoding one or more of Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II, etc.

The invention's expression vectors are useful in the producing the invention's proteins, whose utility is further described herein.

As used herein, the terms “vector” and “vehicle” are used interchangeably in reference to nucleic acid molecules that transfer DNA segment(s) from one cell to another. Vectors are exemplified by, but not limited to, plasmids, linear DNA, encapsidated virus, etc. The term “expression vector” as used herein refers to a recombinant DNA molecule containing a desired coding sequence and appropriate nucleic acid sequences necessary for the expression (i.e., transcription and/or translation) of the operably linked coding sequence in a particular host organism. Expression vectors are exemplified by, but not limited to, plasmid, phagemid, shuttle vector, cosmid, virus, chromosome, mitochondrial DNA, plastid DNA, and nucleic acid fragment. Nucleic acid sequences used for expression in prokaryotes include a promoter, optionally an operator sequence, a ribosome binding site and possibly other sequences. Eukaryotic cells are known to utilize promoters, enhancers, and termination and polyadenylation signals.

While not required, in one embodiment, the amino acid sequence encoded by the expression vector has activity chosen from one or more of DNA nuclease activity and cell-killing activity. In another embodiment, the encoded amino acid further comprises one or more of antibody, ligand of a cell receptor, N-terminal signal peptide, cell internalization peptide, nuclear localization peptide, radionuclide, and a biotin binding protein (such as an anti-biotin antibody.

Methods for making the invention's expression vectors are known in the art. For example, in the cloning and expression of the exemplary ADF protein, the following steps may be performed: (1) Isolation and purification of the messenger RNA (mRNA) for ADF protein, (2) Conversion of the mRNA into double-stranded DNA (ds-cDNA), (3) Construction of ds-cDNA having an oligo-dC tail added thereto, (4) Construction of a hybrid plasmid by joining the oligo-dC tailed ds-cDNA to a vector having an oligo-dG tail added thereto, (5) Transformation of a microorganism and selection of clones, (6) Confirmation of the characters of the ADF protein gene region by analysis of the DNA sequence, and (7) Confirmation of the expression of ADF protein enzyme activity.

The sequence encoding ADF (such as the portion of SEQ ID NO:5) can be incorporated into vectors capable of replicating in various hosts (such as Escherichia coli, Bacillus subtilis, bakers' yeast, etc.), by inserting it between the 3′-terminus of the promoter region functioning in the respective hosts and the 5′-terminus of the terminator region. Thus, there can be constructed recombinant DNA plasmids permitting the expression of ADF protein.

An expression vector may contain a promoter. The term “promoter” as used herein, refers to a nucleotide sequence which when ligated to a nucleotide sequence of interest is capable of controlling the transcription of the nucleotide sequence of interest into mRNA.

The promoter region may additionally contain a translation initiation region. For example, where the host is E. coli, the translation initiation region extends from the Shine-Dalgarno sequence or the ribosome binding site (i.e., the site corresponding to the nucleotide sequence of mRNA to which a ribosome can bind) to the initiator codon (e.g., ATG). Preferably, the distance between the Shine-Dargarno sequence and the initiator codon is about 10 bases long.

Where the host is a prokaryote such as E. coli, the terminator region is not always necessary. However, the presence of a terminator region is known to have some additional effects. Accordingly, where E. coli is used as the host, the structural gene for ADF protein may be inserted into a plasmid capable of replicating in E. coli, at the 3′-terminus of the promoter region present in the plasmid and functioning in E. coli. Preferred promoter regions include, for example, the tryptophan (trp) promoter, the lactose (lac) promoter, the tac promoter, the PL lambda promoter and the like. Thus, various vectors (such as pBR322, pUC and the like) containing these promoter regions are useful in the present invention.

In practice, such a vector is cleaved with a suitable restriction endonuclease at the 3′-terminus of the promoter region. If the structural gene for ADF protein has the same cohesive ends, it can be directly inserted into the vector. If the cohesive ends of the ADF protein gene have unmatched DNA sequences, flush ends are generated. Then, the ADF protein gene can be inserted into the vector by means of a ligase.

A recombinant DNA plasmid constructed by inserting the ADF protein gene between the 3′-terminus of the promoter region and the 5′-terminus of the terminator region can be used to transform E. coli according to well-known methods.

The invention's expression vectors may contain a “selectable marker-encoding nucleotide sequence,” which refers to a nucleotide sequence that is capable of expression in host cells and where expression of the selectable marker confers to cells containing the expressed gene the ability to grow in the presence of a corresponding selective agent. Selectable marker genes are exemplified by the bacterial aminoglycoside 3′ phosphotransferase gene (also referred to as the neo gene) which confers resistance to the drug G418 in cells, (2) the bacterial hygromycin G phosphotransferase (hyg) gene which confers resistance to the antibiotic hygromycin, and (3) the bacterial xanthine-guanine phosphoribosyl transferase gene (also referred to as the gpt gene) which confers the ability to grow in the presence of mycophenolic acid.

The invention also provides host cells that contain the invention's expression vectors. A “host cell” includes an individual cell or cell culture which can be or has been a recipient for vector(s) or for incorporation of nucleic acid molecules and/or proteins. Host cells include progeny of a single host cell, and the progeny may not necessarily be completely identical (in morphology or in genomic of total DNA complement) to the original parent cell due to natural, accidental, or deliberate mutation. A host cell includes cells transfected in vivo with a polynucleotide(s) of this invention.

A host cell may be from a cell line or cell culture. A “cell line” or “cell culture” denotes cells grown or maintained in vitro. It is understood that the descendants of a cell may not be completely identical (either morphologically, genotypically, or phenotypically) to the parent cell. Cells described as “uncultured” are obtained directly from a living organism, and are generally maintained for a limited amount of time away from the organism (i.e., not long enough or under conditions for the cells to undergo substantial replication).

Host cells that contain the invention's vectors include, without limitation, prokaryotic cells, such as E. coli, or eukaryotic cells such as yeast, plant, insect, amphibian, or an animal (such as B cell, stromal cell of lymph organ such as spleen, fibroblast cell such as embryo fibroblasts (EFs), including mouse embryo fibroblasts (MEFs), macrophage cell such as stromal macrophage cell, dendritic cell, neuron cell, plasma cell, lymphoid cell, lymphoblastoid cell, myeloid cell, Reed-Sternber (HRS) cell of Hodgkin's lymphomas, epithelial cell such as breast cell, gastric cell, lung cell, prostate cell, cervical cell, pancreatic cell, colon cell, rectal cell, ovarian cell, stomach cell, esophagus cell, mouth cell, tongue cell, gum cell, skin cell, muscle cell, heart cell, liver cell, bronchial cell, cartilage cell, bone cell, testis cell, kidney cell, endometrium cell, uterus cell, bladder cell, gastrointestinal tract cell, thyroid cell, brain cell, gall bladder cell, gastrointestinal tract cell, and ocular cell (such as cell of the cornea, cell of uvea, cell of the choroids, cell of the macula, vitreous humor cell, etc.). An “animal” as used herein refers to any multicellular animal, including mammals (e.g., humans, non-human primates, rodents such as mouse, rat and guinea pig, ovines, bovines, ruminants, lagomorphs, porcines, caprines, equines, canines, felines, aves, etc.).

The expression vectors may be introduced into a cell, and the resulting transformants can be selected on the basis of a phenotypic character such as drug resistance (e.g., resistance to ampicillin), auxotrophy or the like. Then, cells having ADF protein activity are selected from the cells exhibiting such a phenotypic character. A transformant selected in the above-described manner can be grown using well-known methods. The medium used for this purpose can be, for example, a broth or a synthetic medium containing glucose and/or other required nutrient(s).

If it is desired to cause the promoter to function more efficiently, a chemical agent such as isopropyl-β-thiogalactoside (hereinafter abbreviated as IPTG) or indoleacrylic acid (hereinafter abbreviated as IAA) may be added to the medium.

The transformant is usually incubated at a temperature of 15 to 43° C., preferably 28 to 42° C., for a period of 4 to 48 hours, preferably 4 to 20 hours. If necessary, aeration and/or agitation may be employed.

Where bakers' yeast (Saccharomyces cerevisiae) is used as the host, its transformants can be created in the following manner: Into an E. coli-yeast shuttle vector, such as YRp7 or pMA3 as previously described, is inserted a promoter region functioning in bakers' yeast, such as the promoter region of the glyceraldehyde-3-phosphate dehydrogenase gene, or the promoter region of the alcohol dehydrogenase I gene. Then, a DNA fragment containing the structural gene for ADF protein is joined to the 3′-terminus of the inserted promoter region by means of a ligase. Further, the 3′-terminal untranslated region, which is not translated by mRNA and which is included in the alcohol dehydrogenase I gene, or the 3′-terminal untranslated region which is not translated by mRNA and which is included in the glyceraldehyde-3-phosphate dehydrogenase gene as a terminator is selected and joined to the 3′-terminus of the structural gene for ADF by means of a ligase. Thereafter, the plasmid is cyclized by joining the 3′-terminus of the selected untranslated region to the 5′-terminus of the shuttle vector.

Using this cyclized plasmid, E. coli is transformed according to well-known methods. The resulting transformants can be selected on the basis of a phenotypic character such as resistance to ampicillin. From the cells of these E. coli transformants, plasmid DNA is isolated according to the alkali extraction method. Using this plasmid, an auxotrophic strain of yeast (such as MT-40391 lysine-dependent strain obtained by mutation of S. cerevisiae ATCC 44771 strain) is transformed according to well-known methods. The transformed yeast can be selected on the basis of reversion of the auxotrophy of the host. The transformed yeast can be grown in any of various well-known media. The medium used for this purpose can be, for example, a medium prepared by adding glucose and other required nutrient(s) to Wickerham's amino acid-free medium.

The yeast is usually incubated at a temperature of 15 to 40° C. for a period of 24 to 72 hours. If necessary, aeration and/or agitation may be employed.

After the microorganism is grown in the above-described manner, the cells can be collected from the resulting culture according to any conventional procedure, and the ADF protein produced and accumulated in the collected cells can be extracted by destroying their cell wall and other cellular structures. To this end, there may be employed contact with an organic solvent, a surface active agent or the like; mechanical treatments such as sonication, glass bead disintegration and the like; and biochemical procedures such as treatment with a suitable lytic enzyme, autolysis and the like.

The crude enzyme prepared in the above-described manner, the immobilized cells obtained by embedding the collected cells in an immobilizing agent such as polyacrylamide gel or arginate gel, or the collected cells themselves may be used to effect the enzymatic reaction of an ammonia donor with cinnamic acid and thereby produce L-Phe. This enzymatic reaction can be carried out according to various conventional processes including, for example, the process of Japanese Patent Publication No. 44474/'86 in which the reaction mixture contains an ammonia donor in large excess relative to cinnamic acid and the concentration of cinnamic acid does not exceed the inhibitory level for the enzymatic reaction.

E. Antibodies Specific for ADF

The invention provides antibodies that specifically bind to a MDH portion. In one embodiment, the MDH portion comprises one or more of MADF, and more preferably comprises one or more of ADF. These antibodies are useful in, for example, detecting the presence of ADF in the cell cytoplasm, thereby identifying the cells as apoptotic, and/or quantitating the degree of apoptosis in the cells, as further described below. The invention's antibodies are also useful for identifying test agents that reduce cell apoptosis, as further described below. In a preferred embodiment, binding of the antibody to the MDH portion (such as MADF and ADF portions) reduces one or more of DNA nuclease activity and cell-killing activity of the MDH portion.

The invention expressly contemplates that the antibody that specifically binds to a MDH portion (such as MADF and ADF portions) is monoclonal, polyclonal, chimeric, de-immunized, and/or humanized, including phage display derived antibody fragments such as Fab and scFV. In a preferred embodiment, the invention's antibodies specifically bind to uncleaved mitochondrial malate dehydrogenase (MDH) with a reduced affinity than to ADF.

F. Methods For Killing Cells

The invention provides methods for killing cells, comprising: a) providing: i) cells; and ii) an amino acid sequence comprising or more of MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II; and b) contacting the cells with the amino acid sequence to produce contacted cells wherein cell death (such as by apoptosis) of the contacted cells is increased.

In another embodiment, the invention provides a method for killing cells, comprising: a) providing: i) a cell comprising a cell marker molecule; ii) a first composition comprising an antibody that specifically binds to biotin operably linked to a first molecule that specifically binds to the cell marker molecule; and iii) a second composition comprising biotin operably linked to a second molecule chosen from one or more of MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II; b) contacting the first composition with the cells such that the first molecule of the first composition specifically binds to the cell marker molecule to produce a contacted cell; and c) contacting the second composition with the contacted cell such that the antibody of the first composition specifically binds to the biotin of the second composition, thereby increasing cell death (such as by apoptosis) of the contacted cell. Seps b) and c) may be simultaneous, or sequential in any order, i.e., in the order b) followed by c), or c) followed by b). In a preferred embodiment, the first composition further comprises a cell internalization peptide operably linked to the first molecule. In another preferred embodiment, the second composition further comprises a cell internalization peptide operably linked to the second molecule.

The invention's methods are useful in, for example, reducing symptoms of diseases (e.g. cancer) that are associated with undesirable cell proliferation. Additional uses are in determining the role a cell (such as endothelial cell) plays in development of a biological or clinical phenomenon (such as cancer) by determining whether cell death (such as by apoptosis) of that cell alters (i.e. increases or decreases) the phenomenon.

In one embodiment, the step of contacting the cells with the invention's compositions may be accomplished by mixing the amino acid sequence with the cells. In the case of an animal, contacting is performed by administering (such as by intravenous, intramuscular, subcutaneous, intraperitoneal, intranasal, topical, and sublingual, routes) the amino acid to the animal. In another embodiment, an expression vector encoding the invention's proteins is introduced into the cells to bring about expression of the proteins. In a particularly preferred embodiment, the invention amino acid sequences are conjugated with an antibody that targets the sequence to a cell of interest (such as a cancer cell). In a more preferred embodiment, these conjugates further contain one or more cell internalization peptides.

Generally, the increase in the level of cell death (such as by apoptosis) is determined with reference to a control. The term “control” as used herein when in reference to a sample, cell, tissue, animal, etc., refers to any type of sample, cell, tissue, animal, etc. that one of ordinary skill in the art may use for checking the results of another sample, cell, tissue, animal, etc., by maintaining the same conditions except in some one particular factor, and thus inferring the causal significance of this varied factor.

In one embodiment, the method further comprises detecting increased cell death (such as by apoptosis) in the contacted cells. It should be noted that the step of detecting increased cell death (such as apoptosis) is optional. This may be desirable where an animal is treated with the invention's compositions, and the end-point to be determine is downstream from cell death (such as by apoptosis), such as reduced disease symptoms in the treated animal.

In one embodiment the cells are in vitro. Using cells in vitro is useful in, for example, determining the efficacy of the invention compositions on DNA nuclease activity and/or cell-killing activity in the cell.

In an alternative embodiment, the cells are in vivo, such as in a mammalian animal. Such application is useful in animal models, clinical studies, and therapeutic interventions that employ the invention's compositions in diseases associated with undesirable increased cell proliferation. In one embodiment, the mammalian animal is chosen from one or more of an animal that has a disease and that is suspected of being capable of developing a disease, wherein the disease is associated with increased cell proliferation. Exemplary diseases that are associated with undesirable cell proliferation include, without limitation, one or more of angiogenesis, restenosis, atherosclerosis, cancer, tumor metastasis, fibrosis, hemangioma, lymphoma, leukemia, psoriasis, arthritis, autoimmune disease, diabetes, amyotrophic lateral sclerosis, graft rejection, retinopathy, macular degeneration, autoimmune disease (such as Lupus, Crohn's disease, and multiple sclerosis), and retinal tearing. Several target tissues and cells are amenable to administration of the invention's compositions. For example, in fibrosis the tissue includes heart, lung, and liver, in angiogenesis the cells include endothelial cells and vascular smooth muscle cells, in restenosis the cells include vascular smooth muscle cells, in atherosclerosis the cells include vascular smooth muscle cells, monocyte cells and macrophage cells, in hemangioma the cells include endothelial cells, in lymphoma and leukemia the cells include leukocyte cells, hematopoietic cells, and B cells, in psoriasis the cells include endothelial cells, in arthritis the cells include endothelial cells, synoviocyte cells, and fibroblast cells, in amyotrophic lateral sclerosis the cells include B cells, in graft rejection the cells include leukocyte cells, hematopoietic cells, and B cells, in allergy the cells include allergen specific antibody secreting cells, in retinopathy, macular degeneration, and retinal tearing, the cells include endothelial cells, in rheumatoid arthritis and osteoarthritis, the cells include bone cells and synovial cells, in psoriasis and skin cancer, the cells include skin cells.

In another embodiment, the mammalian animal to which the invention's compositions are administered has, or is suspected of being capable of developing a tumor. The terms “tumor” and “neoplasm” refer to a tissue growth which is characterized, in part, by increased cell proliferation.

Tumors may be benign and are exemplified, but not limited to, a hemangioma, glioma, teratoma, and the like. Tumors may alternatively be malignant. The terms “malignant neoplasm” and “malignant tumor” refer to a tumor which contains at least one cancer cell. A “cancer cell” refers to a cell undergoing early, intermediate or advanced stages of multi-step neoplastic progression as previously described (H. C. Pitot (1978) in “Fundamentals of Oncology,” Marcel Dekker (Ed.), New York pp 15-28). a cancer cell includes preneoplastic cells (such as hyperplastic cells and dysplastic cells) as well as neoplastic cells, that may be invasive. Thus, the term “cancer” is used herein to refer to a malignant tumor, which may or may not be metastatic. Malignant tumors that may benefit from administration using the invention's compositions include, for example, carcinomas such as lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant tumors are further exemplified by sarcomas (such as osteosarcoma and Kaposi's sarcoma). The invention expressly contemplates within its scope any malignant tumor. Preferably, the malignant tumor exhibits increased cell proliferation.

In one embodiment, administration of the invention compositions to the mammalian animal results in a reduction of at least one symptom that is associated with the disease (such as cancer).

The invention's compositions may be administered to the mammalian animal by any route, such as by intravenous, intramuscular, subcutaneous, intraperitoneal, intranasal, topical, and sublingual routes.

In one embodiment, where the disease is cancer, the invention's compositions are administered in combination with one or more of anti-cancer agent, radiation therapy, and protein-based therapy (such as antibody-based therapies using any one or more of 5-Fluorouracil, Leucovorin, Tomudex, Mitomycin C, CPT-11, and 3-bromopyruvate).

The terms and “anti-cancer,” “anti-cancer chemotherapeutic,” “anti-neoplastic,” and “anti-neoplastic chemotherapeutic” when in reference to a compound refer to a compound which reduces (including retards and/or completely arrests) the rate of neoplastic progression. The term also refers to a compound which reduces the number of cancer cells in the absence of a change in the rate of neoplastic progression. Anti-neoplastic compounds may be naturally occurring as well as man-made. Exemplary anti-cancer agents are known in the art such as, without limitation, those described in Goodman and Gilman's “Pharmaceutical Basis of Therapeutics” ninth edition, Eds. Hardman et al., 1996. Representative examples of anti-cancer agents include taxanes (e.g., paclitaxel and docetaxel). Etanidazole, Nimorazole, perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds, nitroimidazole, 5-substituted-4-nitroimidazoles, SR-2508, 2H-isoindolediones (U.S. Pat. No. 4,494,547), chiral (((2-bromoethyl)-amino)methyl)-nitro-1H-imidazole-1-ethanol (U.S. Pat. No. 5,543,527; U.S. Pat. No. 4,797,397; U.S. Pat. No. 5,342,959), nitroaniline derivatives (U.S. Pat. No. 5,571,845), DNA-affinic hypoxia selective cytotoxins (U.S. Pat. No. 5,602,142), halogenated DNA ligand (U.S. Pat. No. 5,641,764), 1,2,4 benzotriazine oxides (U.S. Pat. No. 5,616,584; U.S. Pat. No. 5,624,925; U.S. Pat. No. 5,175,287), nitric oxide (U.S. Pat. No. 5,650,442), 2-nitroimidazole derivatives (U.S. Pat. No. 4,797,397; U.S. Pat. No. 5,270,330; U.S. Pat. No. 5,270,330; Patent EP 0 513 351 B1), fluorine-containing nitroazole derivatives (U.S. Pat. No. 4,927,941), copper (U.S. Pat. No. 5,100,885), combination modality cancer therapy (U.S. Pat. No. 4,681,091), 5-CldC or (d)H.sub.4U an/or 5-halo-2′-halo-2′-deoxy-cytidine and/or -uridine derivatives (U.S. Pat. No. 4,894,364), platinum complexes (U.S. Pat. No. 4,921,963; Patent EP 0 287 317 A3), fluorine-containing nitroazole (U.S. Pat. No. 4,927,941), benzamide, autobiotics (U.S. Pat. No. 5,147,652), benzamide and nicotinamide (U.S. Pat. No. 5,215,738), acridine-intercalator (U.S. Pat. No. 5,294,715), fluorine-containing nitroimidazole (U.S. Pat. No. 5,304,654, Apr. 19, 1994), hydroxylated texaphyrins (U.S. Pat. No. 5,457,183), hydroxylated compound derivative (Publication Number 011106775 A (Japan), Oct. 22, 1987; Publication Number 01139596 A (Japan), Nov. 25, 1987; Publication Number 63170375 A (Japan)), fluorine containing 3-nitro-1,2,4-triazole (Publication Number 02076861 A (Japan), Mar. 31, 1988), 5-thiotretrazole derivative or its salt (Publication Number 61010511 A (Japan), Jun. 26, 1984), Nitrothiazole (Publication Number 61167616 A (Japan) Jan. 22, 1985), imidazole derivatives (Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (Publication Number 62039525 A (Japan), Aug. 15, 1985), 3-nitro-1,2,4-triazole (Publication Number 62138427 A (Japan), Dec. 12, 1985), Carcinostatic action regulator (Publication Number 63099017 A (Japan), Nov. 21, 1986), 4,5-dinitroimidazole derivative (Publication Number 63310873 A (Japan) Jun. 9, 1987), nitrotriazole Compound (Publication Number 07149737 A (Japan) Jun. 22, 1993), cisplatin, doxorubin, misonidazole, mitomycin, tiripazamine, nitrosourea, mercaptopurine, methotrexate, flurouracil, bleomycin, vincristine, carboplatin, epirubicin, doxorubicin, cyclophosphamide, vindesine, etoposide (Tannock. Journal of Clinical Oncology 14(12):3156-3174, 1996), camptothecin (Ewend et al. Cancer Research 56(22):5217-5223, 1996) and paclitaxel (Tishler et al. Journal of Radiation Oncology and Biological Physics 22(3):613-617, 1992).

A number of the above-mentioned chemotherapeutic agents also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA).sub.2Pt(DOLYM) and (DACH)Pt(DOLYM) cisplatin, Cis-(PtCl.sub.2(4,7-H-5-methyl-7-oxo-) 1,2,4(triazolo(1,5-a)pyrimidine).sub.2), (Pt(cis-1,4-DACH)(trans-Cl.sub.2)(CBDCA)).multidot.-1/2MeOH cisplatin, 4-pyridoxate diammine hydroxy platinum, Pt(II).Pt(II) (Pt.sub.2(NHCHN(C(CH.sub.2)(CH.s-ub.3))).sub.4), 254-S cisplatin analogue, O-phenylenediamine ligand bearing cisplatin analogues, trans, cis-(Pt(OAc).sub.2I.sub.2(en)), estrogenic 1,2-diarylethylenediamine ligand (with sulfur-containing amino acids and glutathione) bearing cisplatin analogues, cis-1,4-diaminocyclohexane cisplatin analogues, 5′ orientational isomer of cis-(Pt(NH.sub.3)(4-aminoTEMP-O){d(GpG)}), chelating diamine-bearing cisplatin analogues, 1,2-diarylethyleneamine ligand-bearing cisplatin analogues, (ethylenediamine)platinum-(II) complexes, CI-973 cisplatin analogue, cis-diamminedichloroplatinum(II) and its analogues cis-1,1-cyclobutanedicarbosylato(2R)-2-methyl-1,4-butanediam-mineplatinum-(II) and cis-diammine(glycolato)platinum, cis-amine-cyclohexylamine-dichloroplatinum(II), gem-diphosphonate cisplatin analogues (FR 2683529), (meso-1,2-bis(2,6-dichloro-4-hydroxyplenyl)ethylenediamine) dichloroplatinum(II), cisplatin analogues containing a tethered dansyl group, platinum(II) polyamines, cis-(3H)dichloro(ethylenediamine)platinu-m(II), trans-diamminedichloroplatinum(II) and cis-(Pt(NH.sub.3).sub.2(N.sub.3-cy-tosine)Cl), 3H-cis-1,2-diaminocyclohexanedichloroplatinum(II) and 3H-cis-1,2-diaminocyclohexane-malonatoplatinum (II), diaminocarboxylatoplatinum (EPA 296321), trans-(D,1)-1,2-diaminocyclohexa-ne carrier ligand-bearing platinum analogues, aminoalkylaminoanthraquinone-deri-ved cisplatin analogues, spiroplatin, carboplatin, iproplatin and JM40 platinum analogues, bidentate tertiary diamine-containing cisplatinum derivatives, platinum(II), platinum(IV), cis-diammine (1,1-cyclobutanedicarboxylato-)platinum(II) (carboplatin, JM8) and ethylenediammine-malonatoplatinum(II) (JM40), JM8 and JM9 cisplatin analogues, (NPr4)2((PtCL4).cis-(PtC12-(NH2Me)2)), aliphatic tricarboxylic acid platinum complexes (EPA 185225), cis-dichloro(amino acid)(tert-butylamine)platinum-(II) complexes; 4-hydroperoxycylcophosphamide, acyclouridine cyclophosphamide derivatives, 1,3,2-dioxa- and -oxazaphosphorinane cyclophosphamide analogues, C5-substituted cyclophosphamide analogues, tetrahydrooxazine cyclophosphamide analogues, phenyl ketone cyclophosphamide analogues, phenylketophosphamide cyclophosphamide analogues, ASTA Z-7557 cyclophosphamide analogues, 3-(1-oxy-2,2,6,6-tetramethyl-4-piperidinyl)cy-clophosphamide, 2-oxobis(2-β-chloroethylamino)-4-,6-dimethyl-1,3,2-oxazaphosphorinan-e cyclophosphamide, 5-fluoro- and 5-chlorocyclophosphamide, cis- and trans-4-phenylcyclophosphamide, 5-bromocyclophosphamide, 3,5-dehydrocyclophosphamide, 4-ethoxycarbonyl cyclophosphamide analogues, arylaminotetrahydro-2H-1,3,2-oxazaphosphorine 2-oxide cyclophosphamide analogues, NSC-26271 cyclophosphamide analogues, benzo annulated cyclophosphamide analogues, 6-trifluoromethylcyclophosphamide, 4-methylcyclophosphamide and 6-methycyclophosphamide analogues; FCE 23762 doxorubicin derivative, annamycin, ruboxyl, anthracycline disaccharide doxorubicin analogue, N-(trifluoroacetyl)doxorubicin and 4′-O-acetyl-N-(trifluoroacetyl)-doxorubicin, 2-pyrrolinodoxorubicin, disaccharide doxorubicin analogues, 4-demethoxy-7-O-(2,6-dideoxy-4-O-(2,3,6-trideoxy-3-amino-α-L-lyxo-h-exopyranosyl)-α-L-lyxo-hexopyranosyl) adriamicinone doxorubicin disaccharide analog, 2-pyrrolinodoxorubicin, morpholinyl doxorubicin analogues, enaminomalonyl-β-alanine doxorubicin derivatives, cephalosporin doxorubicin derivatives, hydroxyrubicin, methoxymorpholino doxorubicin derivative, (6-maleimidocaproyl)hydrazone doxorubicin derivative, N-(5,5-diacetoxypent-1-yl) doxorubicin, FCE 23762 methoxymorpholinyl doxorubicin derivative, N-hydroxysuccinimide ester doxorubicin derivatives, polydeoxynucleotide doxorubicin derivatives, morpholinyl doxorubicin derivatives (EPA 434960), mitoxantrone doxorubicin analogue, AD198 doxorubicin analogue, 4-demethoxy-3′-N-trifluoroacetyldoxorubicin, 4′-epidoxorubicin, alkylating cyanomorpholino doxorubicin derivative, deoxydihydroiodooxorubicin (EPA 275966), adriblastin, 4′-deoxydoxorubicin, 4-demethyoxy-4′-o-methyldoxorubicin, 3′-deamino-3′-hydroxydoxorubicin, 4-demethyoxy doxorubicin analogues, N-L-leucyl doxorubicin derivatives, 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin, aglycone doxorubicin derivatives, SM 5887, MX-2,4′-deoxy-13(S)-dihydro-4′-iododoxorubicin (EP 275966), morpholinyl doxorubicin derivatives (EPA 434960), 3′-deamino-3′-(4-methoxy-1-piperidi-nyl) doxorubicin derivatives (U.S. Pat. No. 4,314,054), doxorubicin-14-valerate, morpholinodoxorubicin (U.S. Pat. No. 5,004,606), 3′-deamino-3′-(3′-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole, azo and azoxy misonidazole derivatives; RB90740; 6-bromo and 6-chloro-2,3-dihydro-1,4-benzothi-azines nitrosourea derivatives, diamino acid nitrosourea derivatives, amino acid nitrosourea derivatives, 3′,4′-didemethoxy-3′,4′-dio-xo-4-deoxypodophyllotoxin nitrosourea derivatives, ACNU, tertiary phosphine oxide nitrosourea derivatives, sulfamerizine and sulfamethizole nitrosourea derivatives, thymidine nitrosourea analogues, 1,3-bis(2-chloroethyl)—1-nitrosourea, 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine, pyrimidine (II) nitrosourea derivatives, CGP 6809, B-3839, 5-halogenocytosine nitrosourea derivatives, 1-(2-chloroethyl)-3-isobu-tyl-3-(β-maltosyl)-1-nitrosourea, sulfur-containing nitrosoureas, sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives, CNCC, RFCNU and chlorozotocin, CNUA, 1-(2-chloroethyl)-3-isobutyl-3—(β-maltosyl)-1-nitrosourea, choline-like nitrosoalkylureas, sucrose nitrosourea derivatives (JP 84219300), sulfa drug nitrosourea analogues, DONU, N,N′-bis (N-(2-chloroethyl)-N-nitrosocarbamoyl)cystamine (CNCC), dimethylnitrosourea, GANU, CCNU, 5-aminomethyl-2′-deoxyuridine nitrosourea analogues, TA-077, gentianose nitrosourea derivatives (JP 82 80396), CNCC, RFCNU, RPCNU AND chlorozotocin (CZT), thiocolchicine nitrosourea analogues, 2-chloroethyl-nitrosourea, ACNU, (1-(4-amino-2-methyl-5-pyrimidinyl)methyl-3-(2-chloroethyl)-3-nitrosourea hydrochloride), N-deacetylmethyl thiocolchicine nitrosourea analogues, pyridine and piperidine nitrosourea derivatives, methyl-CCNU, phensuzimide nitrosourea derivatives, ergoline nitrosourea derivatives, glucopyranose nitrosourea derivatives (JP 78 95917), 1-(2-chloroethyl)-3-cyclohexyl-1-nitrosourea, 4-(3-(2-chloroethyl)-3-nitrosoureid-o)-cis-cyclohexanecarboxylic acid, RPCNU (ICIG 1163), IOB-252, 1,3-bis(2-chloroethyl)-1-nitrosourea (BCNU), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives, 6-mercaptopurine (6-MP), 7,8-polymethyleneimidazo-1,3,2-diazaph-osphorines, azathioprine, methyl-D-glucopyranoside mercaptopurine derivatives and s-alkynyl mercaptopurine derivatives; indoline ring and a modified ornithine or glutamic acid-bearing methotrexate derivatives, alkyl-substituted benzene ring C bearing methotrexate derivatives, benzoxazine or benzothiazine moiety-bearing methotrexate derivatives, 10-deazaminopterin analogues, 5-deazaminopterin and 5,10-dideazaminopterin methotrexate analogues, indoline moiety-bearing methotrexate derivatives, lipophilic amide methotrexate derivatives, L-threo-(2S,4S)-4-fluoro-glutamic acid and DL-3,3-difluoroglutamic acid-containing methotrexate analogues, methotrexate tetrahydroquinazoline analogue, N-(ac-aminoacyl) methotrexate derivatives, biotin methotrexate derivatives, D-glutamic acid or D-erythrou, threo-4-fluoroglutamic acid methotrexate analogues, β,γ-methano methotrexate analogues, 10-deazaminopterin (10-EDAM) analogue, γ-tetrazole methotrexate analogue, N-(L-α-aminoacyl) methotrexate derivatives, meta and ortho isomers of aminopterin, hydroxymethylmethotrexate (DE 267495), γ-fluoromethotrexate, polyglutamyl methotrexate derivatives, gem-diphosphonate methotrexate analogues (WO 88/06158), α- and γ-substituted methotrexate analogues, 5-methyl-5-deaza methotrexate analogues (U.S. Pat. No. 4,725,687), N.delta.-acyl-N α-(4-amino-4-deoxypteroyl)-L-omithine derivatives, 8-deaza methotrexate analogues, acivicin methotrexate analogue, polymeric platinol methotrexate derivative, methotrexate-γ-dimyristoylphophatidylethanolamine, methotrexate polyglutamate analogues, poly-γ-glutamyl methotrexate derivatives, deoxyuridylate methotrexate derivatives, iodoacetyl lysine methotrexate analogue, 2,.omega.-diaminoalkanoid acid-containing methotrexate analogues, polyglutamate methotrexate derivatives, 5-methyl-S-deaza analogues, quinazoline methotrexate analogue, pyrazine methotrexate analogue, cysteic acid and homocysteic acid methotrexate analogues (U.S. Pat. No. 4,490,529), γ-tert-butyl methotrexate esters, fluorinated methotrexate analogues, folate methotrexate analogue, phosphonoglutamic acid analogues, poly (L-lysine) methotrexate conjugates, dilysine and trilysine methotrexate derivates, 7-hydroxymethotrexate, poly-y-glutamyl methotrexate analogues, 3′,5′-dichloromethotrexate, diazoketone and chloromethylketone methotrexate analogues, 10-propargylaminopterin and alkyl methotrexate homologs, lectin derivatives of methotrexate, polyglutamate methotrexate derivatives, halogentated methotrexate derivatives, 8-alkyl-7,8-dihydro analogues, 7-methyl methotrexate derivatives and dichloromethotrexate, lipophilic methotrexate derivatives and 3′,5′-dichloromethotrexate, deaza amethopterin analogues, MX068 and cysteic acid and homocysteic acid methotrexate analogues (EPA 0142220); N3-alkylated analogues of 5-fluorouracil, 5-fluorouracil derivatives with 1,4-oxaheteroepane moieties, S-fluorouracil and nucleoside analogues, cis- and trans-5-fluoro-5,6-dihydro-6-alkoxyuracil, cyclopentane 5-fluorouracil analogues, A-OT-fluorouracil, N4-trimethoxybenzoyl-5′-deoxy-5-fluoro-cytidine and 5′-deoxy-5-fluorouridine, 1-hexylcarbamoyl-5-fluorouracil, B-3839, uracil-1-(2-tetrahydrofuryl)-5-fluorouracil, 1-(2′-deoxy-2′-fluoro-β-D-arabinofuranosyl)-5-fluorouracil, doxifluridine, 5′-deoxy-5-fluorouridine, 1-acetyl-3-O-toluyl-5-fluorouracil, 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin; N-substituted deacetylvinblastine amide (vindesine) sulfates; and Cu(II)-VP-16 (etoposide) complex, pyrrolecarboxamidino-bearing etoposide analogues, 40-amino etoposide analogues, γ-lactone ring-modified arylamino etoposide analogues, N-glucosyl etoposide analogue, etoposide A-ring analogues, 4′-deshydroxy-4′-methyl etoposide, pendulum ring etoposide analogues and E-ring desoxy etoposide analogues.

Anti-cancer chemotherapeutic agents can be utilized, either with or without a “carrier,” which means a molecule which is capable of forming a covalent and/or non-covalent linkage with the chemotherapeutic agent, thereby facilitating delivery of the agent to a cell and/or tissue. Exemplary carriers include, but are not limited to, dextrans (U.S. Pat. No. 6,409,990), liposomes, polyethylene glycol based conjugates; acrylic acid based conjugates, polymers, ointments, and/or nucleic acid vectors.

G. Methods For Reducing Cell death

The invention provides methods for reducing cell death (such as reducing apoptosis), comprising: a) providing: i) cells; and ii) an agent that reduces nuclease activity of any one or more of MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II; and b) contacting the cells with the agent to produce contacted cells, wherein cell death (such as by apoptosis) of the contacted cells is reduced.

In one embodiment, the agent that reduces the nuclease activity comprises an antibody that specifically binds to any one or more of MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II; and b) contacting the cells with the agent to produce contacted cells, wherein cell death (such as by apoptosis) of the contacted cells is reduced. In another embodiment, the method further optionally comprises detecting reduced cell death (such as by apoptosis) in the contacted cells.

The invention's methods are useful in, for example, identifying compounds (e.g. environmental, chemical, natural occurring, man-made, etc.) that may alter (i.e. increase or reduce) cell death (such as by apoptosis) that is mediated by any one or more of ADH portion, MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II. Such compounds may be useful as therapeutics that reduce symptoms associated with cell death (such as by apoptosis) that is mediated by these proteins. In another embodiment, the invention methods may also be used to reduce symptoms of disease associated with reduced cell proliferation (e.g. to increase regeneration of tissue in burn victims and to reduce scarring).

In one embodiment, the contacting comprises mixing the cells with the agent, such as where the mixing is carried out in vitro. In another embodiment, the contacting comprises expressing a nucleotide sequence that encodes any one or more of MDH portion, MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II, in the cells.

The cells in the invention methods may be in vitro or in vivo in a mammalian animal, such as a human.

H. Methods For Detecting Apoptosis

The invention provides methods for detecting apoptosis, comprising detecting MADF and/or ADF in the cytoplasm of the cell, and/or in the blood (including plasma, platelets, etc.) of a mammalian animal. These methods are useful for detecting and diagnosing diseases that are associated with altered (including increased and reduced) cell apoptosis.

In a preferred embodiment, the method further comprises quantifying the level of the detected MADF and/or ADF. This may be useful in further quantifying the level of apoptosis. Several methods for detecting MADF and/or ADF are known in the art, such as detecting the binding of antibody that is specific for MADF and/or ADF to proteins in situ in the cell cytoplasm (e.g., using immunofluorescence microscopy) or in vitro in a cell extract. Where blood (including plasma, platelets, etc.) is used for detection, the blood may be in vivo or ex vivo after removing a blood sample.

I. Methods for Identifying Agents that Alter Cell death

The invention also provides methods for identifying a test agent as altering (including increasing and reducing) cell death (such as apoptosis), comprising: a) providing: i) an amino acid sequence comprising one or more of MDH portion, MADF, ADF, Htra/Omi, apoptosis inducing factor, Smac/DIABLO, EndoG, Cytochrome C, Nix, Nip3, CIDE-B, gelsolin, Bax, Bad, Bid, caspase-activated DNase, DNase I, and DNase II; and ii) test agent; b) contacting the amino acid sequence with the test agent; and c) detecting altered (including increased and reduced) DNA nuclease activity of the amino acid sequence in the presence of the test agent compared to in the absence of the test agent, thereby identifying the test agent as altering (including increasing and reducing) cell death (such as by apoptosis).

The terms “test compound,” “compound,” “agent,” “test agent,” “molecule,” and “test molecule,” as used herein, refer to any chemical entity, pharmaceutical, drug, and the like. Agents comprise both known and potential therapeutic compounds. An agent can be determined to be therapeutic by screening using the screening methods of the present invention. Agents are exemplified by, but not limited to, antibodies, nucleic acid sequences such as ribozyme sequences, organic molecules, inorganic molecules, and libraries of any type of molecule, which can be screened using a method of the invention. Methods for making these agents are known in the art, such as methods for preparing oligonucleotide libraries (Gold et al., U.S. Pat. No. 5,270,163, incorporated by reference); peptide libraries (Koivunen et al. J. Cell Biol., 124: 373-380 (1994)); peptidomimetic libraries (Blondelle et al., Trends Anal. Chem. 14:83-92 (1995)) oligosaccharide libraries (York et al., Carb. Res. 285:99-128 (1996); Liang et al., Science 274:1520-1522 (1996); and Ding et al., Adv. Expt. Med. Biol. 376:261-269 (1995)); lipoprotein libraries (de Kruif et al., FEBS Lett., 399:232-236 (1996)); glycoprotein or glycolipid libraries (Karaoglu et al., J. Cell Biol. 130:567-577 (1995)); or chemical libraries containing, for example, drugs or other pharmaceutical agents (Gordon et al., J. Med. Chem. 37:1385-1401 (1994); Ecker and Crook, Bio/Technology 13:351-360 (1995), U.S. Pat. No. 5,760,029, incorporated by reference). Libraries of diverse molecules also can be obtained from commercial sources.

J. Methods for Identifying Molecules that Increase Cell Death

The invention provides methods for identifying factors in biological samples that cause (e.g., increase) cell death (such as apoptosis), preferably in apoptosis-resistant and/or necrosis-resistant eukaryotic cells. In one aspect the method includes the incubation of cells, cell extracts or isolated nuclei of apoptosis-resistant cells biological samples to assay for the presence of cell death-inducing factors. In one embodiment, the number of different factors being exposed to the cells, cell extracts or isolated nuclei is greater than 100. If this biological sample is observed to possess cell death-inducing activity, analysis of the sample that possesses cell death-inducing activity will be done to determine the identity of the component with the activity.

In a preferred embodiment the apoptosis-resistant or necrosis-resistant eukaryotic cells are apoptosis-resistant due to an overexpression of Bcl-2. Another aspect of the invention involves using biological samples that comprise complex mixtures of biological components, and the analysis of the samples that possesses cell death-inducing activity to determine the identity of the component with the activity comprises the steps of fractionation of the extract, and testing or the fractionated extracts for cell death-inducing activity and for fractions containing cell death-inducing activity, determination of the component(s) responsible for inducing cell death. Another aspect of the invention provides a method wherein the biological samples comprise cell extracts or media from cells in which proteins have been expressed from a DNA construct that was introduced to the cells, and where the analysis of the sample that possesses cell death-inducing activity to determine the identity of the component with the activity comprises the determination of the DNA sequence of the DNA constructs associated with the sample that possesses cell death-inducing activity. This may include creating a library of the DNA constructs and introducing the library of the DNA constructs into the cells followed by screening extracts or media from the cells into which the DNA constructs have been introduced. The screening might comprise an assay for cell death-induction followed by determination of the sequence of the DNA construct that was present in the cells that correspond to the cell extracts or media that induce cell death. The extract or media from the cells into which the DNA constructs have been introduced can be derived from cells carrying a single DNA construct or multiple DNA constructs. In case of multiple DNA constructs further screening of a group of cell death-inducing DNA constructs is then performed on cells carrying single DNA constructs from the group of cell death-inducing DNA constructs, to identify which DNA constructs encodes proteins responsible for the induction of cell death. The mechanism of cell death is either apoptosis, necrosis, aponecrosis or autophagic degeneration. The cell extracts used for screening can be either from untreated cells or from UV radiation or other apoptosis, necrosis, aponecrosis-inducing agent-treated cells. The cell death-inducing molecules can be either peptides, polypeptides, proteins, lipids, oligosaccharides or small molecules.

In a preferred embodiment the eukaryotic cell extracts are human cell extracts. The cell extracts could also be extracts enriched in components from cellular organelles like mitochondria. The methods used to determine the presence of cell death-inducing factors can be a DNA fragmentation assay.

Another aspect of the invention provides a method for identifying gene products that can cause cell death in apoptosis-resistant cells, comprising the steps of introducing DNA into an apoptosis-resistant host cell where the DNA comprises all cis-acting sequences necessary to express a gene under the control of an induction system and inducing the expression of that gene followed by monitoring the host cell for indications of death and determination of the identities of the apoptosis-inducing gene products by determining the identity of the DNA constructs that cause death in the apoptosis-resistant host cells.

K. Methods for Identifying Molecules that Reduce Cell Death

The invention provides methods for identifying compounds that reduce cell death (such as apoptosis) in cells, comprising the steps of adding a molecule comprising one or more of MADF and ADF to cells or cellular extracts and assaying these extracts for markers of cell death (such as by apoptosis) and in addition to adding the molecule to the cells or cellular extracts, also adding a compound and assaying for the inhibition of cell death (such as by apoptosis) by that compound. The compounds that promote cell death (such as by apoptosis) can be identified by identifying an interaction molecule that binds to ADF in cells followed by identifying compounds that interact with the interaction molecule and assaying these compounds that can interact with the interaction molecule for their ability to promote cell death (such as by apoptosis). The identification of molecules that bind to ADF is accomplished using the two hybrid system, phage display, or other combinatorial biology methods, or using a pull-down assay followed by mass-spectrometry of pulled-down molecules, or by an in-gel or on-filter binding of ADF to electrophoretically separated cell extracts.

In one embodiment, of the methods for identifying compounds that reduce cell death (such as by apoptosis), the method comprises the steps of identifying an interaction molecule that binds to ADF in cells and identifying compounds that interact with the interaction molecule and assaying these compounds that can interact with the interaction molecule for their ability to inhibit ADF-induced cell death (such as by apoptosis). The identification of molecules that bind to ADF is accomplished using the two hybrid system, phage display, or other combinatorial biology methods, or using a pull-down assay followed by mass-spectrometry of pulled-down molecules, or by an in-gel or on-filter binding of ADF to electrophoretically separated cell extracts.

In a further embodiment, the invention provides a conjugate of a cell death-inducing molecule operably linked to a cell marker-recognizing compound wherein the cell death-inducing molecule comprises a peptide that is at least 40% or 60% or 80% identical in sequence to any peptide identified according to any of the invention's methods, over an amino acid stretch of at least 15, 12 or 10, respectively, consecutive residues in the sequence of the peptide identified according to above method or with or without insertions or deletions in the corresponding sequence of the cell death-inducing molecule.

In another embodiment of the invention, the invention provides a conjugate of a cell death-inducing molecule (such as peptide) and a cell marker-recognizing compound wherein the DNA encoding the cell death-inducing molecule can be hybridized to any DNA encoding any polypeptide identified according to the methods described herein, at 60° C. at one or more of 1.5M, 1.0M, 0.75M and 0.5 M salt.

### EXPERIMENTAL

The following examples serve to illustrate certain preferred embodiments and aspects of the present invention and are not to be construed as limiting the scope thereof.

### Example 1

### Materials and Methods

The following is a brief description of the exemplary materials and methods used in the subsequent Examples, and that may be used in other embodiments of the invention.

A. Cell Lines

U937 monocytic leukemia and the MCF-7 breast cancer lines were obtained from the ATCC. The human myeloid leukemia, HL-60 neo (transfected with empty vector) and HL-60 Bcl-2 (transfected with and overexpressing Bcl-2) were generously provided by Dr. S. Knox (Stanford University, Palo Alto, Calif.), and have been described previously (Gilbert and Knox, 1997; Wright et al., 1998). All cell lines were cultured in RPMI 1640 plus 10% FCS in the absence of antibiotics and were free of mycoplasma.

B. DNA Fragmentation Assays

DNA fragmentation in whole cells or normal nuclei isolated from U937 or MCF-7 cells was assayed by release of (3H)thymidine-labeled DNA fragments as described in detail previously (Wright et al. (1994) J. Exp. Med. 180, 2113-2123). DNA fragmentation induced by rADF treatment of isolated nuclei for four h was analyzed by agarose electrophoresis as described in detail previously (Wright et al. (1994) supra). In four h assays using isolated nuclei, the buffer was supplemented with an ATP regenerating system (2 mM ATP, 10 mM phosphocreatine, and 50 μg/ml creatine kinase).

C. Purification and Amino Acid Sequencing of ADF Derived From Commercial MDH

ADF was purified from 2.9 mg of pig heart mitochondrial MDH obtained from Worthington Enzymes, New Jersey. Protein was fractionated by gel filtration on a Tosohaas G2000SWXL 7.8 mm×300 mm HPLC column, and each fraction tested for DNA fragmentation in isolated U937 nuclei (ADF activity) and for MDH enzyme activity. Active fractions were pooled and applied to a reverse phase C4 Brownlee 2.1 mm×220 mm column equilibrated in H2O+0.0075% trifluoroacetic acid pH 6.5, and eluted with a linear gradient to 40% acetonitrile.

The active fraction was separated by electrophoresis in a 10-20% Tris-tricine SDS PAGE (Novex). The protein was electrophoretically transferred to Sequi-Blot PVDF membrane (Bio-Rad), stained with 0.2% Coomassie Blue, and the N-terminal sequences of the excised peptides were determined by Edman degradation in an ABI Procise 494 sequencer.

D. MDH Enzyme Assay

This assay measures MDH-mediated oxidation of NADH as described previously (Williamson, and Corkey, 1996). Results are reported as the decrease in absorbance at 340 nm/min.

E. Cloning, Expression, and Purification of Human rADF

Total RNA was prepared from 10×10⁶ U937 cells. The RNA (5 ug) was primed with oligo dT and reverse-transcribed with reverse transcriptase (Life Technologies, Grand Island, N.Y.) according to the supplier's protocol. The resulting cDNA was then amplified by polymerase chain reaction (PCR) using gene-specific primers. Primers used to amplify the human MDH sequence coding for the 18 kDa peptide (MDH amino acids 66-238) were (SEQ ID NO:51) 5′GAATTCGCAGATCTGAGCCACATCGAGACC-3′ (complementary to MDH codons 66-73) and (SEQ ID NO:52) 5′-GTCGACTCAGACCACCTCCGTGCCGGCCTCCTGGATC-3′ (complementary to MDH codons 229-238). Primers used to amplify the sequence coding for the 9 kDa ADF (MDH amino acids 239-339) were (SEQ ID NO:53) 5′-GAATTCAAGGCTAAAGCCGGAGCAGGCTCTGC-3′ (complementary to MDH codons 239-247) and (SEQ ID NO:54) 5′-GTCGACTCACTTCAGGGTCTTCACGAAATCTTCCCC-3′ (complementary to MDH codons 329-339). The PCR products were cloned into pBAD TOPO TA vector (Invitrogen, Carlsbad, Calif.) according to the supplier's instructions. The inserts were then excised from the vector using EcoRI and Sal I enzymes and cloned downstream of the glutathione-s-transferase (GST) fusion partner in expression vector pGEX (Amersham Biosciences, Piscataway, N.J.). Fidelity of PCR amplification and in-frame cloning were confirmed by DNA sequencing. The resulting pGEX-ADF constructs were transformed into E. coli BL21 strain (Stratagene, La Jolla, Calif.) for protein expression.

rADF and empty vector control were purified in an identical fashion. Bacteria harboring the rADF constructs or vector control were cultured under standard conditions to induce expression of GST-fusion proteins as described previously (Ausubel et al., 1995).

rADF and vector control were purified with glutathione-Sepharose beads (Pharmacia Biotech) according to the manufacturer's directions. The eluted protein was digested with thrombin to liberate rADF from the GST-ADF fusion protein. Typically, this scheme resulted in a purification factor of 16, 750 fold when comparing total starting protein from the E. coli lysate to the purified 9 kDa rADF peptide.

F. Treatment of Nuclei with rADF and Extraction of DNase Activity

U937 cells were pelleted and ice-cold lysing buffer (50 mM Tris, pH 7, NP-40 0.01%) was added to achieve a concentration of 100×10⁶ cells in 1.0 ml. Nuclei were pelleted by centrifugation at 350×g for 10 min and resuspended in 0.1 ml nuclear assay buffer (50 mM Tris, 250 mM sucrose, 10 mM MgCl, pH 7). rADF was added and the samples incubated 20 h at 37° C. As controls, rADF or normal nuclei were incubated alone. The mixture was centrifuged at 14,000×g for 5 minutes and the supernatant removed. 0.1 ml of ice-cold nuclear extraction buffer (50 mM Tris pH 8.3, 20 mM EDTA) was added to the pellet and incubated 30-60 min on ice. The mixture was centrifuged at 14,000×g for 15 min, and the supernatant removed to test in the DNase assay. DNase was assayed as described in detail previously (Wright et al., 1994) using (³H)thymidine-labeled DNA isolated from U937 cells as a substrate.

G. Western Blot for ICAD

To determine if rADF causes ICAD cleavage in nuclei, isolated U937 nuclei were treated with and without rADF exactly as described above for stimulation of DNase activity. Whole U937 cells were included as a positive control, since they are known to undergo ICAD cleavage during apoptosis. U937 cells (10×10⁶/treatment) were treated with and without UV light (0.08 J/cm²) or TNF 5 ng/ml plus cycloheximide 0.5 μg/ml and incubated 4 h, at which point over 50% of the treated cells exhibited an apoptotic morphology, yet were still >95% viable by trypan blue exclusion. Apoptotic morphology was assessed microscopically by counting the percentage of cells exhibiting two or membrane blebs as described previously (Wright et al., 1992) After cell lysis, equal amounts of protein were loaded into each lane of a 10-20% SDS polyacrylamide gradient gel. After transfer to the membrane, the blot was probed with rabbit polyclonal anti-DFF45 1:100 (Affinity BioReagents, Inc, Bolder, Colo.). The blots were then incubated with horseradish peroxidase-conjugated anti-rabbit IgG 1:20,000 (Pierce), and developed with SuperSignal West Pico Substrate (Pierce) according to the manufacturer's instructions.

H. Western Blot for Translocation of ADF

ADF was detected in normal or apoptotic HL-60 cells, 200×10⁶ cells/sample. To induce apoptosis, cells were UV light irradiated in a Stratalinker at 0.2 J/cm², and then incubated for 4 h until at least 50% of the cells were morphologically apoptotic. The cells were lysed in ice-cold extraction buffer (50 mM Tris-HCl pH 7.5, 1 mM EDTA, 10 mM KH2PO₄, 0.2% BSA, 5 mM 2-ME, 0.25 M sucrose, 0.01% digitonin). The nuclei were immediately pelleted at 500×g for 10 min. The supernatant was centrifuged at 12,000×g for 10 min, and the pellet used as the mitochondrial fraction and the supernatant as the cytosol. The gel was loaded with 300 μg of protein per lane for each sample. Samples were separated by a 1.5 mm 8-16% SDS polyacrylamide gradient gel, immunoblotted, and probed with rabbit anti-rADF IgG (20 μg/ml). This antibody was prepared by immunization with gel-purified rADF followed by purification of the antiserum using a protein A column. The fraction containing IgG was absorbed with MDH-conjugated Sepharose to partially decrease its cross-reactivity with 36 kDa MDH. The blots were incubated with secondary antibody and developed as described for the ICAD blot.

I. Expression, Purification, and Assay of Recombinant Caspase 3

Caspase 3 expression plasmid pET-23b-caspase 3 was a gift from Dr. Guy Salvesen. PET23b-caspase 3 was transformed into E. coli strain BL21(DE3)pLyS, and the expression and purification of active caspase 3 using a nickel affinity column was performed as described previously (Stennicke and Salvessen, 1999).

Caspase 3 proteolytic activity was measured using the DEVD-p-nitroanalide synthetic substrate as described in detail previously (Wright et al., 1997).

J. Immuno-depletion of Cytosol Extracts

To induce apoptosis, U937 cells were exposed to UV light 0.08 J/cm², incubated for 3 h followed by lysis at 1×10⁹ cells/ml in 50 mM Tris pH 7.5 plus 0.05% NP-40. Lysates were centrifuged at 10,000×g for 10 min, and the supernatants used for the immuno-depletions. Aliquots of 40 μl of a 50% slurry of protein A/G agarose beads (Pierce) were coated with 20 μg of anti-rADF, anti-caspase 3 (Santa Cruz Biotechnology, Santa Cruz, Calif.), or normal IgG control by mixing at 4° C. for 20 h in PBS. Coated beads were washed twice in PBS, and 100 μl of cytosol was added and mixed for 1.5 h at 4° C. Beads were pelleted and the supernatant adsorbed a second time with beads coated with the same antibody, and then tested at a final concentration of 1:5 in a 4 hr. assay of DNA fragmentation on isolated U937 nuclei supplemented with 2 mM ATP, 10 mM phosphocreatine, and 50 μg/ml creatine kinase.

K. Cloning of rADF Fused to Antennapedia Peptide (Ant)

The rADF-Ant fusion was amplified by PCR using the pGEX-ADF as template. The 5′ primer was (SEQ ID NO:55) 5′GGCCGAATTCAAGGCTAAAGCCGGAGCAGGCTCTGC3′. The 3′ primer which introduced Ant at the C-terminus of rADF was (SEQ ID NO:56) 5′-AATTGTCGACTTATTTTTTCCATTTCATGCGGCGGTTCTGAAACCAAATTTTAA TCTGGCGCTTCAGGGTCTTCACGAAATCTTCCCC-3′. The PCR product was cloned into the pGEX-4T-1 vector (Amersham Biosciences, Piscataway, N.J.) at EcoRI and SalI sites. After the DNA sequence was confirmed, the pGEX-rADF-Ant construct was transformed into E. coli BL21 (DE3) strain (Novagen, Madison, Wis.) for protein expression and purification using the same methods as for rADF.

In experiments to test the activity of ADF-Ant, the commercially available peptide, penetratin (Qbiogene, Carlsbad, Calif.) was used, which has the identical amino acid sequence as that fused to rADF in the ADF-Ant construct. Since penetratin is modified for coupling the free thiol groups, it was first inactivated by treatment with DTT, according to the manufacturer's directions.

### Example 2

### Identification of ADF as a C-Terminal Fragment of Mitochondrial MDH

In preliminary studies, the inventors observed that supernatants of normal mitochondria derived from cultured cells or freshly isolated liver cells incubated in vitro contained activity that induced internucleosomal DNA cleavage in isolated U937 nuclei. Purification of this activity from liver mitochondria revealed a partial amino acid sequence of mitochondrial malate dehydrogenase (MDH), an enzyme not known to have nuclease activity. Further studies revealed that preparations of MDH derived from porcine heart mitochondria (Worthington Enzymes) could dose-dependently induce nuclear DNA fragmentation at concentrations ranging from 2-10 μg/ml, whereas the MDH isoform purified from heart cytosol (Sigma), which is a different protein, was inactive even at 200 μg/ml. To ascertain that the DNA fragmenting activity of MDH was not due to a contaminating nuclease, the inventors purified the commercial enzyme to determine the source of the ADF activity.

MDH was fractionated by gel filtration, and each fraction was tested for both MDH enzyme activity as well as induction of DNA fragmentation in isolated nuclei. The results show that the main peak of MDH enzyme activity (fraction 2) did not correlate maximum ADF activity which eluted slightly later in fraction 4. Fractions with maximum ADF activity were further purified on a second gel filtration column followed by C4 reverse phase chromatography. ADF activity eluted in a single peak that was devoid of MDH enzyme activity. SDS PAGE analysis revealed that this fraction lacked a band of 36 kDa, the size intact MDH, but instead contained two peptides of 18 kDa and 9 kDa. Both gel-purified peptides were analyzed by N-terminal amino acid sequencing which revealed both peptides were fragments of pig mitochondrial MDH. The N-terminal sequences started at amino acid 42 and 215 of MDH for the 18 kDa and 9 kDa peptides, respectively.

Preparations of the recombinant 9 kDa peptide showed dose-dependent activation of nuclear DNA fragmentation in nuclei isolated from either U937 monocytic leukemia cells or the MCF-7 breast cancer cells in a four h assay (FIG. 2A). The activity of rADF appeared not to require caspase 3, since MCF-7 does not express this protease. In contrast, control samples purified in an identical fashion from E. coli transfected with empty vector alone were inactive. The 9 kDa rADF (hereafter referred to as rADF) also induced internucleosomal DNA fragmentation in isolated U937 nuclei (FIG. 2B). This activity was not due to contaminating DNases, because similar concentrations of rADF did not digest naked DNA. Therefore, it is likely that ADF directly or indirectly activates endonucleases present in isolated nuclei.

To examine this possibility, extracts from U937 nuclei incubated with and without rADF were tested in the DNase assay. The results shown in demonstrate that extracts from rADF-treated nuclei digested naked DNA, whereas the extracts from vector control-treated nuclei or rADF incubated without nuclei had little or no activity.

### EXAMPLE 3

### Translocation of ADF from Mitochondria to Cytosol and Nucleus during Apoptosis is Blocked by Overexpression of Bcl-2

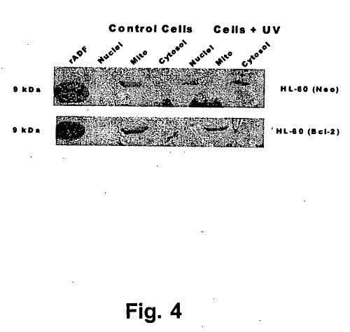

To determine whether ADF is mobilized during apoptosis, the inventors analyzed different subcellular fractions derived from normal or apoptotic HL60 cells that were transfected with empty vector control (neo) or Bcl-2. HL60 neo cells were treated with UV light and incubated for four h, at which point most of the cells exhibited an apoptotic morphology and were committed to die, yet were still >90% viable according to trypan blue exclusion. HL60 Bcl-2 cells treated in a similar fashion did not reveal significant numbers of morphologically apoptotic cells 4 h after UV light treatment, as expected. At this point, cells were lysed and fractionated by differential centrifugation to obtain nuclei, mitochondria and cytosol for immunoblotting (FIG. 4). The results revealed a 9 kDa band reacting with anti-rADF in normal mitochondria (Mito) of control cells without UV light, with no corresponding peptide visible in the cytosol or nuclear fractions. In samples prepared from apoptotic HL60 neo cells, the 9 kDa band disappeared from the mitochondria and was clearly visible in the nuclei and cytosol fractions. In contrast, all the 9 kDa peptide remained in the mitochondria of UV light-treated HL60 Bcl-2 cells. Taken altogether, these data support the hypothesis that ADF translocates from the mitochondria to the nucleus during apoptosis of sensitive cells but not Bcl-2 overexpressing apoptosis resistant cells. Thus, the anti-apoptotic properties of Bcl-2 may be due in part to the prevention of release of ADF from the mitochondria.

### Example 4

### Nuclear DNA Fragmenting Activity in Apoptotic Cytosol Extracts is Immuno-depleted by Anti-rADF

Cytosols prepared from cells committed to undergo apoptotic death have been shown to cause DNA fragmentation in isolated normal nuclei (Shimizu, et al., 1997, Leukemia 11:1238-1244; Samejima, et al., 1998, J Cell Biol 143:225-239). Based on the results of the translocation experiment, the inventors predicted that at least some of this activity might be attributed to ADF. Therefore immuno-depletion experiments were performed with antibodies prepared by immunization of rabbits with gel-purified rADF followed by purification of antiserum using protein G-Sepharose. The results in FIG. 5A show that anti-rADF removed 50% of the activity from a control sample of rADF, whereas normal IgG or anti-caspase 3 did not deplete ADF activity. Most of the nuclear DNA fragmenting activity from the apoptotic cytosol prepared from U937 cells treated with UV light was depleted by anti-rADF.

Beads coated with anti-caspase 3 did not deplete any nuclear DNA fragmenting activity, although this treatment did deplete caspase proteolytic activity measured on the DEVD-pNa synthetic substrate (FIG. 5B). The depletion of the ADF activity from activated cytosol correlated with the disappearance of the 9 kDa band reacting with anti-ADF in the immunoblot of the activated cytosol (FIG. 5B). In contrast, the 40 kDa band reacting with anti-CAD in the activated cytosol was not immuno-depleted by anti-rADF (FIG. 5C). This blot also shows that CAD is present in extracts of nuclei from normal or UV light-treated cells, but is not found in normal cytosol, as expected (Lechardeur, et al., 2000, J Cell Biol 150: 321-334). These findings support the hypothesis that ADF is a mediator of nuclear DNA fragmentation in apoptotic cytosol. The presence of ADF in apoptotic cytosols may explain previous reports indicating that caspase inhibitors could not block the nuclear DNA fragmenting activity of apoptotic cytosol (Shimizu, et al., 1997, Leukemia 11: 1238-1244), as well as the report that caspase activity could be physically separated from nuclear DNA fragmenting activity in cytosol from apoptotic cells (Samejima, et al., 1998, J Cell Biol 143:225-239).

### Example 5

### Introduction of rADF into Normal Cells Induces DNA Fragmentation Followed by Cell Death

In order to further evaluate the possible role of ADF in apoptosis, experiments were performed to determine if the introduction of rADF into normal cells would induce them to undergo DNA fragmentation. Attempts to transfect and express rADF in several cell types were unsuccessful, for reasons that are not clear. Therefore, the inventors employed an approach that has been used to deliver exogenous macromolecules fused to internalization peptides into live cells. rADF was genetically fused to penetratin and the expressed fusion (rADF-Ant) was tested for activity on U937 cells.

The results in FIG. 6A show that concentrations of rADF-Ant from 2.25-9 nM induced high levels of DNA fragmentation in a 4 h assay. In contrast, the same concentrations of unconjugated rADF combined with the free penetratin peptide (rADF+Pen) or either agent alone had little activity. DNA gel analysis revealed a typical DNA ladder induced in intact U937 cells treated with ADF-Ant, but not penetratin (FIG. 6B).

### Example 6

### Both rADF-Ant and rADF Fused to Basic Fibroblast Growth Factor (rADF-bFGF) are Cytotoxic to a Variety to Tumor Cell Types

Although the mechanism by which the antennapedia peptide internalizes into cells is not completely understood, it is thought to directly cross the cell membrane and enter the cytoplasm without interacting with membrane receptors. To determine if rADF can be delivered to cells through a receptor-ligand internalization system the inventors cloned rADF fused to 18 kD human basic fibroblast growth factor (bFGF). Receptors for bFGF are expressed on many tumor cell lines and bFGF fused to plant-derived toxins has been used to target tumor cells in experimental systems. Both fusion proteins were tested for cytotoxicity against several different human tumor cell lines in a 20 h assay measuring cell death by trypan blue exclusion. The results are shown in Table 3.

TABLE 3
Both rADF-Ant and rADF-bFGF are Potently
Toxic to Different Tumor Cell Types.

IC₅₀ Values (nM ± SEM)

| Cell | | rADF-Ant | rADF-bFGF |
|---|---|---|---|
| U937 | monocytic leukemia | 2.4 ± 0.4 | 5.3 ± 1.4 |
| MCF-7 | breast cancer | 6.7 ± 0.8 | 3.7 ± 0.2 |
| Jurkat | T cell leukemia | 1.2 ± 0.1 | 1.8 ± 0.1 |
| Raji | B cell lymphoma | 2.5 ± 1.0 | 11 ± 0.8 |

| | | |
|---|---|---|
| Mouse spleen cells | 8.3 ± 0.4 | ND |

Cells were cultured with different concentrations of the fusion proteins for 20 h and % viability determined by trypan blue exclusion, counting at least 100 cells total. Results are reported as the average of the lowest concentration to mediate 50% cytotoxicity measured in 3-4 separate assay

The results in Table 3 show that rADF-bFGF was approximately as toxic to tumor cells as rADF-Ant, with IC₅₀ values ranging from 1.8-11 nM and 1.2-6.7 nM, respectively. Of note is the fact that MCF-7 was very sensitive to the fusion proteins, even though it lacks caspase 3, which is in keeping with our previous results (submitted for publication) showing that a broad-spectrum caspase inhibitor did not prevent rADF-Ant induced cell death. The inventors also found that rADF-Ant was toxic to normal mouse spleen cells, indicating rADF will need to be specifically targeted to cancer cells to avoid damage to normal tissue. This will be accomplished by fusing ADF to single chain Hepama-1 which does not react with any normal cells or tissues tested (Fuhrer et al., 1991, Cancer Res. 51:2158-2163).

### EXAMPLE 7

### rADF-Ant is Potently Toxic to a Variety of Drug-Resistant Cancer Cell Lines

Overexpression of Bcl-2 is well-known to inhibit apoptosis and may protect cancer cells from almost all forms of therapy (Coultas et al., 2003, Semin Cancer Biol 13:115 123). Therefore, the inventors tested the susceptibility of human myeloid leukemia HL-60 cells transfected with Bcl-2 or empty vector neo control for sensitivity to ADF-Ant and etoposide-induced DNA fragmentation. Dose response assays showed that HL-60 Bcl-2 cells were highly resistant to etoposide (IC₅₀ at 380 μM) compared to HL-60 neo with an IC₅₀ of 26 μM (FIG. 7). In contrast, HL-60 Bcl-2 cells were just as sensitive to ADF-Ant as compared to HL-60 neo (IC₅₀ values of 1.8 nM and 1.4 nM, respectively). In view of our previous findings that ADF was not released from HL-60 Bcl-2 cells exposed to an apoptotic dose of to UV light (FIG. 4), these data suggest that ADF mobilization functions downstream of Bcl-2 in the normal apoptotic pathway.

Another mechanism whereby cancer cells may resist apoptosis is through overexpression of heat shock (HS) proteins which have been found at high levels in most tumor cell biopsies (Jaattela, et al., 1999, Exp Cell Res 248:30-43). The inventors found that HS treatment of U937 cells induced resistance to DNA fragmentation induced by tumor necrosis factor a (TNF), and UV light, but they were still equally sensitive to rADF-Ant (FIG. 8). We have studied the mechanisms of apoptosis resistance using several variants derived from the U937 cell line. Variants generated by prolonged growth in the presence of TNF (U9-TR) or by nutritional depletion of intracellular NAD content (U9-NAD-) have been described in the literature previously (Wright, et al., 1992, Cancer Immunol Immunother 34:399-406; Wright, et al., 1996b, J. Exp. Med. 183: 463-477). Both of these variants were resistant to apoptosis induced by diverse agents, such as chemotherapeutic drugs, UV light, and TNF. Data are shown in Table 4.

TABLE 4
U937 Variants Selected for Resistance to Apoptosis
Are Still Sensitive to rADF-Ant.

| Cytotoxic | Cell Line - IC₅₀ Value | | | |
|---|---|---|---|---|
| Agent | | U937 | U9-TR | U9-NAD - |

| | | | | | | |
|---|---|---|---|---|---|---|
| Etoposide | 12 | μM | >500 | μM | >500 | μM |
| TNF | 6 | ng/ml | >50 | ng/ml | >50 | ng/ml |
| rADF-Ant | 5 | nM | 5 | nM | 7 | nM |

DNA fragmentation elicited by the various cytotoxic agents was measured in a 4 hr assay by release of ³H-labeled DNA fragments. Results are reported as the minimal concentration of inducing agent to cause 50% DNA fragmentations

Data in Table 4 demonstrate that both U9-TR and U9-NAD were highly resistant to etoposide and TNF, with IC₅₀ values being >41 fold and >7 fold increased for etoposide and TNF, respectively. In contrast, both variants were just as sensitive to rADF-Ant as parental U937 cells. It has been documented that U937 cells differentiated to a more mature monocytic phenotype by culturing with phorbol myristate acetate (PMA) for 3-5 days become highly resistant to apoptosis (Sorbet, et al., 1999, Cell Death Differ 6:351-361), which may be dependent on activation of NF-kB (Pennington, et al., 2001, Mol Cell Biol 21:1903-1941).

TABLE 5
Drug-Resistant U937 Variants Generated by PMA-induced
Differentiation or Adherence to Fibronectin
(FN) are Still Sensitive to rADF.

| Cytotoxic | Cell Line - IC₅₀ Values¹ | | | | | | | |
|---|---|---|---|---|---|---|---|---|
| Agent | U937 | | U937 + PMA² | | U937 on BSA³ | | U937 on FN³ | |
| Etoposide | 4 | μM | 510 | μM | 4 | μM | 15 | μM |
| TNF | 1 | ng/ml | >50 | ng/ml | 3 | ng/ml | 15 | ng/ml |
| rADF-Ant | 5 | nM | 6 | nM | 9 | nM | 8 | nM |

¹Viability of normal U937 cells or PMA-treated U937 cells was determined by trypan blue exclusion after a 4 h treatment with various concentrations of the cytotoxic agents. Apoptosis of U937 cells plated on a BSA-coated plate (control) or A FN-coated plate was measured in a 4 h DNA fragmentation assay by release of ³H-labeled DNA fragments as described in detail previously.
²U937 cells were cultured for 3 days with phorbol myristate acetate (PMA) 10 nM.
³U937 cells were added to plates coated with BSA (non-specific protein control) or FN to induce adherence for 3 h, then various concentrations of the cytotoxic agents added and DNA fragmentation measured 4 h later.

The results shown in Table 5 confirm that PMA-treated cells are resistant to etoposide and TNF-induced apoptosis by a factor of 127-fold and >50-fold, respectively. However, PMA-treated cells are just as sensitive to rADF-Ant as normal U937 cells.

Adhesion of cells to the extracellular cell matrix (ECM) through integrin receptors can also induce resistance to apoptosis. Integrins are cell surface adhesion receptors composed of a and b subunits, which mediate ECM and cell-cell interactions. b1-integrin transduces signals from the extracellular environment involved in regulating growth, differentiation, invasive, and metastatic aspects of malignant cells. Disruption of the interaction between cells and the ECM can cause apoptosis and binding of integrins to the ECM can deliver anti-apoptotic signals (Ruoslahti, et al., 1994, Cell 77:477-478). Adhesion of U937 cells (Hazlehurst et al., 2001, Blood 96:1897-1903) to immobilized ECM protein such as fibronectin (FN) has been shown to induce resistance to chemotherapeutic agents. Therefore, the inventors tested U937 cells seeded on plates coated with BSA as a negative control or with FN according to a previously published method. The results shown in Table 5 demonstrate that cells adhering to FN required about 5-fold higher concentrations of etoposide or TNF to induce 50% DNA fragmentation as compared to cells plated on BSA. However, cells interacting with FN were still just as sensitive to rADF-Ant as control cells.

One of the most frequently reported alterations associated with multidrug resistance is increased expression of the 170 kDa membrane protein called P-glycoprotein encoded by the MDR1 gene. It functions as an energy-dependent drug efflux pump that reduces intracellular drug accumulation, thereby causing resistance to many structurally unrelated drugs (Roninson, et al., 1986, Proc Natl Acad Sci 83:4538-4542). To determine if MDR1 overexpressing cells were sensitive to rADF, the inventors tested the parental drug-sensitive uterine sarcoma, MES-SA, compared to a variant selected by culturing in doxorubicin, MES-SA/Dx5, described previously (Chen et al., 1997). This variant expressed high levels of P-glycoprotein and was cross-resistant to a variety of chemotherapeutic drugs. Although MES-SA/Dx5 was highly resistant to etoposide, it was just as sensitive to rADF-Ant as the parental line (Table 6).

TABLE 6
MDR1-Overexpressing Drug Resistant Variants
are Still Sensitive to rADF-Ant.

| Cytotoxic | IC₅₀ Values | | |
|---|---|---|---|
| Agent | | MES-SA (parental) | MES-SA/Dx5 (MDR1 high) |

| | | | | |
|---|---|---|---|---|
| Etoposide | 59 | μM | >500 | μM |
| rADF-Ant | 80 | nM | 78 | nM |

% Cytotoxicity mediated by different concentrations of the cytotoxic agents was measured in a 48 h MTT dye reduction assay as described previously (Wright, et al., 1992, Cancer Immunol Immunother 34: 399-406). Results are reported as the lowest drug concentration to elicit 50% cytotoxicity or else the highest concentration tested (>500 μM) that caused less than 50% cytotoxicity.

### Example 8

### rADF-Ant is More Potently Toxic to HCC Cell Lines than Standard Chemotherapeutic Drugs

The cytotoxic activity of rADF-Ant was compared to several chemotherapeutic drugs on a panel of three HCC cell lines. Each cell was tested on 3-5 occasions to determine the IC₅₀ values in a 48 h MTT dye-reduction assay. The results are shown in Table 7.

TABLE 7
Sensitivity of HCC Cell Lines to
rADF and Chemotherapeutic Drugs.

| | IC₅₀ Values ± SEM (n) | | |
|---|---|---|---|
| Drug | HepG2 | Hep3B | SK Hep-1 |
| rADF-Ant | 90 nM ± 3.5 (4) | 75 nM ± 8.5 (4) | 48 nM ± 6.2 (4) |
| 5-FU | 620 μM ± 66 (4) | 410 μM ± 57 (3) | 203 μM ± 6.0 (3) |
| Etop | 231 μM ± 62 (3) | 95 μM ± 2.5 (4) | 58 μM ± 2.5 (4) |
| Dox | 122 μM ± 10 (5) | 80 μM ± 4.5 (4) | 73 μM ± 3.7 (4) |

Cytotoxicity was measured in a 48 h MTT dye reduction assay as described previously (Wright, et al., 1992, Cancer Immunol Immunother 34: 399-406).

The results in Table 7 above show that rADF-Ant was approximately 1000-10,000-fold more toxic than 5-fluorouracil (5-FU), etoposide (Etop), or doxorubicin (Dox).

It is known that most HCC's are highly resistant to chemotherapeutic drugs, most likely due to multiple mechanisms. It has been shown that HCC's have elevated levels of MDR1 gene expression (Goldstein et al., 1988, J Natl Cancer Inst 81:116-124), and that the response to chemotherapy may be inversely related to the level of p-glycoprotein expression (Ng, et al., 2000, Am J Clin Pathol 113:355-363; Chou et al., 1997, J Gastroenterol Hepatol 12:569-575). Hep3B (Park, et al., 1994, J Natl Cancer Inst 86:700-705) and HepG2 (Takeuchi, et al., 1999, J Gastroenterol 34:351-358) express very high levels of the MDR1 gene, whereas SK Hep-1 expresses somewhat lower levels of MDR1 (Takeuchi, et al., 1999, J Gastroenterol 34:351-358).

HCC lines may also exhibit multidrug resistance unrelated to MDR1 gene expression (Shen, et al., 1991, J Cell Sci 98:317-322). Without limiting the invention to any particular mechanism, another possible mechanism of resistance is expression of the multidrug resistance associated protein (MRP). This protein is thought to exert a function similar to the p-glycoprotein, and is expressed at high levels in the HepG2 and SK Hep-1 cell lines (Takeuchi, et al., 1999, J Gastroenterol 34:351-358). Increased expression of thioredoxin, which functions as a cellular defense mechanism against oxidative stress, may also protect HCC's from some chemotherapeutic agents (Kawahara, et al., 1996, Cancer Res 56:5330-5333). Expression of different xenobiotic enzymes (e.g. cytochrome P-450 and glutathione S-transferase) may also contribute to the intrinsic drug resistance of HCC (Murray, et al., 1993, Cancer 71:36-43). Additional studies explored whether the high intrinsic levels of drug resistance in the HCC lines can be even further augmented by adherence to ECM protein. Overexpression of b1 integrin and its role in the progression of HCC has been reported (Patriarca, et al., 1993, J Pathol 171:5-11). Overexpression of b1-integrin in several HCC lines (including HepG2) protected them from chemotherapeutic drug-induced apoptosis via a MAP kinase-dependent pathway (Zhang, et al., 2002, Cancer 95:896-906). Knockout of a6b1-integrin expression reversed the transformed phenotype of HepG2 cells (Carloni et al., 1998, Gastroenterology. 115:433-442). The fact that almost all surgically resected non-fibrolamellar HCCs showed overexpression or abnormal expression of fibronectin (FN) (Torbenson, et al., 2002, Mod Pathol 15:826-830) suggests that this may lead to abnormal cell-cell or cell-ECM interaction, thus contributing to tumor development. Furthermore, HCC lines such as HepG2 and Hep3B were shown to secrete mediators that stimulate collagen synthesis in myofibroblasts (Faouzi et al., 1999, J Hepatol 30:275-284). These properties of HCCs may contribute to the marked changes in amount and distribution of ECM components found in the stroma of HCC as compared to normal liver (Jaskiewicz, et al., 1993, Anticancer Res 13:2229-2238). Thus, the combination of increased expression of integrins in HCCs as well as abnormalities in the surrounding ECM may create an environment that both promotes tumor growth and inhibits apoptosis. Therefore, the inventors tested HepG2 and Hep3B for drug sensitivity when assayed on plates coated with BSA as a control, or on FN as described above for U937 cells. The results are shown in Table 8.

TABLE 8
HCC Cells Rendered Resistant to Chemotherapeutic Drugs
by Adherence to FN are Still Sensitive to rADF-Ant.

IC₅₀ Values - Cell Line (plate coating)

| HepG2 | | Hep3BDrug | | |
|---|---|---|---|---|
| (BSA) | (FN) | | (BSA) | (FN) |

| | | | | | | | | |
|---|---|---|---|---|---|---|---|---|
| 5FU | 515 | μM | >2 | μM | 490 | μM | >2 | μM |
| Dox | 104 | μM | >400 | μM | 86 | μM | >400 | μM |
| Etop | 35 | μM | 1 | μM | 210 | μM | 840 | μM |
| rADF-Ant | 86 | nM | 50 | nM | 62 | nM | 64 | nM |

Microtiter plates were coated with either BSA or FN and allowed to dry as described previously (Hazlehurst et al., 2001, Blood 96: 1897-1903) before adding cells and different concentrations of drugs. % cytotoxicity was determined in a 48 h MTT dye reduction assay as described previously (Wright, et al., 1992, Cancer Immunol Immunother 34: 399-406).

Table 8 shows that HepG2 and Hep3B cells plated on FN were highly resistant to 5-fluorouracil (5FU), doxorubicin (Dox) and etoposide (Etop) as compared to the BSA control. In contrast, interaction with FN did not alter the cells' sensitivity to rADF-Ant.

### Example 9

### Generation of HCC-Specific Antibody

Hepama-1 was produced by immunizing mice with the crude cell membranes derived from the tumorigenic human HCC cell line, BEL-7402 (Fuhrer et al., 1991, Cancer Res. 51:2158-2163). Hybridomas were prepared by standard technology, and culture supernatants of clones were screened by primary ELISA against the BEL-7402 membrane preparation. In a secondary screen, one clone (Hepama-1, IgG2b) bound BEL-7402 but not normal liver cells. Hepama-1 was subcloned and ascites prepared and purified by protein A affinity chromatography for further studies.

### Example 10

### Characterization of Antibody Binding

Indirect immunofluorescence showed that Hepama-1 bound to five out of six human HCC cell lines examined (Fuhrer et al., 1991, Cancer Res. 51:2158-2163) but not to human colon (HT29, Calu 1) or breast (MCF-7, BT-20, BT549) cancer cell lines. Immunoperoxidase staining by Hepama-1 was carried out on thin sections of paraffin-embedded human tumor biopsies and normal tissues. All eight liver carcinomas were positive, whereas no staining was observed on lymphomas or carcinomas of the lung, breast, omenium, bronchus or colon. On a panel of normal tissues, Hepama-1 bound to fetal liver (no binding to normal adult liver tissue), showed trace binding to fetal lung (no binding to normal adult lung tissue), and essentially did not bind to any other fetal or adult tissues (colon, stomach, trachea, muscle, etc.), with weak reactivity to adult kidney and gall bladder. Immunocytochemical analysis showed that Hepama-1 binds to the surface of fixed cells with no evidence of cytoplasmic binding. Most importantly, Hepama-1 has been shown to internalize and deliver the toxin, trichosanthin to tumor cells and kill them (Wang, et al., 1991, Cancer Res 51:3353-3355)

### Example 11

### Antigen Characterization

The antigen is located on the cell surface and has an apparent molecular weight of 43 kD, as visualized by Western blotting with Hepama-1. Because the antibody reacted with all HCC biopsy specimens, fetal liver and lung, but not with normal adult tissues (e.g. liver, breast, colon or stomach), the antigen appears to be an onco-fetal protein. The molecular identity of the antigen is as yet unknown.

### Example 12

### Xenograft Data

Hepama-1 was conjugated to ¹³¹I and tested in a mouse HCC xenograft model by Song (Song, et al., 1998, Cell Res. 8:241-247), who showed specific localization of the antibody to the tumor, and also reported that the treatment caused tumor cytoreduction and a significant increase in survival time.

### Example 13

### Summary of Human Studies

Hepama-1 has been evaluated in more than 100 patients at the Liver Cancer Institute of the Zhongshan Hospital at the Shanghai Medical University. Initial clinical trials in humans using murine ¹³¹I-Hepama-1 have shown specific tumor localization, cytoreduction and improved survival.

Radioimmunotherapy (RIT) with ¹³¹I-labelled Hepama-1 has been conducted over the last 13 years at numerous hospitals in China including ones in Shanghai, Suzhou, Hangzhou, Henan, Guangzhou and Beijing. Early clinical trials utilized delivery through the hepatic artery, in combination with ligation, and resulted in significant tumor cytoreduction.

One published study (Zeng, et al., 1998, J Cancer Res Clin Oncol 124:275-280) at the Zhongshan hospital examined 65 patients with non-resectable HCC, treated with either ¹³¹I-labelled Hepama-1 or chemotherapy (5-fluorouracil, cisplatin or doxorubicin) delivered by the same method. The ¹³¹I-Hepama-1 was well-tolerated, with no indications of adverse reactions even at the highest dosing (100 mCi/patient), in contrast to chemotherapy. In most patients treated with ¹³¹I-Hepama-1, cytoreduction of tumors was observed. For the RIT group, this led to 53% of the patients becoming resectable, as opposed to only 9.1% in the chemotherapy-treated patients. The higher resection rate in the RIT group presumably contributed to increased survival at 5 years, as shown in Table 9 below:

TABLE 9
Treatment and survival of patients by intrahepatic artery-delivered
¹³¹I-Hepama-1 (RIT) or chemotherapy (non-RIT).

| | | |
|---|---|---|
| Treatment and Survival | Non-RIT | RIT |

| | | | | |
|---|---|---|---|---|
| Resected after treatment* | 9.1% | (3/33) | 53.1% | (17/32) |
| Survival - 1 year | 45% | (15/33) | 50% | (16/32) |
| 3 year | 12% | (4/33) | 34% | (11/32) |
| 5 year** | 9% | (3/33) | 28% | (9/32) |

*P < 0.01;
**P < 0.05

Five year survival rates for non-resectable HCC without treatment are 5-10% according to historical controls (Venook, et al., Curr Treat Options Oncol. 2000 December;1(5):407-15).

A recently completed study examined the effect of intravenous delivery of various doses of ¹³¹I-Hepama-1 on 32 non-resectable HCC patients. A detailed report of this phase I/IIa trial has been submitted to the State Drug Administration in the People's Republic of China. The report shows no indication of adverse reactions in patients treated with even the highest dose of 100 mCi (specific activity 6-7 mCi/mg antibody). Whole body dosimetry shows specific tumor-binding of the antibody in 78% (25/32)of the patients.

### Example 14

### Synthesis of the Exemplary scFv Construct and scFv Fused to ADF

The goal of the following examples is to determine if a single chain Hepama-1 genetically fused to the toxic ADF peptide will selectively kill HCC lines. The scFv will be prepared by gene assembly based on the sequence of the Hepama-1 hybridoma. The binding specificity and internalization of the scFv as compared to Hepama-1 as positive control will be evaluated by cell-based ELISA. The scFv-ADF construct (TH101) will be prepared and also tested for HCC binding and internalization. It will then be tested for toxicity against HCC lines as well as unrelated tumor types and normal cells. Radiolabeled TH101 will be evaluated for HCC-selective accumulation as compared to normal liver in a murine HCC xenograft model. The results of this study will provide the basis to proceed to evaluate TH101 in murine models of HCC in future work.

The single-chain Hepama-1 coding sequence will be produced by a standard gene assembly method (Stemmer, et al., 1995, Gene 16:49-53). The amino acid sequence of the expressed protein will be (SEQ ID NO:80) QVHLIQAGPGLVQPSQSLSITCTVSGLSLINYGVHWVRQSPGKGLEWLGVIWSGG STDYNAAFI SRLSISKDNSKSQVFFKMNSLQGNDTAIYYCARNSELGAMDYWAQGISVTVSSG GGGSGGGGSGGGGSDIVLTQSPASLAVSLGQRATISCRASKSVSTSGYSYMHWN QQKPGQPPRLLIYLVSNLESGVPARFSGSGSGTDFTLNIHPVEEEDAATYYCQHIR EAYTFGGGTKLEIK.

The first and last blocks are the variable regions of the heavy and light chains, respectively, which are separated by a (GGGS)₃ linker.

Reverse translation of this sequence, using the preferred E. coli codons, and adding appropriate restriction sites for cloning into pET vectors (see below), gives the following DNA sequence (SEQ ID NO:81), in the 5′ to 3′ direction (top strand shown, initiating ATG in italics and bold, underlined sequence is the scFv; other sequences are present for the purposes of cloning/expression):

GCAATACTCC [symbol] GGCCAGGTGCATCTGATTCAGGCGGGCCCGGGCCTGG
TGCAGCCGAGCCAGAGCCTGAGCATTACCTGCACCGTGAGCGGCCTGAGC
CTGATTAACTATGGCGTGCATTGGGTGCGTCAGAGCCCGGGCAAAGGCCT
GGAATGGCTGGGCGTGATTTGGAGCGGCGGCAGCACCGATTATAACGCGG
CGTTTATTAGCCGTCTGAGCATTAGCAAAGATAACAGCAAAAGCCAGGTG
TTTTTTAAAATGAACAGCCTGCAGGGCAACGATACCGCGATTTATTATTG
CGCGCGTAACAGCGAACTGGGCGCGATGGATTATTGGGCGCAGGGCATTA
GCGTGACCGTGAGCAGCGGCGGCGGCGGCAGCGGCGGCGGCGGCAGCGGC
GGCGGCGGCAGCGATATTGTGCTGACCCAGAGCCCGGCGAGCCTGGCGGT
GAGCCTGGGCCAGCGTGCGACCATTAGCTGCCGTGCGAGCAAAAGCGTGA
GCACCAGCGGCTATAGCTATATGCATTGGAACCAGCAGAAACCGGGCCAG
CCGCCGCGTCTGCTGATTTATCTGGTGAGCAACCTGGAAAGCGGCGTGCC
GGCGCGTTTTAGCGGCAGCGGCAGCGGCACCGATTTTACCCTGAACATTC
ATCCGGTGGAAGAAGAAGATGCGGCGACCTATTATTGCCAGCATATTCGT
GAAGCGTATACCTTTGGCGGCGGCACCAAACTGGAAATTAAACTCGAGGC
ATAGCC

Primers encoding the entire top and bottom strands for this molecule, of about 40 nucleotides each, and wherein the oligos on the top and bottom strands are staggered so as to be able to self-prime each other, have been designed. The entire set of primers will be mixed together at a concentration of 50 nM each and extended using taq polymerase, and thermally cycled for 55 cycles. Subsequently, the resulting amplified mixture will be diluted 40-fold into a PCR reaction using 2 primers (1 uM each) that amplify the full-length construct (20 cycles). In our experience, this protocol robustly yields a single DNA band of the expected size.

The DNA fragment will then be cloned into pET22a for periplasmic expression. Generally several clones (about 10) need to be sequenced to find one that lacks any errors, due to imperfect chemical synthesis. pET22a encodes a version of the scFv that has an E. coli signal peptide, directing expression in the periplasm. The vector also encodes a C-terminal hexahistidine tag. This is the standard approach for producing correctly folded, active antibody fragments, with properly formed disulfide bonds in bacteria, and has already been used successfully for this scFv by Xie et al (personal communication).

In some cases, the yield of periplasmic scFv can be low, so the scFv-coding sequence will also be cloned using the same vector but a different 5′ restriction site, thus deleting the signal peptide, for cytoplasmic expression. These antibody fragments will probably be expressed in inclusion bodies. Subsequent to expression, the inclusion bodies will be isolated and washed, and the proteins solubilized in 6M guanidinium HCl. Under these denaturing conditions, the scFv will be purified using Ni-NTA agarose, eluted with imidazol, and then refolded using continuous dialysis to slowly reduce the denaturant concentration, and simultaneously allow for spontaneous oxidation of the disulfide bonds. The starting protein concentration will be 0.1 mg/ml, to avoid excessive precipitation during refolding, and the final dialysis buffer will be PBS plus 0.1% Tween-20 and 10% glycerol. This type of protocol has also been extensively described in the literature. The soluble fraction of protein after this dialysis procedure will be analyzed for activity by ELISA against HCC cells versus other cell lines as controls. Detection of scFv binding to the immobilized cells will be done using an anti-his-tag antibody-HRP conjugate (commercially available).

To create a construct for the expression of an scFv-ADF fusion protein, the ADF-coding sequence (described above) will be cloned into the preferred pET-HCC-scFv vector (depending on which construct works better in terms of expression and HCC binding), such that ADF will be appended to the C-terminus of the scFv, separated by a GSG linker. The rationale for this is that the C-terminus of an scFv is on the opposite side of the antibody fragment from the antigen-binding site (combining region). The hexahistidine tag may be maintained for purification/immunodetection purposes. This fusion protein will be produced and purified in the same manner as for the scFv alone. This chimeric protein will then be tested for its ability to kill HCC cell lines, as compared to non-HCC controls.

### Example 15

### Cell-Binding Studies

Initially, binding of Hepama-1, scFv, scFv-ADF and normal mouse IgG control will be measured to the cell surface of three HCC cell lines: HepG2, Hep3B, and SMMC-7721. Different concentrations of antibodies will be incubated with the cells on ice for 0.5, 1, 2, and 3 hr followed by washing to remove unbound antibody and development in the ELISA assay. This will determine the optimal conditions (antibody concentration and time) to obtain maximal binding to the cell surface. These conditions will be used to evaluate the binding specificity of our new constructs.

Binding specificity will be evaluated by cold competition and by use of different cell types. Hepama-1 and the control monoclonal anti-transferrin receptor antibody will be tested for competition at 100 fold excess concentrations compared to scFv and TH101. Since these monoclonals do not have the His tag, they will not produce a signal with the secondary anti-His-tag-HRP antibody used in the ELISA. It is predicted that Hepama-1, but not anti-transferrin receptor antibodies, will inhibit binding of scFv and TH101 to the HCC cell lines.

We will then evaluate the tumor cell specificity of scFv and TH101 binding under the optimal conditions as determined above. The binding to non-HCC human tumor cell lines will be tested on the MCF-7 breast carcinoma, OVCAR-3 ovarian carcinoma, DU145 prostate cancer, and AsPC-1 pancreatic cancer (all lines obtained from the ATCC). Also, some non-transformed human cell lines will be tested, including HUVEC (human umbilical venous endothelial cells), primary liver cell lines and normal skin fibroblast cell lines (all purchased from Clonetics, San Diego). Based on previous results with Hepama-1 (Fuhrer et al., 1991, Cancer Res. 51:2158-2163), the inventors expect that there will be little or no binding of our constructs on the non-liver tumor cells or the normal cells.

The ability of scFv and TH101 to internalize will be tested on the HepG2, Hep3B and SMMC-7721 cells using the ELISA assay. Initially, the constructs will be incubated with the cells on ice using the optimal conditions as determined above. Cells are then warmed to 37° to allow internalization for different lengths of time. Cells are then washed with a low pH buffer to remove any residual surface antibody, followed by fixation and development in the ELISA. Cytotoxicity of TH101 against the HCCs, as described below, will also be an indirect measurement of cell internalization.

An exemplary cell-based ELISA assays that may be used is as follows. The assay is set up in triplicate in a flat bottom microtiter plate. Cells are plated at 50-100,000 cells/well in 200 ml complete medium and cultured for 24 h until confluent. Cells are washed twice with warm PBS+1% FCS+0.02% NaN₃. 50 ml of different concentrations of the primary antibody ranging from 1-50 mg/ml (Hepama-1, scFv, or TH101) in PBS+2% dried skim milk are added and incubated 1 h on ice. Normal mouse IgG will be used as a negative control. After washing 3 times with PBS+0.05% Tween 20, 50 ml of horseradish peroxidase (HRP)-conjugated secondary antibody at 1 mg/ml is added and incubated 1 h on ice. Plates are washed 3 times with PBS+0.05% Tween 20, and Add 50 ml/well of TMB substrate is added and incubated at room temperature for 5-10 min, or until the background controls turn slightly yellow. The reaction is stopped by the addition of 1N H2SO₄, and the absorbance is read at 450 nm. Preliminary studies to evaluate the efficacy of this assay demonstrated good binding of a monoclonal antibody directed against the transferrin receptor to viable SKHep-1 cells.

To measure internalization, the antibodies are first bound to cells on ice at the pre-determined optimal conditions. Unbound antibody is then removed by washing twice with cold PBS. Culture media is added and cells are warmed to 37° C. to allow internalization for different lengths of time (1-4 h). Residual surface antibody is then removed with a relatively mild acid wash buffer to minimize cell lysis as described previously (Yao, et al., 2001, J Nucl Med 42:1538-1544). Culture media is removed and 100 ml of cold 0.028 M sodium acetate, 0.12 M NaCl, 0.02 M sodium barbital pH 3.0 and incubated 6 min on ice. The supernatant is removed, the cells washed with PBS, and then fixed with 0.5% glutaraldehyde in PBS for 30 min at room temperature as described previously to detect internalized immunotoxins (Di Lazzaro et al., 1994, Cancer Immunol Immunother 39:318-324). Fixative is removed, the cells washed with PBS, the secondary antibody is added, and the ELISA is developed as above.

### Example 16

### Testing Toxicity of TH101 on Cell Lines In Vitro

We will evaluate the toxic activity of TH101 as well as its specificity on the same cell lines used in the binding studies including HCC and non-HCC tumors as well as non-transformed cell lines. Cytotoxicity will be assessed using a 48 hr MTT dye reduction assay. This assay is in routine use in this laboratory and was used to generate the IC₅₀ values for the HCC cells lines in the Preliminary studies. This assay measures the ability to reduce the dye, 3-(4,5-dimethylthiazol-2-yl)-2,5-diphenyltetrazoliumbromide (MTT). It detects both necrotic and apoptotic forms of death and will be performed as described in detail previously (Wright, et al., 1992, Cancer Immunol Immunother 34:399-406). Since the MTT assay is an indirect measure of cell death, i.e. it measures cell metabolism, in select assays cell death will be verified by microscopic examination in the presence of trypan blue.

### Example 17

### Testing TH101 In Vivo for Acute Toxicity and Preferential Accumulation in Tumor in a Mouse Xenograft Model of Human HCC

The goal of these studies is to evaluate the potential toxicity of TH101 in normal mice, as well as the preferential accumulation of TH101 in tumor of HCC xenograft-bearing mice. The results will provide the basis for future studies to test TH101 in therapeutic models of HCC xenografts in mice.

A. Acute Single Dose Toxicity Study.

The goal of these preliminary studies is to estimate the maximum tolerated does (MTD) of TH101 in normal Balb/c mice. Initially a “step up step down” study will be performed to determine the approximate lethal dose.

Mice (2/group) will be given a single i.v. injection of TH101 starting at 10 mg/kg. The mice will be observed for clinical signs or mortality for 24 h. The approximate lethal dose is defined as the lowest dose that kills at least one mouse during the overnight observation period. If the initial dose is too toxic or not toxic enough, the dose will be decreased or increased two-fold and repeated until the approximate lethal dose is estimated. Based on that dose, groups of 5 mice will be given graded decreasing doses to estimate the MTD which is defined as the dose that may produce observable but not severe clinical signs, and no more that occasional mortality during the overnight observation period. On days 1, 2, and 3 after mice receive the estimated MTD, peripheral blood samples will be collected and assayed for the liver enzymes, alanine aminotransferase and aspartate aminotransferase using kits obtained from Sigma.

B. Xenograft Model to Evaluate Tumor-Specific TH101 Accumulation.

The effect of the radiolabeled hu-Hepama-1 antibody will be tested on the growth of xenografted SMMC-7721 human hepatoma, since the murine Hepama-1 antibody was shown previously to bind this cell line (Fuhrer et al., 1991, Cancer Res. 51:2158-2163), and was therapeutic in the SMMC-7721 xenograft model (Song, et al., 1998, Cell Res. 8:241-247). TH101 will be labeled with ¹²⁵I using Iodogen according to the manufacturer's (Pierce) instructions. These studies will be conducted in female athymic nude mice (6-8 weeks old) bearing established SMMC-7721 tumors. This xenograft model has been well-characterized and will be performed as described previously (Yang, et al., 2001, World J Gastroenterol. 7:216-221). Tumor cells will be implanted into the right flank of mice by subcutaneous injection of 2.5×10⁶ cells. When the tumors reach a size of 5-10 mm in diameter, the mice (groups of 5 for each time point) will be injected i.v. with ½ the MTD of radiolabeled TH101. Control non-tumor-bearing mice will also be studied. On days 1, 2, 3, and 7, following injection, mice will be killed and samples of tumor, liver, spleen, kidney, lung, bone, and, blood, will be removed and weighed. Radioactivity will be measured and the data expressed as % of injected dose (ID)/g tissue.

To optimize expression and/or refolding of the scFv-ADF fusion construct, the order of the two fragments may be reversed, such that the construct will have ADF at the N-terminus, followed by a (GGSG)₃ linker, followed by the scFv. The extended linker should allow the antibody-combining site to dock onto the antigen with minimal interference by the ADF.

While the scFv that is expressed in the bacteria may be in inclusion bodies, it is the inventors' view that the active scFv may be obtained from inclusion bodies since a similar scFv construct based on the Mab95 sequence was successfully refolded from extracts of inclusion bodies (Xie, et al., 2003, Submitted for publication).

It is the inventors' opinion that the fusion of the scFv to ADF will result in the smallest (34 kDa) immunotoxin produced to date. This property will help to overcome the transport barriers in solid tumors encountered by larger molecules (Jain, et al., 1994, Sci Am 271:58-65).

For the cell-based ELISA assays, an anti-His-HRP secondary antibody will be used to detect the His-tagged scFv and TH101 constructs. To further optimize results, biotinylated constructs and streptavidin-HRP may be used. Alternatively, the constructs will be iodinated with Iodogen, thus eliminating the secondary antibody step.

It is the inventors' opinion that the scFv will be readily internalized, because the intact antibody internalizes, and there is precedent that monovalent tumor-specific scFvs can still internalize (Gao et al., 2003, J Immunol Methods 274:185-197). Also, it was shown previously that a scFv form of an antibody directed against the Ley antigen genetically fused to Pseudomonas exotoxin A could internalize in tumor cells just as effectively an the intact antibody (Siegall, et al., 1994, J Immunol 152:2377-2384).

There are different pathways of internalization, depending on the ligand and receptor, and the inventors cannot predict how TH101 will be internalized. It is possible that it may be delivered to the lysosomes where low pH may inactivate it. However, the inventors have already determined that incubation of rADF-Ant in vitro at pH 4.8 at 37° C. for 3 hr did not cause any loss of activity.

Even if the scFv construct is found to have less affinity for HCC that the original Hepama-1, however, it is the inventors' opinion that it may still be an effective therapeutic. Alternatively, in subsquent studies different constructs may be produced with higher affinity and greater efficacy, e.g. diabodies, minibodies, etc. (reviewed in Reff, et al., 2001, Crit Rev Oncol/Hematol 40:25-35).

Further clinical development is desirable following successful cloning and expression of the TH101 construct (Example 14), demonstration of TH101-mediated cytotoxicity on HCC cell lines with minimal toxicity to non-transformed cells (Example 15), and demonstration of little or no toxicity of TH101 to livers of normal mice and preferential accumulation in tumors of mice bearing HCC xenografts (Example 16).

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiment, it should be understood that the invention as claimed should not be unduly limited to such specific embodiment. Indeed, various modifications of the described modes for carrying out the invention which are obvious to those skilled in the art and in fields related thereto are intended to be within the scope of the following claims.

1. A composition comprising an isolated amino acid sequence that comprises a portion of SEQ ID NO:4, wherein said portion comprises SEQ ID NO:6 and has activity chosen from DNA nuclease activity and cell killing activity.

2. The composition of claim 1, wherein said portion comprises SEQ ID NO:7.

3. A composition comprising a conjugate that comprises an amino acid sequence comprising SEQ ID NO:6, wherein said amino acid sequence is operably linked to a first molecule that specifically binds to a cell molecule.

4. The composition of claim 3, wherein said amino acid sequence comprises SEQ ID NO:7.

5. The composition of claim 3, wherein said amino acid sequence further comprises a N-terminal signal peptide.

6. The composition of claim 3, wherein said amino acid sequence further comprises a cell internalization peptide.

7. The composition of claim 3, wherein said amino acid sequence further comprises a nuclear localization peptide.

8. The composition of claim 3, wherein said first molecule comprises an antibody.

9. The composition of claim 8, wherein said antibody specifically binds to cancer cells.

10. The composition of claim 9, wherein said cancer cells are chosen from non-small cell lung carcinoma cells, breast cancer cells, gastrointestinal cancer cells, renal carcinoma cells, and liver cancer cells.

11. The composition of claim 10, wherein said cancer cells comprise liver cancer cells.

12. The composition of claim 11, wherein said liver cancer cells comprise hepatocellular cancer cells.

13. The composition of claim 11, wherein said antibody that binds to liver cancer cells comprises an antibody chosen from Hepama-1, anti-PLC1, anti-PLC2, K-PLC1, K-PLC2, K-PLC3,49-D6,7-E10, 34-A4,26-A10, 34-B9,79-C8,16-E10, 5D3, 5C3, 2C6, a-AFP, H hHP-1, mAb 95, YPC2/38.8, P215457, PM4E9917, HAb25, HAb27, KY-1, KY-2, KY-3, 9403 Mab, KM-2, S1, 9B2, IB1, A9-84, SF-25, AF-10, XF-8, AF-20, a-hIRS-1, FB-50, SF 31, SF 90, 2A3D2, and 2D11E2.

14. The composition of claim 11, wherein said antibody that binds to liver cancer cells comprises Hepama-1 antibody.

15. The composition of claim 14, wherein said antibody comprising Hepama-1 antibody is humanized.

16. The composition of claim 9, wherein said cancer cells are chosen from B cell lymphoma cells, myeloid leukemia cells, renal carcinoma cells, colon cancer cells, pancreatic cancer cells, colorectal cancer cells, ovarian cancer cells, and prostate cancer cells.

17. The composition of claim 3, wherein said first molecule comprises a ligand of a cell receptor.

18. The composition of claim 17, wherein said ligand comprises a growth factor.

19. The composition of claim 18, wherein said growth factor is chosen from epidermal growth factor, insulin-like growth factor, fibroblast growth factor, and vascular endothelial growth factor.

20. A composition comprising a nucleic acid sequence encoding an amino acid sequence that comprises a portion of SEQ ID NO:4, wherein said portion comprises SEQ ID NO:6, and wherein said amino acid sequence has activity chosen from DNA nuclease activity and cell killing activity.

21. The composition of claim 20, wherein said portion comprises SEQ ID NO:7.

22. The composition of claim 20, wherein said amino acid sequence further comprises one or more of N-terminal signal peptide, cell internalization peptide, nuclear localization peptide, and an antibody that specifically binds to biotin.

23. A composition comprising an expression vector that comprises a nucleic acid sequence encoding an amino acid sequence that comprises a portion of SEQ ID NO:4, wherein said portion comprises SEQ ID NO:6, and wherein said amino acid sequence has activity chosen from DNA nuclease activity and cell killing activity.

24. The composition of claim 23, wherein said portion comprises SEQ ID NO:7.

25. A cell comprising an expression vector that comprises a nucleic acid sequence encoding an amino acid sequence that comprises a portion of SEQ ID NO:4, wherein said portion comprises SEQ ID NO:6, and wherein said amino acid sequence has activity chosen from DNA nuclease activity and cell killing activity.

26. The composition of claim 25, wherein said portion comprises SEQ ID NO:7.

27. A composition comprising an antibody that specifically binds to SEQ ID NO:7.

28. The composition of claim 27, wherein the binding affinity of said antibody to SEQ ID NO:7 is higher than the binding affinity of said antibody to SEQ ID NO:4.

29. The composition of claim 27, wherein the binding affinity of said antibody to SEQ ID NO:6 is higher than the binding affinity of said antibody to SEQ ID NO:4.

30. The composition of claim 27, wherein said binding reduces SEQ ID NO:7 activity chosen from DNA nuclease activity and cell killing activity.

31. A method for increasing cell death, comprising:
a) providing:
i) cells; and
ii) a composition comprising an amino acid sequence comprising SEQ ID NO:6; and
b) contacting said cells with said composition to produce contacted cells wherein said contacting increases cell death of said contacted cells.

32. The method of claim 31, wherein said amino acid sequence comprises SEQ ID NO:7.

33. The method of claim 31, wherein said amino acid sequence further comprises an antibody that specifically binds to said cells.

34. The method of claim 31, wherein said method further comprises detecting increased cell death in said contacted cells.

35. The method of claim 31, wherein said method further comprises, prior to step b), providing a nucleotide sequence encoding said amino acid sequence, and expressing said nucleotide sequence in said cells.

36. The method of claim 31, wherein said cells are in vitro.

37. The method of claim 31, wherein said cells are in vivo in a mammalian animal.

38. The method of claim 37, wherein said mammalian animal is human.

39. The method of claim 38, wherein said human is chosen from a human that has cancer and a human that is suspected of being capble of developing cancer.

40. The method of claim 39, wherein said amino acid sequence further comprises an antibody that specifically binds to cancer cells in said cancer.

41. The method of claim 39, wherein said cancer is chosen from liver cancer, gastric cancer, head cancer, neck cancer, lung cancer, breast cancer, prostate cancer, cervical cancer, pancreatic cancer, colon cancer, ovarian cancer, stomach cancer, esophagus cancer, mouth cancer, tongue cancer, gum cancer, skin cancer, muscle cancer, heart cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, gall bladder cancer, ocular cancer, joint cancer, glioblastoma, mesothelioma, lymphoma, leukemia, melanoma, squamous cell carcinoma, osteosarcoma, and Kaposi's sarcoma.

42. The method of claim 41, wherein said cancer is liver cancer.

43. The method of claim 42, wherein said antibody that specifically binds to liver cancer cells comprises Hepama-1 antibody.

44. A method for detecting cell apoptosis, comprising detecting SEQ ID NO:7 in the cytoplasm of said cell.

45. The method of claim 44, wherein said method further comprises quantifying the level of the detected SEQ ID NO:7.

46. A method for detecting disease in a mammalian animal, comprising detecting SEQ ID NO:7 in the blood of said mammalian animal.

47. The method of claim 46, wherein said disease is associated with cell death.


SEQUENCE LISTING (81 sequences)

SEQ ID NO: 1 - protein, 314 aa, Sus scrofa
AKVAVLGASGGIGQPLSLLLKNSPLVSRLTLYDIAHTPGVAADLSHIETRATVKGYLGPEQLPDCLKGCDVVVIPAGVPRKPGMTRDDLFNTNATIVATLTAACAQHCPDAMICIISNPVNSTIPITAEVFKKHGVYNPNKIFGVTTLDIVRANAFVAELKGLDPARVSVPVIGGHAGKTIIPLISQCTPKVDFPQDQLSTLTGRIQEAGTEVVKAKAGAGSATLSMAYAGARFVFSLVDAMNGKEGVVECSFVKSQETDCPYFSTPLLLGKKGIEKNLGIGKISPFEEKMIAEAIPELKASIKKGEEFVKNMK

SEQ ID NO: 2 - protein, 72 aa, Sus scrofa
KAKAGAGSATLSMAYAGARFVFSLVDAMNGKEGVVECSFVKSQETDCPYFSTPLLLGKKGIEKNLGIGKISP

SEQ ID NO: 3 - protein, 100 aa, Sus scrofa
KAKAGAGSATLSMAYAGARFVFSLVDAMNGKEGVVECSFVKSQETDCPYFSTPLLLGKKGIEKNLGIGKISPFEEKMIAEAIPELKASIKKGEEFVKNMK

SEQ ID NO: 4 - protein, 338 aa, Homo sapiens
MLSALARPASAALRRSFSTSAQNNAKVAVLGASGGIGQPLSLLLKNSPLVSRLTLYDIAHTPGVAADLSHIETKAAVKGYLGPEQLPDCLKGCDVVVIPAGVPRKPGMTRDDLFNTNATIVATLTAACAQHCPEAMICVIANPVNSTIPITAEVFKKHGVYNPNKIFGVTTLDIVRANTFVAELKGLDPARVNVPVIGGHAGKTIIPLISQCTPKVDFPQDQLTALTGRIQEAGTEVVKAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 5 - DNA, 1017 bp, Homo sapiens
atgctctccgccctcgcccggcctgccagcgctgctctccgccgcagcttcagcacctcggcccagaacaatgctaaagtagctgtgctaggggcctctggaggcatcgggcagccactttcacttctcctgaagaacagccccttggtgagccgcctgaccctctatgatatcgcgcacacacccggagtggccgcagatctgagccacatcgagaccaaagccgctgtgaaaggctacctcggacctgaacagctgcctgactgcctgaaaggttgtgatgtggtagttattccggctggagtccccagaaagccaggcatgacccgggacgacctgttcaacaccaatgccacgattgtggccaccctgaccgctgcctgtgcccagcactgcccggaagccatgatctgcgtcattgccaatccggttaattccaccatccccatcacagcagaagttttcaagaagcatggagtgtacaaccccaacaaaatcttcggcgtgacgaccctggacatcgtcagagccaacacctttgttgcagagctgaagggtttggatccagctcgagtcaacgtccctgtcattggtggccatgctgggaagaccatcatccccctgatctctcagtgcacccccaaggtggactttccccaggaccagctgacagcactcactgggcggatccaggaggccggcacggaggtggtcaaggctaaagccggagcaggctctgccaccctctccatggcgtatgccggcgcccgctttgtcttctcccttgtggatgcaatgaatggaaaggaaggtgttgtggaatgttccttcgttaagtcacaggaaacggaatgtacctacttctccacaccgctgctgcttgggaaaaagggcatcgagaagaacctgggcatcggcaaagtctcctcttttgaggagaagatgatctcggatgccatccccgagctgaaggcctccatcaagaagggggaagatttcgtgaagaccctgaagtga

SEQ ID NO: 6 - protein, 72 aa, Homo sapiens
KAKAGAGSATLSMAYAGARFVFSLVDAMNGKEGVVECSFVKSQETECTYFSTPLLLGKKGIEKNLGIGKVSS

SEQ ID NO: 7 - protein, 100 aa, Homo sapiens
KAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 8 - protein, 361 aa, Homo sapiens
MAAPRAGRGAGWSLRAWRALGGIRWGRRPRLTPDLRALLTSGTSDPRARVTYGTPSLWARLSVGVTEPRACLTSGTPGPRAQLTAVTPDTRTREASENSGTRSRAWLAVALGAGGAVLLLLWGGGRGPPAVLAAVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKFGNSGGPLVNLDGEVIGVNTMKVTAGISFAIPSDRLREFLHRGEKKNSSSGISGSQRRYIGVMMLTLSPRAGLRPGDVILAIGEQMVQNAEDVYEAVRTQSQLAVQIRRGRETLTLYVTPEVTE

SEQ ID NO: 9 - DNA, 1086 bp, Homo sapiens
atggctgcgccgagggcggggcggggtgcaggctggagccttcgggcatggcgggctttggggggcattcgctgggggaggagaccccgtttgacccctgacctccgggccctgctgacgtcaggaacttctgacccccgggcccgagtgacttatgggacccccagtctctgggcccggttgtctgttggggtcactgaaccccgagcatgcctgacgtctgggaccccgggtccccgggcacaactgactgcggtgaccccagataccaggacccgggaggcctcagagaactctggaacccgttcgcgcgcgtggctggcggtggcgctgggcgctgggggggcagtgctgttgttgttgtggggcgggggtcggggtcctccggccgtcctcgccgccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaagtttggaaactctggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaatga

SEQ ID NO: 10 - protein, 613 aa, Homo sapiens
MFRCGGLAAGALKQKLVPLVRTVCVRSPRQRNRLPGNLFQRWHVPLELQMTRQMASSGASGGKIDNSVLVLIVGLSTVGAGAYAYKTMKEDEKRYNERISGLGLTPEQKQKKAALSASEGEEVPQDKAPSHVPFLLIGGGTAAFAAARSIRARDPGARVLIVSEDPELPYMRPPLSKELWFSDDPNVTKTLRFKQWNGKERSIYFQPPSFYVSAQDLPHIENGGVAVLTGKKVVQLDVRDNMVKLNDGSQITYEKCLIATGGTPRSLSAIDRAGAEVKSRTTLFRKIGDFRSLEKISREVKSITIIGGGFLGSELACALGRKARALGTEVIQLFPEKGNMGKILPEYLSNWTMEKVRREGVKVMPNAIVQSVGVSSGKLLIKLKDGRKVETDHIVAAVGLEPNVELAKTGGLEIDSDFGGFRVNAELQARSNIWVAGDAACFYDIKLGRRRVEHHDHAVVSGRLAGENMTGAAKPYWHQSMFWSDLGPDVGYEAIGLVDSSLPTVGVFAKATAQDNPKSATEQSGTGIRSESETESEASEITIPPSTPAVPQAPVQGEDYGKGVIFYLRDKVVVGIVLWNIFNRMPIARKIIKDGEQHEDLNEVAKLFNIHED

SEQ ID NO: 11 - DNA, 1842 bp, Homo sapiens
atgttccggtgtggaggcctggcggcgggtgctttgaagcagaagctggtgcccttggtgcggaccgtgtgcgtccgaagcccgaggcagaggaaccggctcccaggcaacttgttccagcgatggcatgttcctctagaactccagatgacaagacaaatggctagctctggtgcatcagggggcaaaatcgataattctgtgttagtccttattgtgggcttatcaacagtaggagctggtgcctatgcctacaagactatgaaagaggacgaaaaaagatacaatgaaagaatttcagggttagggctgacaccagaacagaaacagaaaaaggccgcgttatctgcttcagaaggagaggaagttcctcaagacaaggcgccaagtcatgttcctttcctgctaattggtggaggcacagctgcttttgctgcagccagatccatccgggctcgggatcctggggccagggtactgattgtatctgaagatcctgagctgccgtacatgcgacctcctctttcaaaagaactgtggttttcagatgacccaaatgtcacaaagacactgcgattcaaacagtggaatggaaaagagagaagcatatatttccagccaccttctttctatgtctctgctcaggacctgcctcatattgagaatggtggtgtggctgtcctcactgggaagaaggtagtacagctggatgtgagagacaacatggtgaaacttaatgatggctctcaaataacctatgaaaagtgcttgattgcaacaggaggtactccaagaagtctgtctgccattgatagggctggagcagaggtgaagagtagaacaacgcttttcagaaagattggagactttagaagcttggagaagatttcacgggaagtcaaatcaattacgattatcggtgggggcttccttggtagcgaactggcctgtgctcttggcagaaaggctcgagccttgggcacagaagtgattcaactcttccccgagaaaggaaatatgggaaagatcctccccgaatacctcagcaactggaccatggaaaaagtcagacgagagggggttaaggtgatgcccaatgctattgtgcaatccgttggagtcagcagtggcaagttacttatcaagctgaaagacggcaggaaggtagaaactgaccacatagtggcagctgtgggcctggagcccaatgttgagttggccaagactggtggcctggaaatagactcagattttggtggcttccgggtaaatgcagagctacaagcacgctctaacatctgggtggcaggagatgctgcatgcttctacgatataaagttgggaaggaggcgggtagagcaccatgatcacgctgttgtgagtggaagattggctggagaaaatatgactggagctgctaagccgtactggcatcagtcaatgttctggagtgatttgggccccgatgttggctatgaagctattggtcttgtggacagtagtttgcccacagttggtgtttttgcaaaagcaactgcacaagacaaccccaaatctgccacagagcagtcaggaactggtatccgatcagagagtgagacagagtccgaggcctcagaaattactattcctcccagcaccccggcagttccacaggctcccgtccagggggaggactacggcaaaggtgtcatcttctacctcagggacaaagtggtcgtggggattgtgctatggaacatctttaaccgaatgccaatagcaaggaagatcattaaggacggtgagcagcatgaagatctcaatgaagtagccaaactattcaacattcatgaagactga

SEQ ID NO: 12 - protein, 186 aa, Homo sapiens
MKSDFYFQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 13 - DNA, 561 bp, Homo sapiens
atgaaatctgacttctacttccagaaatcagagcctcattcccttagtagtgaagcattgatgaggagagcagtgtctttggtaacagatagcacctctacctttctctctcagaccacatatgcgttgattgaagctattactgaatatactaaggctgtttataccttaacttctctttaccgacaatatacaagtttacttgggaaaatgaattcagaggaggaagatgaagtgtggcaggtgatcataggagccagagctgagatgacttcaaaacaccaagagtacttgaagctggaaaccacttggatgactgcagttggtctttcagagatggcagcagaagctgcatatcaaactggcgcagatcaggcctctataaccgccaggaatcacattcagctggtgaaactgcaggtggaagaggtgcaccagctctcccggaaagcagaaaccaagctggcagaagcacagatagaagagctccgtcagaaaacacaggaggaaggggaggagcgggctgagtcggagcaggaggcctacctgcgtgaggattga

SEQ ID NO: 14 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 15 - DNA, 2349 bp, Homo sapiens
atggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctga

SEQ ID NO: 16 - protein, 239 aa, Homo sapiens
MAHAGRTGYDNREIVMKYIHYKLSQRGYEWDAGDVGAAPPGAAPAPGIFSSQPGHTPHTAASRDPVARTSPLQTPAAPGAAAGPALSPVPPVVHLTLRQAGDDFSRRYRRDFAEMSRQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 17 - DNA, 720 bp, Homo sapiens
atggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 18 - protein, 164 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKAGVKWRDLGSLQPLPPGFKRFTCLSIPRSWDYRPCAPRCRN

SEQ ID NO: 19 - DNA, 495 bp, Homo sapiens
atggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggctggcgtgaaatggcgtgatctgggctcactgcaacctctgcctcctgggttcaagcgattcacctgcctcagcatcccaaggagctgggattacaggccctgtgcaccaaggtgccggaactga

SEQ ID NO: 20 - protein, 168 aa, Homo sapiens
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 21 - DNA, 507 bp, Homo sapiens
atgttccagatcccagagtttgagccgagtgagcaggaagactccagctctgcagagaggggcctgggccccagccccgcaggggacgggccctcaggctccggcaagcatcatcgccaggccccaggcctcctgtgggacgccagtcaccagcaggagcagccaaccagcagcagccatcatggaggcgctggggctgtggagatccggagtcgccacagctcctaccccgcggggacggaggacgacgaagggatgggggaggagcccagcccctttcggggccgctcgcgctcggcgccccccaacctctgggcagcacagcgctatggccgcgagctccggaggatgagtgacgagtttgtggactcctttaagaagggacttcctcgcccgaagagcgcgggcacagcaacgcagatgcggcaaagctccagctggacgcgagtcttccagtcctggtgggatcggaacttgggcaggggaagctccgccccctcccagtga

SEQ ID NO: 22 - protein, 241 aa, Homo sapiens
MCSGAGVMMARWAARGRAGWRSTVRILSPLGHCEPGVSRSCRAAQAMDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 23 - DNA, 726 bp, Homo sapiens
atgtgcagcggtgctggggtcatgatggctcggtgggcagcgaggggccgggccggctggaggagcacagtgcggattctgtcgccactgggacactgtgaaccaggagtgagtcggagctgccgcgctgcccaggccatggactgtgaggtcaacaacggttccagcctcagggatgagtgcatcacaaacctactggtgtttggcttcctccaaagctgttctgacaacagcttccgcagagagctggacgcactgggccacgagctgccagtgctggctccccagtgggagggctacgatgagctgcagactgatggcaaccgcagcagccactcccgcttgggaagaatagaggcagattctgaaagtcaagaagacatcatccggaatattgccaggcacctcgcccaggtcggggacagcatggaccgtagcatccctccgggcctggtgaacggcctggccctgcagctcaggaacaccagccggtcggaggaggaccggaacagggacctggccactgccctggagcagctgctgcaggcctaccctagagacatggagaaggagaagaccatgctggtgctggccctgctgctggccaagaaggtggccagtcacacgccgtccttgctccgtgatgtctttcacacaacagtgaattttattaaccagaacctacgcacctacgtgaggagcttagccagaaatgggatggactga

SEQ ID NO: 24 - protein, 297 aa, Homo sapiens
MRALRAGLTLASGAGLGAVVEGWRRRREDARAALGLLGRLPVLPVAAAAELPPVPGGPRGPGELAKYGLPGLAQLKSRESYVLCYDPRTRGALWVVEQLRPERLRGDGDRRECDFREDDSVHAYHRATNADYRGSGFDRGHLAAAANHRWSQKAMDDTFYLSKVAPQVPHLNQNAWNNLEKYSRSLTRSYQNVYVCTGPLFLPRTEADGKSYVKYQVIGKNHVAVPTHFFKVLILEAAGGQIELRTYVMPNAPVDEAIPLERFLVPIESIERASGLLFVPNILARAGSLKAITAGSK

SEQ ID NO: 25 - DNA, 894 bp, Homo sapiens
atgcgggcgctgcgggccggcctgaccctggcgtcgggcgcggggctgggtgcggtcgtcgagggctggcggcggcggcgggaggacgcgcgggcggcgctgggactgctgggccggctgcccgtgctgcccgtggcggcggcagccgagttgccccctgtgcccgggggaccccgcggcccgggcgagttggccaagtacgggctgccggggctggcgcagctcaagagccgcgagtcgtacgtgctgtgctacgacccgcgcacccgcggcgcgctctgggtggtggagcagctgcgacccgagcgtctccgcggcgacggcgaccggcgcgagtgcgacttccgcgaggacgactcggtgcacgcgtaccaccgtgccaccaacgccgactaccgcggcagtggcttcgaccgcggtcacctggccgccgccgccaaccaccgctggagccagaaggccatggacgacacgttctacctgagcaaagtcgcgccccaggtgccccacctcaaccagaatgcctggaacaacctggagaaatatagccgcagcttgacccgcagctaccaaaacgtctatgtctgcacagggccactcttcctgcccaggacagaggctgatgggaaatcctacgtaaagtaccaggtcatcggcaagaaccacgtggcagtgcccacacacttcttcaaggtgctgatcctggaggcagcaggtgggcaaattgagctccgcacctacgtgatgcccaacgcacctgtggatgaggccatcccactggagcgcttcctggtgcccatcgagagcattgagcgggcttcggggctgctctttgtgccaaacatcctggcgcgggcaggcagcctcaaggccatcacggcgggcagtaagtga

SEQ ID NO: 26 - protein, 338 aa, Homo sapiens
MLQKPKSVKLRALRSPRKFGVAGRSCQEVLRKGCLRFQLPERGSRLCLYEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 27 - DNA, 1017 bp, Homo sapiens
atgctccagaagcccaagagcgtgaagctgcgggccctgcgcagcccgaggaagttcggcgtggctggccggagctgccaggaggtgctgcgcaagggctgtctccgcttccagctccctgagcgcggttcccggctgtgcctgtacgaggatggcacggagctgacggaagattacttccccagtgttcccgacaacgccgagctggtgctgctcaccttgggccaggcctggcagggctatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagacccgggctgaggacccgccgtggtttgaaggcttggagtcccgatttcagagcaagtctggctatctgagatacagctgtgagagccggatccggagttacctgagggaggtgagctcctacccctccacggtgggtgcggaggctcaggaggaattcctgcgggtcctcggctccatgtgccagaggctccggtccatgcagtacaatggcagctacttcgacagaggagccaagggcggcagccgcctctgcacaccggaaggctggttctcctgccagggtccctttgacatggacagctgcttatcaagacactccatcaacccctacagtaacagggagagcaggatcctcttcagcacctggaacctggatcacataatagaaaagaaacgcaccatcattcctacactggtggaagcaattaaggaacaagatggaagagaagtggactgggagtatttttatggcctgctttttacctcagagaacctaaaactagtgcacattgtctgccataagaaaaccacccacaagctcaactgtgacccaagcagaatctacaaaccccagacaaggttgaagcggaagcagcctgtgcggaaacgccagtga

SEQ ID NO: 28 - protein, 331 aa, Homo sapiens
MEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPT

SEQ ID NO: 29 - DNA, 996 bp, Homo sapiens
atggaggtgaccggggacgccggggtaccagaatctggcgagatccggactctaaagccgtgtctgctgcgccgcaactacagccgcgaacagcacggcgtggccgcctcctgcctcgaagacctgaggagcaaggcctgtgacattctggccattgataagtccctgacaccagtcaccctggtcctggcagaggatggcaccatagtggatgatgacgattactttctgtgtctaccttccaatactaagtttgtggcattggctagtaatgagaaatgggcatacaacaattcagatggaggtacagcttggatttcccaagagtcctttgatgtagatgaaacagacagcggggcagggttgaagtggaagaatgtggccaggcagctgaaagaagatctgtccagcatcatcctcctatcagaggaggacctccagatgcttgttgacgctccctgctcagacctggctcaggaactacgtcagagttgtgccaccgtccagcggctgcagcacacactccaacaggtgcttgaccaaagagaggaagtgcgtcagtccaagcagctcctgcagctgtacctccaggctttggagaaagagggcagcctcttgtcaaagcaggaagagtccaaagctgcctttggtgaggaggtggatgcagtagacacgggtatcagcagagagacctcctcggacgttgcgctggcgagccacatccttactgcactgagggagaagcaggctccagagctgagcttatctagtcaggatttggagttggttaccaaggaagaccccaaagcactggctgttgccttgaactgggacataaagaagacggagactgttcaggaggcctgtgagcgggagctcgccctgcgcctgcagcagacgcagagcttgcattctctccggagcatctcagcaagcaaggcctcaccacctggtgacctgcagaatcctaagcgagccagacaggatcccacatag

SEQ ID NO: 30 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 31 - DNA, 3624 bp, Homo sapiens
atgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaataagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggtgcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtga

SEQ ID NO: 32 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 33 - DNA, 576 bp, Homo sapiens
atgaactttctgctgtcttgggtgcattggagccttgccttgctgctctacctccaccatgccaagtggtcccaggctgcacccatggcagaaggaggggggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 34 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 35 - DNA, 528 bp, Homo sapiens
atggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatag

SEQ ID NO: 36 - protein, 378 aa, Drosophila melanogaster
MTMSTNNCESMTSYFTNSYMGADMHHGHYPGNGVTDLDAQQMHHYSQNANHQGNMPYPRFPPYDRMPYYNGQGMDQQQQHQVYSRPDSPSSQVGGVMPQAQTNGQLGVPQQQQQQQQQPSQNQQQQQAQQAPQQLQQQLPQVTQQVTHPQQQQQQPVVYASCKLQAAVGGLGMVPEGGSPPLVDQMSGHHMNAQMTLPHHMGHPQAQLGYTDVGVPDVTEVHQNHHNMGMYQQQSGVPPVGAPPQGMMHQGQGPPQMHQGHPGQHTPPSQNPNSQSSGMPSPLYPWMRSQFGKCQERKRGRQTYTRYQTLELEKEFHFNRYLTRRRRIEIAHALCLTERQIKIWFQNRRMKWKKENKTKGEPGSGGEGDEITPPNSPQ

SEQ ID NO: 37 - DNA, 1137 bp, Drosophila melanogaster
atgacgatgagtacaaacaactgcgagagcatgacctcgtacttcaccaactcgtacatgggggcggacatgcatcatgggcactacccgggcaacggggtcaccgacctggacgcccagcagatgcaccactacagccagaacgcgaatcaccagggcaacatgccctacccgcgctttccaccctacgaccgcatgccctactacaacggccaggggatggaccagcagcagcagcaccaggtctactcccgcccggacagcccctccagccaggtgggcggggtcatgccccaggcgcagaccaacggtcagttgggtgttccccagcagcaacagcagcagcagcaacagccctcgcagaaccagcagcaacagcaggcgcagcaggccccacagcaactgcagcagcagctgccgcaggtgacgcaacaggtgacacatccgcagcagcaacaacagcagcccgtcgtctacgccagctgcaagttgcaagcggccgttggtggactgggtatggttcccgagggcggatcgcctccgctggtggatcaaatgtccggtcaccacatgaacgcccagatgacgctgccccatcacatgggacatccgcaggcgcagttgggctatacggacgttggagttcccgacgtgacagaggtccatcagaaccatcacaacatgggcatgtaccagcagcagtcgggagttccgccggtgggtgccccacctcagggcatgatgcaccagggccagggtcctccacagatgcaccagggacatcctggccaacacacgcctccttcccaaaacccgaactcgcagtcctcggggatgccgtctccactgtatccctggatgcgaagtcagtttggtaagtgtcaagaacgcaaacgcggaaggcagacatacacccggtaccagactctagagctagagaaggagtttcacttcaatcgctacttgacccgtcggcgaaggatcgagatcgcccacgccctgtgcctcacggagcgccagataaagatttggttccagaatcggcgcatgaagtggaagaaggagaacaagacgaagggcgagccgggatccggaggcgaaggcgacgagataacaccacccaacagtccgcagtag

SEQ ID NO: 38 - protein, 163 aa, Homo sapiens
MSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 39 - DNA, 492 bp, Homo sapiens
atgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtag

SEQ ID NO: 40 - protein, 282 aa, Homo sapiens [sequence omitted]

SEQ ID NO: 41 - DNA, 849 bp, Homo sapiens
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 42 - protein, 360 aa, Homo sapiens
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 43 - DNA, 1083 bp, Homo sapiens
atgatcccgctgctgctggcagcgctgctgtgcgtccccgccggggccctgacctgctacggggactccgggcagcctgtagactggttcgtggtctacaagctgccagctcttagagggtccggggaggcggcgcagagagggctgcagtacaagtatctggacgagagctccggaggctggcgggacggcagggcactcatcaacagcccggagggggccgtgggccgaagcctgcagccgctgtaccggagcaacaccagccagctcgccttcctgctctacaatgaccaaccgcctcaacccagcaaggctcaggactcttccatgcgtgggcacacgaagggtgtcctgctccttgaccacgatgggggcttctggctggtccacagtgtacctaacttccctccaccggcctcctctgctgcatacagctggcctcatagcgcctgtacctacgggcagaccctgctctgtgtgtcttttcccttcgctcagttctcgaagatgggcaagcagctgacctacacctacccctgggtctataactaccagctggaagggatctttgcccaggaattccccgacttggagaatgtggtcaagggccaccacgttagccaagaaccctggaacagcagcatcacactcacatcccaggccggggctgttttccagagctttgccaagttcagcaaatttggagatgacctgtactccggctggttggcagcagcccttggtaccaacctgcaggtccagttctggcacaaaactgtaggcatcctgccctctaactgctcggatatctggcaggttctgaatgtgaaccagatagctttccctggaccagccggcccaagcttcaacagcacagaggaccactccaaatggtgcgtgtccccaaaagggccctggacctgcgtgggtgacatgaatcggaaccagggagaggagcaacggggtgggggcacactgtgtgcccagctgccagccctctggaaagccttccagccgctggtgaagaactaccagccctgtaatggcatggccaggaagcccagcagagcttataagatctaa

SEQ ID NO: 44 - protein, 335 aa, Homo sapiens
MDNARMNSFLEYPILSSGDSGTCSARAYPSDHRITTFQSCAVSANSCGGDDRFLVGRGVQIGSPHHHHHHHHHHPQPATYQTSGNLGVSYSHSSCGPSYGSQNFSAPYSPYALNQEADVSGGYPQCAPAVYSGNLSSPMVQHHHHHQGYAGGAVGSPQYIHHSYGQEHQSLALATYNNSLSPLHASHQEACRSPASETSSPAQTFDWMKVKRNPPKTGKVGEYGYLGQPNAVRTNFTTKQLTELEKEFHFNKYLTRARRVEIAASLQLNETQVKIWFQNRRMKQKKREKEGLLPISPATPPGNDEKAEESSEKSSSSPCVPSPGSSTSDTLTTSH

SEQ ID NO: 45 - DNA, 1008 bp, Homo sapiens
atggacaatgcaagaatgaactccttcctggaataccccatacttagcagtggcgactcggggacctgctcagcccgagcctacccctcggaccataggattacaactttccagtcgtgcgcggtcagcgccaacagttgcggcggcgacgaccgcttcctagtgggcaggggggtgcagatcggttcgccccaccaccaccaccaccaccaccatcaccacccccagccggctacctaccagacttccgggaacctgggggtgtcctactcccactcaagttgtggtccaagctatggctcacagaacttcagtgcgccttacagcccctacgcgttaaatcaggaagcagacgtaagtggtgggtacccccagtgcgctcccgctgtttactctggaaatctctcatctcccatggtccagcatcaccaccaccaccagggttatgctgggggcgcggtgggctcgcctcaatacattcaccactcatatggacaggagcaccagagcctggccctggctacgtataataactccttgtcccctctccacgccagccaccaagaagcctgtcgctcccccgcatcggagacatcttctccagcgcagacttttgactggatgaaagtcaaaagaaaccctcccaaaacagggaaagttggagagtacggctacctgggtcaacccaacgcggtgcgcaccaacttcactaccaagcagctcacggaactggagaaggagttccacttcaacaagtacctgacgcgcgcccgcagggtggagatcgctgcatccctgcagctcaacgagacccaagtgaagatctggttccagaaccgccgaatgaagcaaaagaaacgtgagaaggagggtctcttgcccatctctccggccaccccgccaggaaacgacgagaaggccgaggaatcctcagagaagtccagctcttcgccctgcgttccttccccggggtcttctacctcagacactctgactacctcccactga

SEQ ID NO: 46 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 47 - DNA, 543 bp, Homo sapiens
atgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtga

SEQ ID NO: 48 - protein, 59 aa, Homo sapiens
MAEGEITTFTALTEKFNLPPGNYKKPKLLYCSNGGHFLRILPDGTVDGTRDRSDQHNTK

SEQ ID NO: 49 - DNA, 180 bp, Homo sapiens
atggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacaacaccaaatga

SEQ ID NO: 50 - protein, 102 aa, Human immunodeficiency virus
MEPVDPNLEPWNHPGSQPQTPCNKCYCKHCSYHCLVCFQTKGLGISYGRKKRRQRRSTPPSSESHQNPLSKQPLPQTRGDQTGSEEQKKKVESKTETDPYDW

SEQ ID NO: 51 - DNA, 30 bp, Artificial Sequence
  Synthetic
gaattcgcagatctgagccacatcgagacc

SEQ ID NO: 52 - DNA, 37 bp, Artificial Sequence
  Synthetic
gtcgactcagaccacctccgtgccggcctcctggatc

SEQ ID NO: 53 - DNA, 32 bp, Artificial Sequence
  Synthetic
gaattcaaggctaaagccggagcaggctctgc

SEQ ID NO: 54 - DNA, 36 bp, Artificial Sequence
  Synthetic
gtcgactcacttcagggtcttcacgaaatcttcccc

SEQ ID NO: 55 - DNA, 36 bp, Artificial Sequence
  Synthetic
ggccgaattcaaggctaaagccggagcaggctctgc

SEQ ID NO: 56 - DNA, 88 bp, Artificial Sequence
  Synthetic
aattgtcgacttattttttccatttcatgcggcggttctgaaaccaaattttaatctggcgcttcagggtcttcacgaaatcttcccc

SEQ ID NO: 57 - protein, 16 aa, Artificial Sequence
  Synthetic
RQIKIWFQNRRMKWKK

SEQ ID NO: 58 - protein, 36 aa, Human immunodeficiency virus type 1
CFITKALGISYGRKKRRQRRRPPQGSQTHQVSLSKQ

SEQ ID NO: 59 - protein, 14 aa, Artificial Sequence
  Synthetic
GRKKRRQRRRPPQC

SEQ ID NO: 60 - protein, 17 aa, Human immunodeficiency virus type 1
TRQARRNRRRRWRERQR

SEQ ID NO: 61 - protein, 21 aa, Artificial Sequence
  Synthetic
KETWWETWWTEWSQPKKKRKV

SEQ ID NO: 62 - protein, 7 aa, Artificial Sequence
  Synthetic
PKKKRKV

SEQ ID NO: 63 - protein, 9 aa, Artificial Sequence
  Synthetic
PAAKRVKLD

SEQ ID NO: 64 - protein, 12 aa, Human immunodeficiency virus type 1
GRKKRRQRRRAP

SEQ ID NO: 65 - protein, 8 aa, Artificial Sequence
  Synthetic
PLLKKIKQ

SEQ ID NO: 66 - protein, 8 aa, Artificial Sequence
  Synthetic
PPQKKIKS

SEQ ID NO: 67 - protein, 7 aa, Artificial Sequence
  Synthetic
PQPKKKP

SEQ ID NO: 68 - protein, 9 aa, Artificial Sequence
  Synthetic
SKRVAKRKL

SEQ ID NO: 69 - protein, 5 aa, Artificial Sequence
  Synthetic
GRRRR

SEQ ID NO: 70 - protein, 155 aa, Homo sapiens [sequence omitted]

SEQ ID NO: 71 - DNA, 6757 bp, Homo sapiens
cggccccagaaaacccgagcgagtagggggcggcgcgcaggagggaggagaactgggggcgcgggaggctggtgggtgtcgggggtggagatgtagaagatgtgacgccgcggcccggcgggtgccagattagcggacggctgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcattcatctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgcttgaaaataaattatggggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggtttcctggtgtttcctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttatactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaattcatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgacttcctgactattaaaaataaatagtagaaacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctccttttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtg

SEQ ID NO: 72 - protein, 513 aa, Homo sapiens
MFADRWLFSTNHKDIGTLYLLFGAWAGVLGTALSLLIRAELGQPGNLLGNDHIYNVIVTAHAFVMIFFMVMPIMIGGFGNWLVPLMIGAPDMAFPRMNNMSFWLLPPSLLLLLASAMVEAGAGTGWTVYPPLAGNYSHPGASVDLTIFSLHLAGVSSILGAINFITTIINMKPPAMTQYQTPLFVWSVLITAVLLLLSLPVLAAGITMLLTDRNLNTTFFDPAGGGDPILYQHLFWFFGHPEVYILILPGFGMISHIVTYYSGKKEPFGYMGMVWAMMSIGFLGFIVWAHHMFTVGMDVDTRAYFTSATMIIAIPTGVKVFSWLATLHGSNMKWSAAVLWALGFIFLFTVGGLTGIVLANSSLDIVLHDTYYVVAHFHYVLSMGAVFAIMGGFIHWFPLFSGYTLDQTYAKIHFTIMFIGVNLTFFPQHFLGLSGMPRRYSDYPDAYTTWNILSSVGSFISLTAVMLMIFMIWEAFASKRKVLMVEEPSMNLEWLYGCPPPYHTFEEPVYMKS

SEQ ID NO: 73 - DNA, 1542 bp, Homo sapiens
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctaga

SEQ ID NO: 74 - protein, 219 aa, Homo sapiens
MSSHLVEPPPPLHNNNNNCEENEQSLPPPAGLNSSWVELPMNSSNGNDNGNGKNGGLEHVPSSSSIHNGDMEKILLDAQHESGQSSSRGSSHCDSPSPQEDGQIMFDVEMHTSRDHSSQSEEEVVEGEKEVEALKKSADWVSDWSSRPENIPPKEFHFRHPKRSVSLSMRKSGAMKKGGIFSAEFLKVFIPSLFLSHVLALGLGIYIGKRLSTPSASTY

SEQ ID NO: 75 - DNA, 660 bp, Homo sapiens
atgtcgtcccacctagtcgagccgccgccgcccctgcacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcttttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttcagacaccctaaacgttctgtgtctttaagcatgaggaaaagtggagccatgaagaaagggggtattttctccgcagaatttctgaaggtgttcattccatctctcttcctttctcatgttttggctttggggctaggcatctatattggaaagcgactgagcacaccctctgccagcacctactga

SEQ ID NO: 76 - protein, 194 aa, Homo sapiens
MSQNGAPGMQEESLQGSWVELHFSNNGNGGSVPASVSIYNGDMEKILLDAQHESGRSSSKSSHCDSPPRSQTPQDTNRASETDTHSIGEKNSSQSEEDDIERRKEVESILKKNSDWIWDWSSRPENIPPKEFLFKHPKRTATLSMRNTSVMKKGGIFSAEFLKVFLPSLLLSHLLAIGLGIYIGRRLTTSTSTF

SEQ ID NO: 77 - DNA, 585 bp, Homo sapiens
atgtcgcagaacggagcgcccgggatgcaggaggagagcctgcagggctcctgggtagaactgcacttcagcaataatgggaacgggggcagcgttccagcctcggtttctatttataatggagacatggaaaaaatactgctggacgcacagcatgagtctggacggagtagctccaagagctctcactgtgacagcccacctcgctcgcagacaccacaagataccaacagggcttctgaaacagatacccatagcattggagagaaaaacagctcacagtctgaggaagatgatattgaaagaaggaaagaagttgaaagcatcttgaagaaaaactcagattggatatgggattggtcaagtcggccggaaaatattccccccaaggagttcctctttaaacacccgaagcgcacggccaccctcagcatgaggaacacgagcgtcatgaagaaagggggcatattctctgcagaatttctgaaagttttccttccatctctgctgctctctcatttgctggccatcggattggggatctatattggaaggcgtctgacaacctccaccagcaccttttga

SEQ ID NO: 78 - protein, 219 aa, Homo sapiens
MEYLSALNPSDLLRSVSNISSEFGRRVWTSAPPPQRPFRVCDHKRTIRKGLTAATRQELLAKALETLLLNGVLTLVLEEDGTAVDSEDFFQLLEDDTCLMVLQSGQSWSPTRSGVLSYGLGRERPKHSKDIARFTFDVYKQNPRDLFGSLNVKATFYGLYSMSCDFQGLGPKKVLRELLRWTSTLLQGLGHMLLGISSTLRHAVEGAEQWQQKGRLHSY

SEQ ID NO: 79 - DNA, 660 bp, Homo sapiens
atggagtacctctcagctctgaaccccagtgacttactcaggtcagtatctaatataagctcggagtttggacggagggtctggacctcagctccaccaccccagcgacctttccgtgtctgtgatcacaagcggaccatccggaaaggcctgacagctgccacccgccaggagctgctagccaaagcattggagaccctactgctgaatggagtgctaaccctggtgctagaggaggatggaactgcagtggacagtgaggacttcttccagctgctggaggatgacacgtgcctgatggtgttgcagtctggtcagagctggagccctacaaggagtggagtgctgtcatatggcctgggacgggagaggcccaagcacagcaaggacatcgcccgattcacctttgacgtgtacaagcaaaaccctcgagacctctttggcagcctgaatgtcaaagccacattctacgggctctactctatgagttgtgactttcaaggacttggcccaaagaaagtactcagggagctccttcgttggacctccacactgctgcaaggcctgggccatatgttgctgggaatttcctccacccttcgtcatgcagtggagggggctgagcagtggcagcagaagggccgcctccattcctactaa

SEQ ID NO: 80 - protein, 242 aa, Artificial Sequence
  Synthetic
QVHLIQAGPGLVQPSQSLSITCTVSGLSLINYGVHWVRQSPGKGLEWLGVIWSGGSTDYNAAFISRLSISKDNSKSQVFFKMNSLQGNDTAIYYCARNSELGAMDYWAQGISVTVSSGGGGSGGGGSGGGGSDIVLTQSPASLAVSLGQRATISCRASKSVSTSGYSYMHWNQQKPGQPPRLLIYLVSNLESGVPARFSGSGSGTDFTLNIHPVEEEDAATYYCQHIREAYTFGGGTKLEIK

SEQ ID NO: 81 - DNA, 756 bp, Artificial Sequence
  Synthetic
gcaatactccatgggccaggtgcatctgattcaggcgggcccgggcctggtgcagccgagccagagcctgagcattacctgcaccgtgagcggcctgagcctgattaactatggcgtgcattgggtgcgtcagagcccgggcaaaggcctggaatggctgggcgtgatttggagcggcggcagcaccgattataacgcggcgtttattagccgtctgagcattagcaaagataacagcaaaagccaggtgttttttaaaatgaacagcctgcagggcaacgataccgcgatttattattgcgcgcgtaacagcgaactgggcgcgatggattattgggcgcagggcattagcgtgaccgtgagcagcggcggcggcggcagcggcggcggcggcagcggcggcggcggcagcgatattgtgctgacccagagcccggcgagcctggcggtgagcctgggccagcgtgcgaccattagctgccgtgcgagcaaaagcgtgagcaccagcggctatagctatatgcattggaaccagcagaaaccgggccagccgccgcgtctgctgatttatctggtgagcaacctggaaagcggcgtgccggcgcgttttagcggcagcggcagcggcaccgattttaccctgaacattcatccggtggaagaagaagatgcggcgacctattattgccagcatattcgtgaagcgtatacctttggcggcggcaccaaactggaaattaaactcgaggcatagcc